(19) United States
(12) Patent Application Publication
(10) Pub. No.: US 2026/0088149 A1
(43) Pub. Date: Mar. 26, 2026

(54) FLOW METER AND RELATED METHOD

(71) Applicants: Bob D. Peret, Bedford, NH (US); Derek G. Kane, Manchester, NH (US); Dean Kamen, Bedford, NH (US); Colin H. Murphy, Cambridge, MA (US); John M. Kerwin, Manchester, NH (US); Karla Beagle, Bedford, NH (US); Dirk A. Van Der Merwe, Canterbury, NH (US); Gregory J. Buitkus, North Andover, MA (US); Daniel S. Karol, Manchester, NH (US); Drew R. Blais, Hampstead, NH (US); Samantha Pinella, Manchester, NH (US); Bryan I. Stoneham, Stoneham, MA (US); Adnan Suljevic, Manchester, NH (US); Naveen Mitikiri, Manchester, NH (US)

(72) Inventors: Bob D. Peret, Bedford, NH (US); Derek G. Kane, Manchester, NH (US); Dean Kamen, Bedford, NH (US); Colin H. Murphy, Cambridge, MA (US); John M. Kerwin, Manchester, NH (US); Karla Beagle, Bedford, NH (US); Dirk A. Van Der Merwe, Canterbury, NH (US); Gregory J. Buitkus, North Andover, MA (US); Daniel S. Karol, Manchester, NH (US); Drew R. Blais, Hampstead, NH (US); Samantha Pinella, Manchester, NH (US); Bryan I. Stoneham, Stoneham, MA (US); Adnan Suljevic, Manchester, NH (US); Naveen Mitikiri, Manchester, NH (US)

(73) Assignee: DEKA Products Limited Partnership, Manchester, NH (US)

(21) Appl. No.: 19/372,932

(22) Filed: Oct. 29, 2025

Related U.S. Application Data
(63) Continuation of application No. 18/890,929, filed on Sep. 20, 2024.
(63) Continuation of application No. 17/134,854, filed on Dec. 28, 2020, now Pat. No. 12,100,507.
(63) Continuation-in-part of application No. 18/241,573, filed on Sep. 1, 2023, now Pat. No. 12,502,476.
(60) Provisional application No. 63/713,162, filed on Oct. 29, 2024.
(60) Provisional application No. 63/753,746, filed on Feb. 4, 2025.

(51) Int. Cl. G16H 20/17; A61B 17/00; A61M 5/142; A61M 5/168; A61M 25/00; F04B 43/08; F04B 43/09; F04B 43/12; F04B 49/00; F04B 49/06; G01F 1/00; G01F 1/66; G06Q 50/22; G16H 30/00; G16H 40/63; G16H 40/67; G16H 50/00; G16Z 99/00

(52) U.S. Cl. CPC G16H 20/17; A61B 17/00234; A61M 5/14228; A61M 5/16831; A61M 5/16877; A61M 5/16886; A61M 25/00; F04B 43/08; F04B 43/082; F04B 43/12; F04B 43/1261; F04B 49/00; F04B 49/065; G01F 1/00; G01F 1/666; G06Q 50/22; G16H 30/00; G16H 40/63; G16H 40/67; G16H 50/00; G16Z 99/00; A61M 2005/16863; F04B 43/09; F04B 2205/09; G06T 2207/20104

(57) ABSTRACT

This disclosure relates to a gravity-driven infusion system that uses image-based monitoring to regulate flow. A contrasting, infrared-backlit wall and camera capture pendant drops and meniscus levels within a transparent drip chamber. The controller defines a baseline referenced to the spout or meniscus and fits sparse spline points on the drop perimeter to derive geometric functionals, such as neck width and centroid height. Temporal changes of these functionals map directly to instantaneous flow without explicit volume integration. An optional Young-Laplace model provides a physics-based boundary and confidence metric. A meniscus trend yields a low-frequency flow estimate. Confidence-weighted fusion controls a flow-control valve that compresses a multi-lumen insert. An independent safety occluder and watchdog ensure fail-safe shutdown. A medication library stores fluid-aware calibration. Multi-source embodiments orchestrate multiple controllers with virtual head-height equalization and verified handoffs. It performs pre-infusion checks, logs uncertainty, and supports tilt compensation too. Continuous stream detection triggers alarms.

### CROSS-REFERENCE TO RELATED APPLICATIONS

This application claims priority under 35 U.S.C. § 119(e) and Article 8 of the Paris Convention to U.S. Provisional Patent Application No. 63/713,162, filed Oct. 29, 2024; and U.S. Provisional Patent Application No. 63/753,746, filed Feb. 4, 2025, each of which is incorporated by reference herein in its entirety.

This application is a continuation-in-part of U.S. patent application Ser. No. 18/890,929 (pending), which is a continuation of U.S. patent application Ser. No. 17/134,854 (now U.S. Pat. No. 10,876,868); which is a continuation of U.S. patent application Ser. No. 16/136,753 (now U.S. Pat. No. 10,088,346); which is a continuation of U.S. patent application Ser. No. 14/812,149 (now U.S. Pat. No. 9,151,646); which is a continuation-in-part of U.S. patent application Ser. No. 13/723,244 (claiming the benefit of U.S. Provisional Patent Application Nos. 61/578,649, 61/578,658, 61/578,674, 61/651,322, and 61/679,117) and is a continuation-in-part of International Application No. PCT/US11/66588. U.S. patent application Ser. No. 14/812,149 is also a continuation-in-part of U.S. patent application Ser. Nos. 13/723,238, 13/723,235, 13/724,568, 13/725,790, 13/723,239, 13/723,242, 13/723,251, and 13/723,253.

This application is also a continuation-in-part of U.S. patent application Ser. No. 18/241,573 (a continuation of U.S. patent application Ser. No. 15/418,096, now U.S. Pat. No. 11,744,935), which claims the benefit of U.S. Provisional Patent Application Nos. 62/288,132 and 62/341,396, the entire contents of each of which are incorporated herein by reference.

The disclosures of the applications identified above, and any documents cited therein, are incorporated by reference in their entireties for all purposes to the extent permitted by law. In the event of a conflict between any incorporated material and the present disclosure, the present disclosure controls.

### BACKGROUND

### Technical Field

The present generally relates to systems, apparatuses, and methods for monitoring and regulating fluid flow, and more particularly to gravity-driven infusion systems that use optical sensing of drop formation within a drip chamber to determine a real-time flowrate.

### Description of Related Art

Gravity-fed infusion systems are widely used to deliver medical fluids without mechanical pumps. In a typical setup, a suspended fluid bag feeds a drip chamber that releases fluid as individual drops into a downstream tube. Flow rate is commonly inferred by counting drops over time and applying a nominal calibration factor such as 20 drops=1 milliliter. This approach is inexpensive but inherently approximate: drop size varies with fluid viscosity, surface tension, temperature, and manufacturing tolerances of the chamber, resulting in large dose errors at low flow rates.

Manual roller or slide clamps are normally used to regulate flow in these systems. Their non-linear compression characteristics make precise adjustment difficult, and changes in bag head height or patient pressure can further alter flow. Electronic drop counters have been introduced to automate measurement, but they generally rely on optical interruption sensors that detect the passage of detached drops through a light beam. Such sensors provide only discrete measurements and cannot estimate flow while a drop is still forming. They also lose accuracy when drops merge into a continuous stream or when bubbles or condensation scatter light within the chamber.

Optical techniques that attempt to image the entire drip chamber using structured illumination patterns or projected grids are complicated. Pattern-distortion or difference-image methods can improve sensitivity but depend on careful alignment between a patterned backplate and the camera. These systems are often sensitive to ambient light, chamber positioning, and fluid transparency, limiting clinical robustness. Structured illumination also increases manufacturing complexity and calibration time.

Conventional infusion pumps avoid these problems by actively metering flow, but they require motorized syringes or peristaltic mechanisms that add cost, weight, and power consumption. For many low-resource or ambulatory uses, a simpler gravity system with passive flow control remains desirable if comparable accuracy can be achieved.

Accordingly, there remains a need for optically based gravity-infusion systems that can determine instantaneous flow rate during drop formation without relying on projected patterns, discrete drop counting, or external mechanical pumps. There is further need for systems that can automatically regulate or verify flow using linearized multi-lumen inserts and redundant safety valves, while maintaining compliance with medical electrical safety standards. The systems and methods disclosed herein address these other needs.

Nothing in this section should be construed as an admission that any described technique or device is prior art to the present disclosure, and is merely provided to better understand the present disclosure.

### SUMMARY

The present disclosure relates to gravity-driven infusion using image-based regulation. In certain embodiments, a controller may receive a transparent drip chamber of an administration set in a registered pose between a contrasting, preferably infrared-backlit wall and at least one image sensor aligned with an optical path. The transparent drip chamber may be configured for gravity-driven flow in which a fluid forms at least one drop through a spout coupled to and at least partially received within the drip chamber. During setup, the system may perform exposure calibration, verify chamber pose using registration features, test spout geometry for compatibility, and confirm the prime level through a refraction-induced width change visible against the backlit wall. During operation, the system may analyze a spout region of interest for pendant-drop formation and a meniscus region of interest for trend behavior, using optical frames that define a substantially uniform background field to sharpen drop silhouettes while preserving a smooth estimation zone.

The housing may define a monitoring chamber that provides a reproducible mechanical registration for the drip chamber. Registration features such as trident-shaped tines, forks, notches, tabs, or molded ribs may physically index the spout to a fixed measurement location, allowing repeatable positioning. Pixels corresponding to these features may be excluded or dimmed during image analysis. The illumination may include infrared emission, and the image sensor may employ a band-pass filter with emitter timing synchronized to sensor exposure. In some implementations, the illumination may include an illuminable region adjacent to a non-illuminated or opaque region that establishes a silhouette boundary, or may be pulsed, gated, synchronized-continuous, or modulated in intensity and temporally coordinated with camera exposure. The image sensor may be mounted at an oblique angle between approximately five and forty-five degrees relative to a plane normal to the vertical axis of the drip chamber to reduce neck occlusion while maintaining meniscus visibility. In certain embodiments, two cameras may respectively view the spout and meniscus regions with confidence-based data fusion, or an optical element such as a mirror or prism may direct two optical paths to a single sensor for those regions. Multi-camera or stereo configurations may be used for correspondence, rectification, triangulation, and three-dimensional shape reconstruction.

Flow may be estimated across multiple regions of interest, including a spout region where pendant drops form, a meniscus region where level change is monitored, and optionally a lower reservoir impact region capturing ripple or splash signatures that assist in confirming drop detachment or the onset of streaming. The processor may execute a hybrid estimator having several computational tracks. In one track, the system may fit a closed spline around an attached drop boundary using a sparse number of perimeter control points, generally between three and thirty-two, with cubic B-spline interpolation providing smooth curvature. Boundary functionals such as neck span, silhouette area above a chord, vertical centroid, neck arc length, or curvature at a neck saddle may be computed, and their time derivatives may be mapped to instantaneous flow without explicit volume integration while the drop remains attached. Minimal spline configurations may use three anchor points—an apex near the meniscus contact and two at the greatest lateral extent—while compact configurations may use six control points concentrated in the neck region for improved differential stability. The controller may automatically switch between these configurations based on confidence, using minimal settings at high flow rates and compact settings under typical rates.

Machine-learning embodiments may be trained to emit spline control points directly. A composite training loss may include a shape-fidelity term (for example based on symmetric nearest-neighbor or Chamfer distance), an ordering term that preserves boundary traversal direction, and a control-point repulsion term enforcing minimum spacing with region weighting at the neck, where drop detachment timing is most sensitive. A physics-based track may solve pendant-drop Young-Laplace equations using adaptive numerical integration to identify apex pressure that reproduces observed drop width at measured height, generating physically consistent profiles and residuals used for confidence scoring. A baseline-connected variant may determine, within a spout region of interest, a subset of pixels that remain connected to a baseline such as a spout-tangent or meniscus-level line. Morphological opening and closing may remove splash or glare, and instantaneous flow may be estimated from a changing characteristic such as minimal neck width, connected-subset area, vertical centroid, slope of an upper boundary segment, or black-pixel count in a thin neck band. The system may compute a calibrated mapping q_inst(t)=k(C)·dC/dt+b(C), in which k(·) and b(·) are determined from fluid-specific tables stored in a medication library keyed by nominal drop factor, viscosity proxy, surface-tension proxy, and refractive-index proxy. Temporal derivatives may be obtained using finite-difference, Savitzky-Golay, or Kalman filtering, with bounded online adaptation constrained by residuals of the meniscus trend.

A fusion policy may reconcile outputs from two or more estimation tracks such as spline, physics-fit, background-reference, reference-frame subtraction, zero-crossing timing, baseline-connected, and meniscus trend tracks. Weighted averaging or veto logic may be applied based on confidence, edge quality, temporal smoothness, physics residuals, pixel-connectivity stability, and propagated uncertainty. The system may compute propagated uncertainty for each composite estimate and record checksums and manifest identifiers in non-volatile memory for traceability. Drop-period timing from zero crossings of neck width, connected-subset area, or black-pixel count may serve as a supervisory cross-check. The processor may apply rate-dependent low-flow correction, startup gain modifiers at the onset of infusion, and tilt-aware head-height compensation using inertial or posture inputs with observed meniscus drift. Stream detection may involve fitting substantially parallel lateral edges emerging from the spout across consecutive frames, accumulating persistence evidence, and upon exceeding thresholds before a streamed-volume limit, reducing commanded flow and raising a high-priority alarm.

Illumination and sensing may be synchronized so that light surrounding the spout is selectively dimmed during estimation frames to improve silhouette contrast, while other backlit regions remain illuminated for pose verification. Illumination cadence and frame timing may be selected to avoid aliasing with expected drop-formation frequency across the clinical flow range. The system may classify artifacts such as glare, condensation, bubbles, splashes, tubing intrusion, and partial occlusions through spatiotemporal filtering and gradient consistency analysis, ignoring artifacts that fail persistence or spatial-consistency criteria. Adaptive cropping and scaling of the spout region may be used to maintain a target neck-pixel span for reliable measurement.

The system may regulate flow using a motorized flow control valve that compresses a multi-lumen silicone insert positioned downstream of the chamber. The insert may include at least ten lumens and in some embodiments approximately nineteen, providing a substantially linear relationship between displacement and effective cross-sectional area compared to a single-lumen tube. An independent safety occluder valve may serve as a fail-safe closure under fault conditions. A watchdog powered by an internal backup source of about two hundred milliamp-hours may monitor the main power rail and processor heartbeat signals and, upon detecting power loss or inactivity, may automatically close the safety valve and trigger audible and visual alarms. The processor may forecast time-to-event for detachment, stream onset, occlusion, air entrainment, or source exhaustion and may issue pre-alerts within confidence-bounded horizons. Alarm thresholds and escalation pathways may adapt to estimator confidence, artifact persistence, image quality, recent alarm history, and meniscus-trend stability, employing hysteresis and hold-off rules to suppress transient conditions. The controller may also provide advisory outputs suggesting initial rate profiles, head-height adjustments, priming or purging confirmation, chamber cleaning, or ambient-light mitigation, while computing a nuisance-probability score to suppress alerts likely to be false.

Compatible administration sets may include a spike, a transparent drip chamber with an external prime-level indicator, a downstream multi-lumen flow-control insert, a slide clamp interfacing with controller doors to sequentially occlude flow and unlatch doors, and distal luer connectors that meet ISO standards for fourteen- to twenty-two-gauge catheters. The nominal drop factor may be about twenty drops per milliliter. The contrasting wall and drip chamber may define a silhouette frame that enhances edge contrast without requiring structured beams through the fluid. The system may compute difference images and row or column sums to detect free-flow or discrete drop formation, using these results as supervisory checks, and may detect incompatible spouts or unprimed chambers through silhouette or refraction cues. No-flow may be declared by absence of detected drops together with a monotonic meniscus decrease within a time window, and backflow may be declared by absence of drops with a monotonic increase.

A medication library may store fluid parameters and infusion limits to configure estimators and control settings. A communications interface may receive prescription updates or infusion parameters from authenticated information systems, compare received data to the current programmed state, alert users to discrepancies, and upon clinician confirmation reconfigure control parameters, adjust estimator priors, and record updates with timestamps and operator credentials. Unsigned or unauthenticated commands may be rejected. All computations may preserve integrity through checksum verification and manifest tracking.

The controller may include a processor, memory storing executable instructions, and an image-sensor interface configured to receive spout and meniscus images. The processor may compute a composite flow estimate by combining a spline-derived non-volume track and a meniscus-trend track, and may generate control signals to drive a flow control valve and a safety occluder valve. The controller may select mapping coefficients from the medication library, compute physics references and residuals as confidence inputs, adapt spline-control density within the neck region to preserve monotonic measures during growth, and perform pre-infusion checks including exposure calibration, pose verification, spout compatibility, prime-level verification by refraction, and opaque-fluid detection. The controller may implement a streaming detector that accumulates lateral-edge evidence and escalates alarms when persistence exceeds a set limit before an estimated streamed-volume threshold, and may maintain a spout locator initialized from a first reliably segmented drop and re-localized when deviation exceeds a defined threshold. The flow-control valve may exhibit a substantially linear area-displacement characteristic across its operating range. A non-transitory computer-readable medium may store instructions that, when executed by such a controller, cause it to perform spline fitting, compute boundary functionals and their derivatives, compute meniscus trends, fuse estimates, select mapping coefficients from fluid-aware tables, perform bounded online adaptation using meniscus residuals, compute Young-Laplace physics references, gate illumination, log propagated uncertainty, classify artifacts, and escalate alarms based on persistence and estimated streamed volume.

In multi-source or “piggyback” arrangements, two or more gravity controllers may merge flows upstream of a patient line. Each controller may include the foregoing chamber, wall, image sensor, and processor. The combined system may include a junction merging outflows and an orchestration processor that enforces a programmed delivery schedule, applies virtual head-height equalization biases computed as effective hydrostatic pressure offsets per line to maintain commanded patient-level flow substantially independent of relative bag heights, verifies closures before handoffs, and monitors each controller for backflow or streaming. A piggyback mode may transition a first line to maintenance or closed state, open a secondary line to a target rate, perform a defined flush, and resume the first line without manual height adjustments. The orchestration processor may reject unsigned or unauthenticated schedule or rate commands, log authenticated updates with timestamps and operator credentials, veto handoffs when ripple or stream signatures are detected in a third region of a line proposed to open or close, and coordinate alarms such that a persistent anomaly in one line escalates a shared alarm while maintaining or safely reducing delivery through the other. A corresponding method of orchestration may include computing per-line composite flow estimates, allocating per-line setpoints to satisfy a patient-level rate, compensating relative hydrostatic head differences without manual bag-height adjustment, and executing verified-closure handoffs with optional flush intervals, as well as rejecting openings when ripple or stream signatures indicate conflicting flow and logging authenticated schedule updates.

Baseline-based embodiments may evaluate boundary functionals relative to a baseline defined by a spout-tangent line at a neck anchor or by a meniscus-level line in a lower-reservoir coordinate frame, using truncated silhouette area above the baseline and vertical centroid relative to the baseline as instantaneous flow indicators. Baseline position may be verified during pre-infusion by a refraction-induced width change at a known backlit bar within a prime-level window, and the baseline may be fixed in controller coordinates constrained by mechanical pose features. Connectivity-enabled variants may determine baseline-connected pixel subsets, estimate drop characteristics from those subsets while the drop remains attached, and compute real-time flow from temporal changes in those characteristics without explicit volume computation, applying calibrated mappings q=k(C)·dC/dt+b(C) and differentiating C(t) by finite-difference, Savitzky-Golay, or Kalman filtering. A confidence module may down-weight or veto instantaneous flow estimates when the connected subset is intermittent or violates spatial-consistency constraints, reverting to zero-crossing or meniscus-trend estimation. Illumination cadence and frame timing may be selected to avoid aliasing with expected drop-formation frequency, and fiducial elements near the spout may be temporarily dimmed during estimation frames. Automatic switching between three-point and six-point spline configurations may occur based on real-time confidence values.

The described systems, methods, controllers, and computer-readable media may provide image-based monitoring and regulation of gravity-driven infusion through real-time optical analysis of drop formation and meniscus trends, hybrid non-volume estimation with physics-consistent validation, connectivity-aware and baseline-referenced alternatives, confidence-weighted data fusion, adaptive illumination, and multi-source orchestration. Embodiments may include a multi-lumen actuation scheme and independent safety mechanisms enforced by watchdog circuitry to achieve controlled and fail-safe infusion delivery. The described techniques may be implemented in hardware, firmware, or software recorded on computer-readable storage media configured to cause the system to perform the actions described.

In an embodiment, the administration set may include a downstream tube segment that incorporates an anti-pinch member at a location intended for pinching or clamping. This anti-pinch member is defined by a second portion having a length less than the first portion of the tube and is configured to inhibit localized point contacts within the tube's lumen. This specific construction ensures that, when a compressive force is applied, the relationship between restriction and applied force is comparatively more linear than a tube configuration lacking the anti-pinch member. In a further embodiment, this anti-pinch member may comprise a sleeve, shell, or bonded insert made of a polymer that provides greater hoop stiffness than the base tube, and it features tapered ends to reduce stress concentration. The system may be configured so that a flow control valve acts directly across the anti-pinch member, and a multi-lumen insert may be disposed immediately adjacent to it, such that valve displacement over an operating range produces a substantially linear effective area change. The anti-pinch member may be positioned at the same pinch site as the multi-lumen insert or at a secondary clamp site, where its function is to prevent the formation of localized point contacts during periods of partial occlusion. The system may additionally include a tube-restoring mechanism selected from several options, including opposed flexible strips that actively round the tube, a fluid-based bladder using elastomeric fillers to support and restore the tube's shape, or a geared restorative assembly designed to compress over-expanded wall portions to re-round the tube. The verification of this tube restoration may be accomplished through an optical meniscus-drift check at a zero-valve command or by employing a calibrated low-rate displacement-versus-flow test.

Furthermore, the system may utilize a dual-mode backlight incorporating a first diffuser field that is substantially uniform within a spout Region of Interest (ROI) for boundary estimation and a second diffuser field that emits a striped pattern used to detect continuous fluid streams. The processing of difference images using row or column sums may serve as a supervisory indicator to monitor free flow or discrete drop formation, allowing alarms to be gated when a set threshold is exceeded. A method of regulation may further comprise applying the anti-pinch member to a downstream tube portion at a pinch site to inhibit point contacts and to linearize restriction under applied force during the regulation process. In an embodiment, first and second cameras respectively oriented toward the spout and lower impact ROIs, with per-view estimates combined according to confidence; having two cameras may facilitate determining both drop formation/evolution/growth and when a drop detaches from the spout and puddles.

A gravity-driven infusion system may regulate flow by visually monitoring a transparent drip chamber and automatically commanding a pinch mechanism on a multi-lumen insert. A compact controller mounts adjacent the chamber and presents a contrasting, infrared-backlit wall to a camera aligned with the chamber's optical path. A motorized flow-control valve acts on the multi-lumen insert to meter flow while an independent safety occluder, supervised by a watchdog with backup power, provides fail-safe shutoff. The controller continuously estimates and regulates flow based on image features rather than pump displacement, enabling closed-loop control of manual gravity infusions.

The vision subsystem may define at least two regions of interest (ROIs): a spout ROI that captures the attached pendant drop and a meniscus ROI that captures the slower reservoir-level drift. The controller may mount the image sensor at an oblique angle between about 5° and 45° relative to a plane normal to the chamber axis to optimize contrast and avoid reflections, and it can employ two cameras, e.g., one may be biased to the spout ROI and one to the lower reservoir, or an optical element that provides two paths to a single sensor. A spout locator initializes from the first reliably segmented drop, maintains position with a recursive filter, and re-localizes if deviation exceeds a threshold. Exposure and illumination are coordinated so the emitter is temporally gated to sensor integration; during estimation frames, a localized zone around the spout can be dimmed or blanked while other silhouette features remain visible for pose verification. The backlight can operate in dual modes: a uniform diffuser field that supports boundary estimation in the spout ROI and a striped or patterned field that enhances detection of continuous streaming.

Flow may be estimated without relying solely on explicit volume integration of a reconstructed drop. While a drop remains attached, the controller fits a compact set of perimeter spline control points to the drop silhouette and computes boundary-based functionals-such as neck width, truncated silhouette area above a baseline chord, and vertical centroid-whose temporal changes map directly to instantaneous flow. A baseline for these calculations can be defined by a spout-tangent line at a neck anchor or by a meniscus level in a lower-reservoir coordinate frame, with baseline registration verified by refraction-induced width change at a known backlit bar within a prime-level window. A physics reference, for example a Young-Laplace pendant-drop solution, may be solved and compared to image features so that residuals contribute a confidence signal. The controller fuses per-track estimates—e.g., spline track, physics-fit track, meniscus trend, background-reference, reference-frame subtraction, or zero-crossing timing—into a composite flow estimate with confidence weighting. For traceability, the system computes propagated uncertainty, logs checksums and manifest identifiers with each estimate, and stores these records in non-volatile memory.

Supervisory detectors may corroborate and guard the estimator. Difference images with row- or column-sums provide a lightweight channel to distinguish free flow from discrete drop formation and can gate alarms. Streaming detection can be performed by fitting substantially parallel lateral edges emerging from the spout across frames and accumulating persistence evidence; when persistence exceeds a threshold, the controller reduces commanded flow and escalates to a high-priority alarm before a streamed-volume limit is reached. A third ROI in a lower reservoir impact region can detect ripple or splash to validate detachment timing and to veto inaccurate timing cues during transitions. Artifact classification (e.g., glare, condensation, bubbles, splash ejecta, tubing intrusion, partial occlusions) suppresses short-lived or spatially inconsistent signatures, improving robustness.

Pre-infusion checks may include exposure calibration, mechanical pose registration of the chamber (so spout and fiducials are in repeatable locations), spout-geometry compatibility, prime-level verification by refraction-induced width change, and opaque-fluid detection. During operation the controller may adaptively crop or scale the spout ROI to maintain a target pixel span at the neck and sample the meniscus ROI between drops. Alarm policy can incorporate estimator confidence, artifact persistence, recent alarm history, and meniscus-trend stability, with hysteresis and holdoff to reduce nuisance alerts while preserving safety.

The mechanical flow interface of the administration set which may placed between the pinch heads/valves of the controller during use, may use a multi-lumen insert positioned between independently actuated pinch heads: the primary flow-control valve and the safety occluder. Multiple lumens provide a near-linear relation between valve displacement and effective cross-sectional area compared with single-lumen tubing, improving controllability at low and moderate occlusions. To preserve tubing geometry over time, the system may apply an anti-pinch member at the pinch site to inhibit point contacts and linearize restriction under force, and it may periodically actuate a tube-restoring mechanism to reduce ovalization; restoration can be verified by a zero-command meniscus-drift check or a calibrated low-rate displacement-versus-flow test. A watchdog, powered by a backup source, can autonomously command the safety occluder closed and sustain a secondary alarm upon loss of a main power rail or processor heartbeat.

The controller may be tilt-aware and head-height-aware, and may adjust its measurements and/or estimates by compensating for any such tilt or other environmental factors by utilizing a physics based model. Relative hydrostatic effects may be compensated using inertial or posture inputs together with observed meniscus drift so that commanded rate remains stable despite changes in bag elevation or patient posture. Low-rate behavior can be further improved with rate-dependent correction and a startup gain modifier that compensates for flow decay at commencement of infusion.

Configuration and connectivity features support safe clinical workflows. A medication library may store fluid parameters and infusion constraints (e.g., nominal drop factor, viscosity and surface-tension proxies, refractive-index proxies) that select mapping coefficients and bound online adaptation using meniscus-trend residuals. A communications interface can receive authenticated prescription or schedule updates; unsigned or unauthenticated instructions are rejected. Upon confirmed updates, the controller reconfigures control parameters, adjusts estimator priors, and records timestamps and operator credentials. The controller can forecast time-to-event for drop detachment, stream onset, occlusion, air entrainment, or source exhaustion, issuing pre-alerts within a bounded horizon; advisory insights may include initial rate or ramp suggestions, head-height guidance, line-purge confirmation, chamber-clear instructions, and ambient-light mitigation, with a nuisance-likelihood score that suppresses low-value notifications.

A multi-source orchestration variant coordinates two or more gravity-driven sources upstream of a common patient line. An orchestration processor enforces a delivery schedule, applies virtual head-height equalization biases—implemented as effective hydrostatic pressure offsets per line—to maintain a commanded patient-level rate independent of relative bag heights, verifies closures before handoffs, and monitors each source for backflow or streaming. A piggyback mode transitions a primary line to maintenance or closed, opens a secondary line to a target rate, optionally performs a defined flush, and then resumes the primary line without manual height adjustments. Handoffs can be vetoed if ripple or stream signatures are detected in the third ROI of a line proposed to open or close; alarm policies can be coordinated so that a persistent anomaly in one line escalates a shared alarm while maintaining or safely reducing delivery from a healthy line.

When instantaneous volume is needed, in some embodiments, the imaged drop may be be axially sliced using calibrated pixel area or interpreted as a surface of revolution around a vertical axis; the derivative dV/dt provides instantaneous flow, and these volumetric paths are reconciled with the boundary-functional tracks within the same fusion framework. All estimates, confidence measures, configuration identifiers, and event logs are persisted to support post-hoc analysis and regulatory traceability. In embodiments, a direct drop volume calculation may not be needed as other factors such as fluid characteristics and observed characteristics of the drop, such as pattern recognition from trained data or a determination of drop height without needing to perform any integration or volume calculation would yield an accurate determination of drop volume.

These and other aspects of the present disclosure may become more apparent from the following detailed description of the various embodiments when taken in conjunction with the accompanying drawings. Further features and advantages of the invention may become apparent from the following detailed description taken in conjunction with the accompanying figures showing illustrative embodiments, in which like reference numerals designate like elements throughout the different views.

### BRIEF DESCRIPTION OF THE DRAWINGS

These and other aspects will become more apparent from the following detailed description of the various embodiments of the present disclosure with reference to the drawings wherein:

FIG. 1 shows a block diagram of a system for regulating fluid flow in accordance with an embodiment of the present disclosure;

FIG. 2 shows a flowchart diagram of a method for exposing an image sensor in accordance with an embodiment of the present disclosure;

FIG. 3 shows a timing diagram illustrating an embodiment of the method of FIG. 2 in accordance with an embodiment of the present disclosure;

FIGS. 4A-4B show illustrations of image data (i.e., images) captured by a flow meter of a drip chamber to illustrate an embodiment of the method for exposing an image sensor of FIG. 2 in accordance with the timing diagram of FIG. 3 in accordance with an embodiment of the present disclosure;

FIG. 5 shows a diagram of a flow meter and valve that are integrated together for coupling to a drip chamber and an IV bag in accordance with an embodiment of the present disclosure;

FIG. 6 is a block diagram of an imaging system of a flow meter for imaging a drip chamber in accordance with an embodiment of the present disclosure;

FIG. 7 is a graphic illustration of an image captured by the image sensor of the system of FIG. 6 in accordance with an embodiment of the present disclosure;

FIG. 8 is a block diagram of an imaging system of a flow meter for imaging a drip chamber utilizing a background pattern in accordance with an embodiment of the present disclosure;

FIG. 9 is a graphic illustration of an image captured by an image sensor of a flow meter disclosed herein when a free flow condition exists in accordance with an embodiment of the present disclosure;

FIG. 10 is a graphic illustration of an image captured by an image sensor of a flow meter for use as a background image in accordance with an embodiment of the present disclosure;

FIG. 11 is a graphic illustration of an image captured by an image sensor when drops are being formed within a drip chamber in accordance with an embodiment of the present disclosure;

FIG. 12 is a graphic illustration of an image captured by an image sensor for use as a background image in accordance with an embodiment of the present disclosure;

FIG. 13 is a graphic illustration of a difference between the images of FIGS. 11 and 12 with additional processing in accordance with an embodiment of the present disclosure;

FIG. 14 is a graphic representation of some of the image processing performed using FIGS. 11-13 to determine if a free flow condition exists in accordance with an embodiment of the present disclosure;

FIG. 15 is a graphic illustration of an image captured by the image sensor when a free flow condition exists in accordance with an embodiment of the present disclosure;

FIG. 16 is a graphic illustration of an image captured by the image sensor for use as a background image in accordance with an embodiment of the present disclosure;

FIG. 17 is a graphic illustration of a difference between the images of FIGS. 15 and 16 with some additional processing for use in detecting a free flow condition in accordance with an embodiment of the present disclosure;

FIG. 18 is a graphic representation of some of the image processing performed using FIGS. 15-17 to determine if a free flow condition exists in accordance with an embodiment of the present disclosure;

FIG. 19 illustrates a template for pattern matching to determine if a free flow condition exits in accordance with an embodiment of the present disclosure;

FIG. 20 is a graphic illustration of a difference between a reference image and an image containing a stream processed with edge detection and line detection for use in detecting a free flow condition in accordance with an embodiment of the present disclosure;

FIG. 21 is a graphic illustration of an image of a drip chamber captured by an image sensor when a free flow condition exists in accordance with an embodiment of the present disclosure;

FIG. 22 is a block diagram of an imaging system for use with a flow meter having a background pattern with stripes and a light source shining on the stripes from an adjacent location to an image sensor in accordance with an embodiment of the present disclosure;

FIG. 23 is a block diagram of an imaging system for use with a flow meter having a background pattern with stripes and a light source shining on the stripes from behind the background pattern relative to an opposite end of an image sensor in accordance with an embodiment of the present disclosure;

FIG. 24 illustrates an image from an image sensor when a drop distorts the background pattern of FIG. 23 in accordance with an embodiment of the present disclosure;

FIG. 25 is a block diagram of an imaging system for use with a flow meter having a background pattern with a checkerboard pattern and a light source shining on the stripes from behind the background pattern relative to an opposite end of an image sensor in accordance with an embodiment of the present disclosure;

FIG. 26 shows an image from the image sensor of FIG. 25 when a drop distorts the background pattern in accordance with an embodiment of the present disclosure;

FIGS. 27-28 show a flow chart illustration of a method for estimating a volume of a drop within a drip chamber in accordance with an embodiment of the present disclosure;

FIGS. 29-31 illustrate images used or generated by a flow meter to estimate a volume of a drop within a drip chamber using the method illustrated by FIGS. 27-28 in accordance with an embodiment of the present disclosure;

FIG. 32 shows pseudo code for identifying a plurality of pixels of interest in accordance with the method of FIGS. 27-28 in accordance with an embodiment of the present disclosure;

FIGS. 33-36 illustrate additional images used or generated by a flow meter to estimate a volume of a drop within a drip chamber using the method illustrated by FIGS. 27-28 in accordance with an embodiment of the present disclosure;

FIG. 37 shows pseudo code for determining a subset of pixels within the plurality of pixels of interest that corresponds to a drop in accordance with an embodiment of the present disclosure;

FIG. 38 shows a ray diagram illustrating the diameter of a blur circle to illustrate aspects of an image sensor of an imaging system disclosed herein in accordance with an embodiment of the present disclosure;

FIG. 39 is a graphic illustrating a calculated blur circle for a variety of lens-to-focal plane separations and lens-to-image separations for an image sensor of an imaging system disclosed herein in accordance with an embodiment of the present disclosure;

FIG. 40 is a graphic illustrating a blur circle divided by a pixel size when a 20 millimeter focal length lens of an image sensor of an imaging system disclosed herein is used in accordance with an embodiment of the present disclosure;

FIG. 41 is a graphic illustrating a blur circle divided by a pixel size when a 40 millimeter focal length lens of an image sensor of an imaging system disclosed herein is used in accordance with an embodiment of the present disclosure;

FIG. 42 shows a table illustrating the corresponding fields of view about the optical axis for the corners of two configurations of an imaging system disclosed herein in accordance with an embodiment of the present disclosure;

FIG. 43 shows a flow meter coupled to a drip chamber in accordance with an embodiment of the present disclosure;

FIG. 44 shows the flow meter and drip chamber of FIG. 43 with the door open in accordance with an embodiment of the present disclosure;

FIG. 45 shows a flow meter coupled to a drip chamber in accordance with an embodiment of the present disclosure;

FIG. 46 shows a flow meter and a pinch valve coupled to the body of the flow meter to control the flow of fluid into a patient in accordance with an embodiment of the present disclosure;

FIG. 47 shows a close-up view of the pinch valve that is coupled to the body of the flow meter of FIG. 46 in accordance with an embodiment of the present disclosure;

FIG. 48 shows a flow meter and a pinch valve wherein the flow meter includes two image sensors in accordance with another embodiment of the present disclosure;

FIG. 49 shows a flow meter and a valve including two curved, elongated support members to control the flow of fluid into a patient in accordance with an embodiment of the present disclosure;

FIGS. 50A-50B show close-up views of the valve of FIG. 49 in accordance with an embodiment of the present disclosure;

FIGS. 51A-51D show several views of a flow meter with a monitoring client, a valve, a drip chamber, an IV bag and a fluid tube wherein the flow meter includes a receiving portion to receive the valve in accordance with an embodiment of the present disclosure;

FIGS. 52A-52D show several views of another flow meter with a valve, a drip chamber, and a tube wherein the flow meter has a receiving portion to receive the valve in accordance with an embodiment of the present disclosure;

FIG. 53A shows another view of the valve of FIGS. 51A-51D and 52A-52D in accordance with an embodiment of the present disclosure;

FIGS. 53B-53C show two exploded views of the valve of FIG. 53A in accordance with an embodiment of the present disclosure;

FIG. 54 shows the valve of FIG. 53 in manual use in accordance with an embodiment of the present disclosure;

FIG. 55 shows a valve that includes two flexible members in accordance with an embodiment of the present disclosure;

FIGS. 56A-56C show several views of a valve having two curved, elongated support members with one of the elongated support members having a plurality of ridges adapted to engage a tube in accordance with an embodiment of the present disclosure;

FIGS. 57A-57C show several views of a valve having a ratchet that engages a connecting member in accordance with an embodiment of the present disclosure;

FIGS. 57D-57E show two exploded views of the valve of FIGS. 57A-57C in accordance with an embodiment of the present disclosure;

FIGS. 58A-58D show several views of a valve having two elongated support members, a connecting member, and a screw-type actuator in accordance with another embodiment of the present disclosure;

FIGS. 59A-59C show several views of a body of a valve in accordance with an embodiment of the present disclosure;

FIGS. 59D-590 show several views of a knob for use with the body shown in FIGS. 59A-59C in accordance with an embodiment of the present disclosure;

FIG. 59H shows the assembled valve that includes the body shown in FIGS. 59A-59C coupled to the knob of FIGS. 59D-59G in accordance with an embodiment of the present disclosure;

FIG. 60 shows a valve having a guiding protrusion in accordance with an embodiment of the present disclosure;

FIG. 61 shows a motor and a valve-securing structure for coupling to the valve of FIG. 60 in accordance with an embodiment of the present disclosure;

FIG. 62 shows the valve of FIG. 60 secured to the motor and the valve-securing structure of FIG. 61 in accordance with an embodiment of the present disclosure;

FIG. 63 shows another motor and valve-securing structure for coupling to the valve of FIG. 60 in accordance with an embodiment of the present disclosure;

FIG. 64A shows a valve having a collar and several fingers for regulating fluid flow through a fluid line in accordance with an embodiment of the present disclosure;

FIG. 648 shows a cross-sectional view of the valve of FIG. 64A in accordance with an embodiment of the present disclosure;

FIG. 65 shows a cross-sectional view of a valve having two curved surfaces for positioning a fluid tube therebetween to regulate fluid flow through the fluid tube in accordance with an embodiment of the present disclosure;

FIGS. 66A-660 show several views of a valve having a knob to move a connecting member which is locked into position after movement of the knob in accordance with an embodiment of the present disclosure;

FIG. 67 shows a graphic that illustrates actuation vs. flow rates for a valve in accordance with an embodiment of the present disclosure;

FIG. 68A shows a flow meter that uses binary optics in accordance with an embodiment of the present disclosure;

FIG. 68B shows the circuit for use with FIG. 68A in accordance with an embodiment of the present disclosure;

FIGS. 69A-69F show several views of a safety valve that may be used with a flow meter in accordance with an embodiment of the present disclosure;

FIG. 70 shows a flow chart diagram illustrating a method of estimating drop growth and/or flow within a drip chamber in accordance with an embodiment of the present disclosure;

FIGS. 71A-71E show images taken by a flow meter with a template overlaid therein to illustrate the method of FIG. 70 in accordance with an embodiment of the present disclosure;

FIG. 72 shows a modulatable backlight assembly in accordance with an embodiment of the present disclosure;

FIGS. 73A-73C show several views of a tube-restoring apparatus in accordance with an embodiment of the present disclosure;

FIG. 74 shows a system for regulating fluid flow using a valve having two flexible strips in accordance with an embodiment of the present disclosure;

FIG. 75 shows the valve of FIG. 74 in accordance with an embodiment of the present disclosure;

FIG. 76A shows a valve that utilizes a fluid-based bladder in accordance with an embodiment of the present disclosure;

FIG. 76B shows a cross-sectional view of the assembled valve of FIG. 76A with two elastomeric fillers in accordance with an embodiment of the present disclosure;

FIG. 77 shows a system for regulating fluid flow using a valve having two flexible strips actuatable by a linear actuator in accordance with an embodiment of the present disclosure;

FIG. 78 shows the system of FIG. 77 with the valve actuated in accordance with an embodiment of the present disclosure;

FIG. 79 shows a close-up view of the valve of FIGS. 77-78 in accordance with an embodiment of the present disclosure;

FIG. 80 shows a close-up view of the valve as actuated in FIG. 78 in accordance with an embodiment of the present disclosure;

FIG. 81 shows several images for use to illustrate a method of estimating drop growth and/or fluid flow illustrated in FIGS. 82A-82B in accordance with an embodiment of the present disclosure; and

FIGS. 82A-82B show a flow chart diagram illustrating a method of estimating drop growth and/or fluid flow in accordance with an embodiment of the present disclosure; and

FIG. 83 shows a flow chart diagram of a method for reducing noise from condensation in accordance with an embodiment of the present disclosure.

FIG. 84 is a perspective view of an infusion system including an infusion controller and a compatible administration set.

FIGS. 85A-85E include orthogonal views of the infusion controller including front, back (with door), bottom, and left/right views and showing the clinician-facing display, keypad, status light, window to the monitoring chamber, and the rear door that grants access to the drip-chamber cavity.

FIG. 86 is a schematic of the administration set (illustrative, not to scale) including a vented spike, drip chamber with spout, multi-lumen flow-control insert, roller clamp, slide clamp, tubing, and a distal luer connector.

FIG. 87 illustrates integration of the administration set of FIG. 86 with the infusion controller of FIG. 84, including the seated drip chamber, the monitoring cavity, and alignment of the multi-lumen insert between the flow-control (FCV) and safety-occluder (SOV) pinch heads.

FIG. 88 illustrates insertion of the slide clamp into a slide-clamp slot on the controller to occlude tubing prior to door release.

FIG. 89 illustrates loading the primed administration-set drip chamber into the controller's cavity and closing the door to establish a defined optical pose and guide the flow-control insert between the FCV and SOV.

FIG. 90 depicts example display parameters of the infusion controller, including rate, VTBI, time remaining, and a fully populated icon tray.

FIG. 91 provides additional exterior views (a back-perspective with the door open, a right-side view, and a front view) highlighting the drip-chamber cavity, backlit wall, and clinician interface.

FIG. 92 illustrates a representative step in coupling the administration set to the controller, including positioning of the drip chamber and routing of the flow-control insert.

FIG. 93 illustrates priming of the drip chamber to a prime-level ring to enable optical monitoring while mitigating air-infusion risk.

FIG. 94 illustrates successive cross-sections of the multi-lumen flow-control insert as it is compressed between valve pinch heads and a backstop.

FIG. 95 illustrates steps showing that insertion of the slide clamp into the slot first occludes the administration-set tubing and then opens the controller door.

FIG. 96 is an exploded perspective of the administration set showing principal components and material interfaces.

FIG. 97 is a system-architecture diagram of the infusion controller highlighting dual processor cores (Linux/vision and real-time/control), camera and illumination drivers, valve actuation, power sources (main and backup), and a watchdog circuit. Components powered by the backup source are indicated.

FIG. 98 illustrates a contrasting, backlit pattern as viewed with no drip chamber installed (left) and with a properly primed drip chamber installed (right), indicating representative regions of interest (ROIs).

FIG. 99 shows camera images used for empty and loaded fiducial checks that verify correct chamber loading and pose.

FIG. 100 illustrates prime-level ROIs showing under-primed, over-primed, and correctly primed states identified via refraction-induced width change.

FIG. 101 outlines active-infusion ROIs used by the vision system for drop detection, stream detection, and meniscus tracking.

FIG. 102 illustrates frame-by-frame camera measurements of a pendant drop forming at the spout.

FIG. 103 illustrates the image-processing stages from a raw frame to a drop-edge map, splash mask, and resulting drop mask.

FIG. 104 illustrates the drop mask and the intersection-voting analysis used to localize a drop's center and leading edge.

FIG. 105 is a chart showing drop-level and black-pixel-count signals characteristic of drop growth and detachment.

FIG. 106 illustrates vision-system identification of a continuous-stream condition using lateral-edge evidence and confidence accumulation.

FIG. 107 presents control-system mapping of detected drop level (left) to estimated drop volume (right) at a programmed 300 mL/hr rate.

FIG. 108 is a graph of totaled drop-volume data used in rate estimation (example at 300 mL/hr).

FIG. 109 shows per-point weights applied in the linear-regression rate computation (example at 300 mL/hr).

FIG. 110 illustrates a weighted-regression fit and the corresponding real-time flow-rate estimate (example at 300 mL/hr).

FIG. 111 shows zero-crossing analyses of drop-level (top) and black-pixel-count (bottom) signals, with estimated means shown as red lines.

### DETAILED DESCRIPTION

The embodiments described herein relate to monitoring and regulating gravity-driven infusion using optical sensing of a transparent drip chamber and digital control of downstream flow restriction. Unless otherwise indicated, terms such as “controller,” “processor,” “camera,” “image sensor,” “contrasting wall,” “backlight,” “flow-control valve” (FCV), “safety occluder valve” (SOV), “administration set,” “spout,” and “meniscus” correspond to the elements recited in the claims. “Background field” denotes a visually contrasting background—e.g., a back-lit or otherwise contrasting wall—that provides sufficient intensity contrast for boundary detection in the optical path. “Region of interest” (ROI) refers to a sub-image window such as a spout ROI or meniscus ROI. Optional and alternative features may be used in any operable combination.

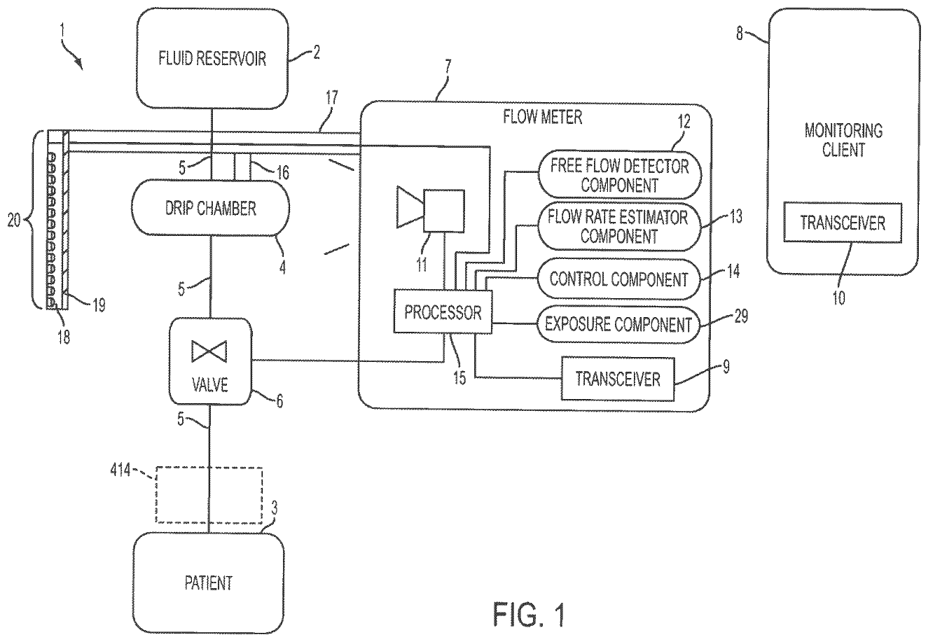

FIG. 1 shows a block diagram of a system 1 for regulating fluid flow in accordance with an embodiment of the present disclosure. For example, system 1 may regulate, monitor, and/or control the flow of fluid into a patient 3. The system 1 includes a fluid reservoir 2 for infusing fluid contained therein into the patient 3. The fluid reservoir 2 is gravity fed into a drip chamber 4 via a fluid tube 5. The fluid reservoir 2, the drip chamber 4, and the patient 3 may be considered as part of the system 1 or may be considered as separate or optional work pieces for the system 1 (e.g., any fluid reservoir 2 and drip chamber 4 may be used to treat any patient 3).

A flow meter 7 monitors the drip chamber 4 to estimate a flow rate of liquid flowing through the drip chamber 4. The fluid from the drip chamber 4 is gravity fed into a valve 6. The valve 6 regulates (i.e., varies) the flow of fluid from the fluid reservoir 2 to the patient 3 by regulating fluid flow from the drip chamber 4 to the patient 3. The valve 6 may be any valve as described herein, including a valve having two curved-shaped members, a valve having two flexible sheets, a valve that pinches (or uniformly compresses) on the tube over a significant length of the tube, or the like. The valve 6 may be an inverse-Bourdon-tube valve that works in an opposite way of a Bourdon tube in that a deformation of the fluid path causes changes in fluid flow rather than fluid flow causing deformation of the fluid path.

In alternative embodiments, the system 1 optionally includes an infusion pump 414 (e.g., a peristaltic pump, a finger pump, a linear peristaltic pump, a rotary peristaltic pump, a cassette-based pump, a membrane pump, other pump, etc.) coupled to the fluid tube 5. The outlined box designated as 414 represents the optional nature of the infusion pump 414, e.g., the infusion pump may not be used in some embodiments. The infusion pump 414 may use the flow meter 7 as feedback to control the flow of fluid through the fluid tube 5. The infusion pump 414 may be in wireless communication with the flow meter 7 to receive the flow rate therefrom. The infusion pump 414 may use a feedback control algorithm (e.g., the control component 14 of FIG. 1) to adjust the flow of fluid, such as a proportional-integral-derivative (“PID”), bang-bang, neural network, and/or fuzzy logic control system. In this specific exemplary embodiment (i.e., an embodiment having the infusion pump 414), the valve 6 is optional. However, in other embodiments, the valve 6 may or may not be used, and/or is optional. The infusion pump 414 may adjust the rotation of a cam and/or a motor in accordance with measurements from the flow meter 7, such as flow rate, volume infused, total volume infused, etc. Additionally or alternatively, the infusion pump 414 may stop fluid flow (e.g., by stopping the pumping action) when the flow meter 7 communicates to the infusion pump 414 that a free flow condition exists. In yet additional embodiments, the monitoring client 8 controls the operation of the infusion pump 414 (e.g., via a wireless connection) and receives feedback from the flow meter 7.

In some embodiments, the fluid reservoir 2 is pressurized to facilitate the flow of fluid from the fluid reservoir 2 into the patient 3, e.g., in the case where the fluid reservoir 2 (e.g., an IV bag) is below the patient 3; The pressurization provides sufficient mechanical energy to cause the fluid to flow into the patient 3. A variety of pressure sources, such as physical pressure, mechanical pressure, and pneumatic pressure may be applied to the inside or outside of the fluid reservoir 2. In one such embodiment, the pressurization may be provided by a rubber band wrapped around an IV bag.

The flow meter 7 and the valve 6 may form a closed-loop system to regulate fluid flow to the patient 3. For example, the flow meter 7 may receive a target flow rate from a monitoring client 8 by communication using transceivers 9, 10. That is, the transceivers 9, 10 may be used for communication between the flow meter 7 and the monitoring client 8. The transceivers 9, 10 may communicate between each other using a modulated signal to encode various types of information such as digital data or an analog signal. Some modulation techniques used may include using carrier frequency with FM modulation, using AM modulation, using digital modulation, using analog modulation, or the like.

The flow meter 7 estimates the flow rate through the drip chamber 4 and adjusts the valve 6 to achieve the target flow rate received from the monitoring client 8. The valve 6 may be controlled by the flow meter 7 directly from communication lines coupled to an actuator of the valve 6 or via a wireless link from the flow meter 7 to onboard circuitry of the valve 6. The onboard electronics of the valve 6 may be used to control actuation of the valve 6 via an actuator coupled thereto. This closed-loop embodiment of the flow meter 7 and the valve 6 may utilize any control algorithm including a PID control algorithm, a neural network control algorithm, a fuzzy-logic control algorithm, the like, or some combination thereof.

The flow meter 7 is coupled to a support member 17 that is coupled to the drip chamber 4 via a coupler 16. The support member 17 also supports a backlight 18. The backlight 18 includes an array of LEDs 20 that provides illumination to the flow meter 7. In some specific embodiments, the backlight 18 includes a background pattern 19. In other embodiments, the backlight 18 does not include the background pattern 19. In some embodiments, the background pattern 19 is present in only the lower portion of the backlight 18 and there is no background pattern 19 on the top (e.g., away from the ground) of the backlight 18.

The flow meter 7 includes an image sensor 11, a free flow detector component 12, a flow rate estimator component 13, a control component 14, an exposure component 29, a processor 15, and a transceiver 9. The flow meter 7 may be battery operated, may be powered by an AC outlet, may include supercapacitors, and may include on-board, power-supply circuitry (not explicitly shown).

The image sensor 11 may be a CCD sensor, a CMOS sensor, or other image sensor. The image sensor 11 captures images of the drip chamber 4 and communicates image data corresponding to the captured images to the processor 15.

The processor 15 is also coupled to the free flow detector component 12, the flow rate estimator component 13, the control component 14, and the exposure component 29. The free flow detector component 12, the flow rate estimator component 13, the control component 14, and the exposure component 29 may be implemented as processor-executable instructions that are executable by the processor 15 and may be stored in memory, such as a non-transitory, processor-readable memory, ROM, RAM, EEPROM, a harddisk, a harddrive, a flashdrive, and the like.

The processor 15 can execute the instructions of the free flow detector component 12 to determine if a free flow condition exists within the drip chamber 4 by analyzing the image data from the image sensor 11. Various embodiments of the free flow detector component 12 for detecting a free flow condition are described below. In response to a detected free flow condition, the processor 15 can make a function call to the control component 14 to send a signal to the valve 6 to completely stop fluid flow to the patient 3. That is, if the free flow detector component 12 determines that a free flow condition exists, the flow meter 7 may instruct the valve 6 to stop fluid flow, may instruct the monitoring client 8 to stop fluid flow (which may communicate with the valve 6 or the pump 414), and/or may instruct the pump 414 to stop pumping or occlude fluid flow using an internal safety occluder.

The flow rate estimator component 13 estimates the flow rate of fluid flowing through the drip chamber 4 using the image data from the image sensor 11. The processor 15 communicates the estimated flow rate to the control component 14 (e.g., via a function call). Various embodiments of estimating the flow rate are described below. If the flow rate estimator component 13 determines that the flow rate is greater than a predetermined threshold or is outside a predetermined range, the flow meter 7 may instruct the valve 6 to stop fluid flow (which may communicate with the valve 6 or the pump 414), may instruct the monitoring client 8 to stop fluid flow (which may communicate with the valve 6 or the pump 414), and/or may instruct the pump 414 to stop pumping or occlude fluid flow using an internal safety occluder.

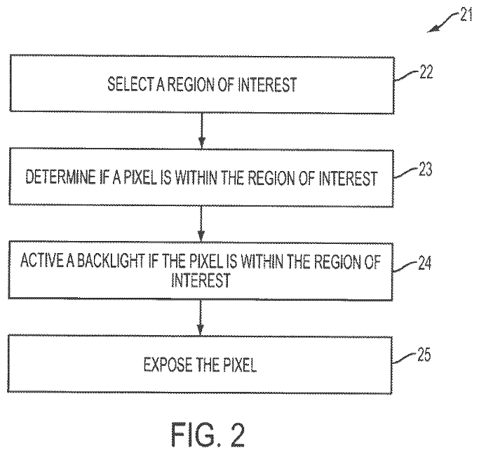

The processor 15 controls the array of LEDs 20 to provide sufficient light for the image sensor 11. For example, the exposure component 29 may be used by the processor 15 or in conjunction therewith to control the array of LEDs 20 such that the image sensor 11 captures image data sufficient for use by the free flow detector component 12 and the flow rate estimator component 13. The processor 15 may implement an exposure algorithm stored by the exposure component 29 (see FIG. 2) to control the lighting conditions and/or the exposure of the image sensor 11 when generating the image data. Additionally or alternatively, the exposure component 29 may be implemented as a circuit, an integrated circuit, a CPLD, a PAL, a PLD, a hardware-description-language-based implementation, and/or a software system.

The control component 14 calculates adjustments to make to the valve 6 in accordance with the estimated flow rate from the flow rate estimator component 13. For example and as previously mentioned, the control component 14 may implement a PID control algorithm to adjust the valve 6 to achieve the target flow rate.

The monitoring client 8, in some embodiments, monitors operation of the system 1. For example, when a free flow condition is detected by the free flow detector component 12, the monitoring client 8 may wirelessly communicate a signal to the valve 6 to interrupt fluid flow to the patient 3.

The flow meter 7 may additionally include various input/output devices to facilitate patient safety, such as various scanners, and may utilize the transceiver 9 to communicate with electronic medical records, drug error reduction systems, and/or facility services, such as inventory control systems.

In a specific exemplary embodiment, the flow meter 7 has a scanner, such as an RFID interrogator that interrogates an RFID tag attached to the fluid reservoir 2 or a barcode scanner that scans a barcode of the fluid reservoir 2. The scanner may be used to determine whether the correct fluid is within the fluid reservoir 2, it is the correct fluid reservoir 2, the treatment programmed into the flow meter 7 corresponds to the fluid within the fluid reservoir 2 and/or the fluid reservoir 2 and flow meter 7 are correct for the particular patient (e.g., as determined from a patient's barcode, a patient's RFID tag, or other patient identification).

For example, the flow meter 7 may scan the RFID tag of the fluid reservoir 2 to determine if a serial number or fluid type encoded within the RFID tag is the same as indicated by the programmed treatment stored within the flow meter 7. Additionally or alternatively, the flow meter 7 may interrogate the REID tag of the fluid reservoir 2 for a serial number and the RFID tag of the patient 3 for a patient serial number, and also interrogate the electronic medical records using the transceiver 9 to determine if the serial number of the fluid reservoir 2 within the RFID tag attached to the fluid reservoir 2 matches the patient's serial number within the RFID tag attached to the patient 3 as indicated by the electronic medical records.

Additionally or alternatively, the monitoring client 8 may scan the RFID tag of the fluid reservoir 2 and the RFID tag of the patient 3 to determine that it is the correct fluid within the fluid reservoir 2, it is the correct fluid reservoir 2, the treatment programmed into the flow meter 7 corresponds to the fluid within the fluid reservoir 2, and/or the fluid reservoir 2 is correct for the particular patient (e.g., as determined from a patient's barcode, RFID tag, electronic medical records, or other patient identification or information). Additionally or alternatively, the monitoring client 8 or the flow meter 7 may interrogate the electronic medical records database and/or the pharmacy to verify the prescription or to download the prescription, e.g., using the serial number of the barcode on the fluid reservoir 2 or the RFID tag attached to the fluid reservoir 2.

FIG. 2 shows a flow chart diagram of a method 21 for exposing an image sensor, e.g., the image sensor 11 of FIG. 1, in accordance with an embodiment of the present disclosure. The method 21 includes acts 22, 23, 24, and 25. Method 21 may be implemented by the processor 15 of FIG. 1 (e.g., as the exposure component 29) and may be implemented as a processor-implemented method, as a set of instructions configured for execution by one or more processors, in hardware, in software, the like, or some combination thereof.

Act 22 selects a region of interest. For example, referring again to FIG. 1, the image sensor 11 includes a field of view that includes the drip chamber 4. However, the drip chamber 4 may not occupy the entire field of view of the image sensor 11. Act 22 selects only the pixels of the image sensor 11 that show, for example, the drip chamber 4.

Act 23 determines if a pixel is within the region of interest 23. If the pixel of act 23 is a pixel that images, for example, the drip chamber 4, then act 23 determines that it is within the region of interest. Likewise, in this example, if the pixel of act 23 is a pixel that does not image the drip chamber 4, act 23 determines that the pixel is not within the region of interest.

Act 24 activates a backlight, e.g., the backlight 18 of FIG. 1, if the pixel is within the region of interest. Pixels of an image sensor may be exposed during different times. Thus, the backlight 18 may be activated only when pixels within the region of interest are being exposed. For example, some image sensors include vertical and horizontal sync signals. The backlight may be synchronized with these signals to turn on when a pixel of interest is being exposed.

In some embodiments of the present disclosure, a subset of LEDs of the backlight (e.g., a subset of the LED array 20, which may be a 2-dimensional array) may be turned on. The subset may be a sufficient subset to sufficiently illuminate the pixel being exposed if the pixel is within the region of interest.

Act 25 exposes the pixel. If in act 23 it was determined that the pixel is within the region of interest, the pixel will be exposed with at least a portion of the backlight turned on in act 25. Additionally, if in act 23 it was determined that the pixel is not within the region of interest, the pixel will be exposed without at least a portion of the backlight turned on in act 25.

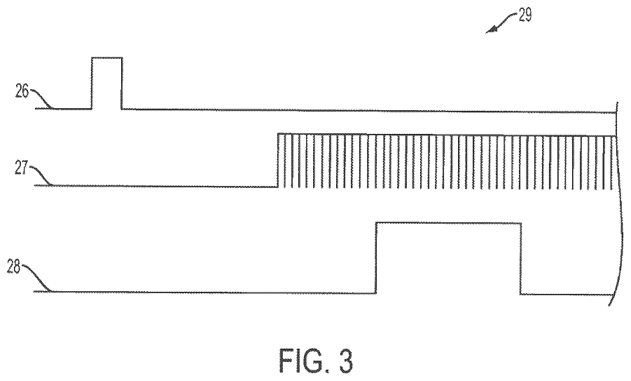

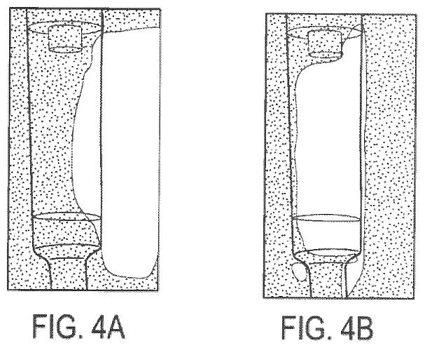

FIG. 3 shows a timing diagram 29 illustrating an embodiment of the method of FIG. 2 in accordance with an embodiment of the present disclosure. The timing diagram 29 includes traces 26, 27, and 28. Trace 26 is a vertical sync signal from an image sensor and trace 27 is a horizontal sync signal from the image sensor (e.g., image sensor 11 of FIG. 1). A circuit or software routine (e.g., the exposure component 29 found in the flow meter 7 of FIG. 1) may use the sync traces 26, 27 to generate a backlight-enable signal 28 that is used to activate a backlight or a subset thereof.

FIGS. 4A-4B show illustrations of image data of a flow meter 7 illustrating an embodiment of the method of FIG. 2 in accordance with the timing diagram of FIG. 3 in accordance with an embodiment of the present disclosure. FIG. 4A illustrates the image data taken by a flow meter, such as the flow meter 7 of FIG. 1, without the use of the exposure algorithm illustrated in FIGS. 2 and 3; FIG. 4B illustrates the image data taken by the flow meter with the use of the exposure algorithm illustrated in FIGS. 2 and 3. Less power is needed to provide illumination during the capture of the image of FIG. 4B than to provide illumination for the capture of the image of FIG. 4A because of less use of the backlight.

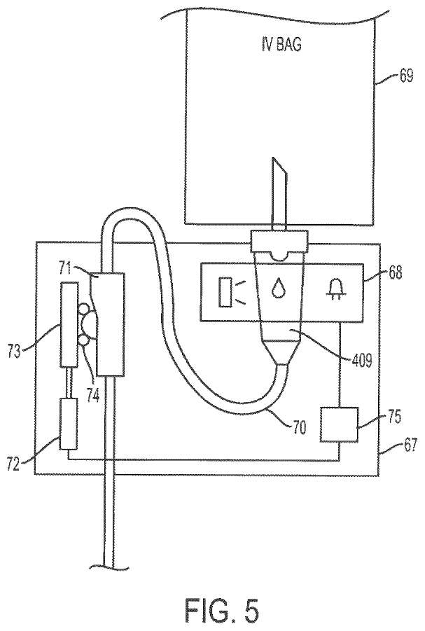

FIG. 5 shows a diagram of a flow meter 67 and a valve 71 that are integrated together for coupling to a drip chamber 409 and an IV bag 69 in accordance with an embodiment of the present disclosure. The flow meter 67 includes an optical drip counter 68 that receives fluid from the IV bag 69. The optical drip counter 68 may be an image sensor, a pair of image sensors, a capacitive drip counter, and/or the like. The flow meter 67 is coupled to a tube 70 coupled to a roller clamp 71 that is controlled by a motor 72. The motor 72 is coupled to a lead screw mechanism 73 to control a roller clamp 71 via interaction with interacting members 74.

The motor 72 may be a servo motor and may be used to adjust the flow rate through the tube 70. That is, the flow meter 67 may also function as a flow meter and regulator. For example, a processor 75 within the flow meter 67 may adjust the motor 72 such that a desired flow rate is achieved as measured by the optical drip counter 68. The processor 75 may implement a control algorithm using the optical drip counter 68 as feedback, e.g., a PID control loop with the output supplied to the motor 72 and the feedback received from the optical drip counter 68.

In alternative embodiments, the motor 72, the lead screw mechanism 73, and the roller clamp 71 may be replaced and/or supplemented by an actuator that squeezes the tube 70 (e.g., using a cam mechanism or linkage driven by a motor) or they may be replaced by any sufficient roller, screw, or slider driven by a motor. For example, in some embodiments of the present disclosure, the roller clamp 71 may be replaced by any valve as described herein, including a valve having two C-shaped members, a valve having two curve-shaped support members, a valve having two flexible sheets, a valve that pinches on the tube over a significant length of the tube, or the like.

The flow meter 67 may also optionally include a display. The display may be used to set the target flow rate, display the current flow rate, and/or provide a button, e.g., a touch screen button to stop the flow rate.

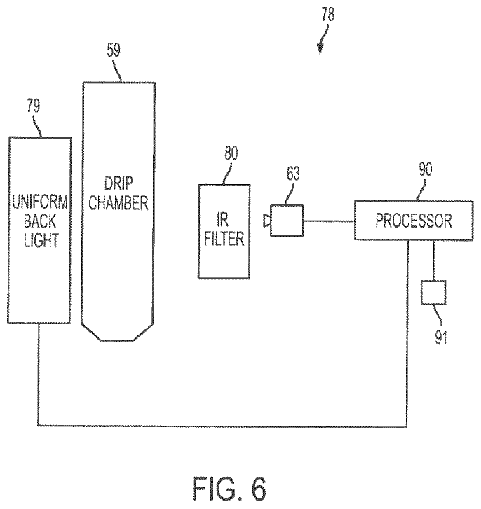

FIG. 6 is a block diagram of an imaging system 78 of a flow meter for imaging a drip chamber in accordance with an embodiment of the present disclosure. The imaging system 78 as shown in FIG. 6 may be used within any flow meter described herein, including the flow meter 7 of FIG. 1 and/or the flow meter 67 of FIG. 5.

The imaging system 78 of FIG. 6 includes an image sensor 63, a uniform backlight 79 to shine light at least partially through the drip chamber 59, and an infrared (“IR”) filter 80 that receives the light from the uniform backlight 79.

System 78 also includes a processor 90 that may be operatively coupled to the image sensor 63 and/or the uniform backlight 79. The processor 90 implements an algorithm to determine when a free flow condition exists and/or to estimate a flow rate (e.g., using the free flow detector component 12 or the flow rate estimator component 13 of FIG. 1). The processor 90 may be in operative communication with a processor-readable memory 91 (e.g., a non-transitory, processor-readable memory) to receive one or more instructions to implement the algorithm to determine if a free flow condition exists and/or to estimate the flow rate. The one or more instructions from the processor-readable memory 91 are configured for execution by the processor 90.

The uniform backlight 79 may be an array of light-emitting diodes (“LEDs”) having the same or different colors, a light bulb, a window to receive ambient light, an incandescent light, and the like. In some embodiments, the uniform backlight 79 may include one or mom point-source lights.

The processor 90 may modulate the uniform backlight 79 in accordance with the image sensor 63. For example, the processor 90 may activate the uniform backlight 79 for a predetermined amount of time and signal the image sensor 63 to capture at least one image, and thereafter signal the uniform backlight 79 to turn off. The one or more images from the image sensor 63 may be processed by the processor 90 to estimate the flow rate and/or detect free flow conditions. For example, in one embodiment of the present disclosure, the system 78 monitors the size of the drops being formed within the drip chamber 59, and counts the number of drops that flow through the drip chamber 59 within a predetermined amount of time; the processor 90 may average the periodic flow from the individual drops over a period of time to estimate the flow rate. For example, if X drops each having a volume Y flow through the drip chamber in a time Z, the flow rate may be calculated as (X*Y)/Z.

Additionally or alternatively, the system 78 may determine when the IV fluid is streaming through the drip chamber 59 (i.e., during a free flow condition). The uniform backlight 79 shines light through the drip chamber 59 to provide sufficient illumination for the image sensor 63 to image the drip chamber 59. The image sensor 63 can capture one or more images of the drip chamber 59.

Other orientations and configurations of the system 78 may be used to account for the orientation and output characteristics of the uniform backlight 79, the sensitivity and orientation of the image sensor 63, and the ambient light conditions. In some embodiments of the present disclosure, the processor 90 implements an algorithm that utilizes a uniformity of the images collected by the image sensor 63. The uniformity may be facilitated by the uniform backlight 79. For example, consistent uniform images may be captured by the image sensor 63 when a uniform backlight 79 is utilized.

Ambient lighting may cause inconsistencies in the images received from the image sensor 63; for example, direct solar illumination provides inconsistent lighting because the sun may be intermittently obscured by clouds and the sun's brightness and angle of illumination depend upon the time of the day. Therefore, in some embodiments of the present disclosure, an IR filter 80 is optionally used to filter out some of the ambient light to mitigate variations in the images captured by the image sensor 63. The IR filter 80 may be a narrow-band infrared light filter placed in front of the image sensor 63; and the uniform backlight 79 may emit light that is about the same wavelength as the center frequency of the passband of the filter 80. The IR filter 80 and the uniform backlight 79 may have a center frequency of about 850 nanometers. In some embodiments, the imaging system 78 may be surrounded by a visually translucent, but IR-blocking, shell. In alternative embodiments, other optical frequencies, bandwidths, center frequencies, or filter types may be utilized in the system 78.

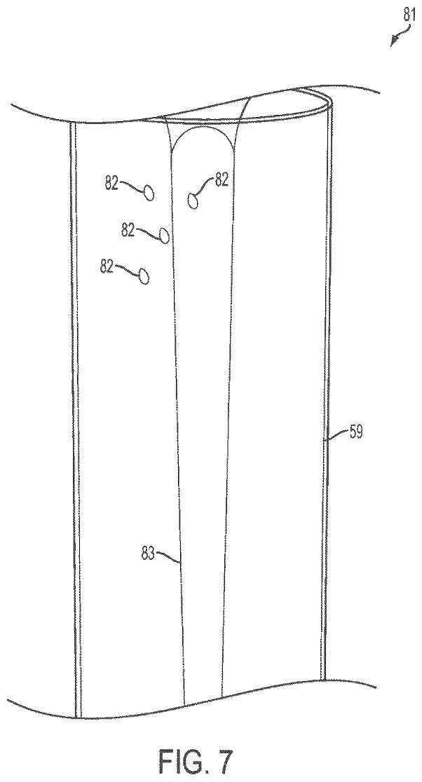

FIG. 7 is a graphic illustration of an image 81 captured by the image sensor 63 of the system 78 of FIG. 6 in accordance with an embodiment of the present disclosure. The image 81 is an image of a drip chamber 59 having condensation 82 and a stream 83 caused by a free flow condition therein. Edge detection may be used to determine the position of the stream 83 and/or the condensation 82, in some embodiments. Additionally or alternatively, a background image or pattern may be used.

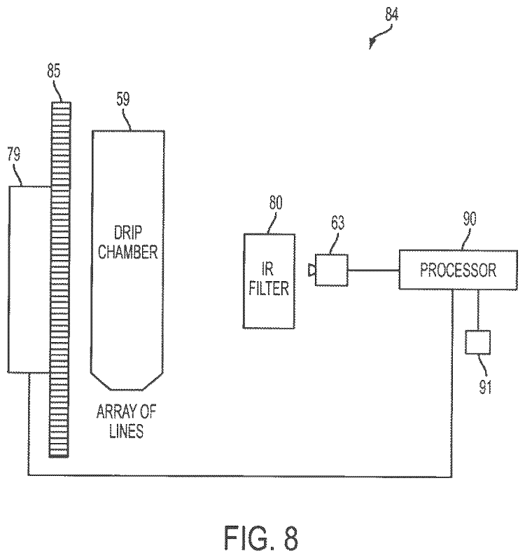

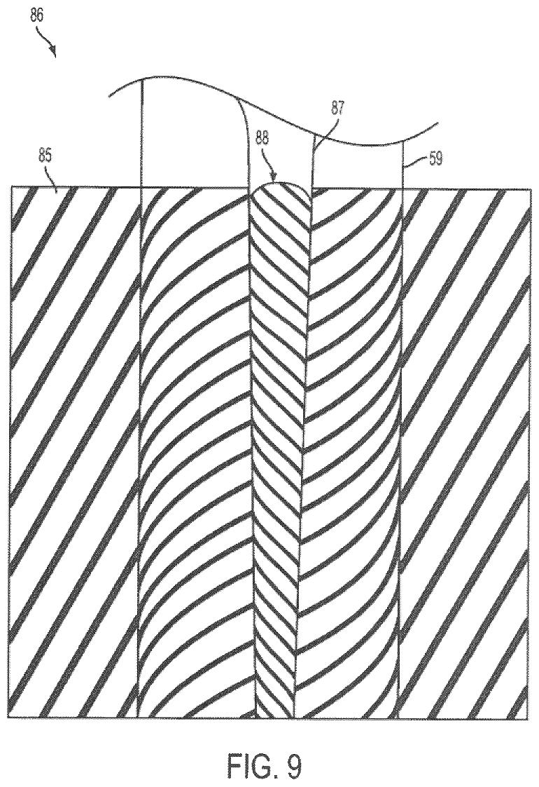

FIG. 8 is a block diagram of an imaging system 84 of a flow meter for imaging a drip chamber in accordance with an embodiment of the present disclosure. The imaging system 84 may be used with any flow meter disclosed herein, including the flow meter 7 of FIG. 1 and the flow meter 67 of FIG. 5.

System 84 includes an array of lines 85 that are opaque behind the drip chamber 59. System 84 uses the array of lines 85 to detect a free flow condition. The free flow detection algorithm (e.g., the free flow detector component 12 of FIG. 1) may use the presence or absence of drops for determining whether or not a streaming condition (e.g., a free flow condition) exists.

In some specific embodiments, the lines 85 are only present on a fraction of the image (e.g., the background pattern only occupies a fraction of the backlight 18 or the binary optics only causes the pattern to appear in a fraction of the image, such as the lower or upper half). For example, a lower fraction of the image may include a background pattern of stripes.

Referring now to FIG. 9, a graphic illustration of an image 86 is shown as captured by the image sensor 63 of FIG. 8 when a free flow condition exists in the drip chamber 59 in accordance with an embodiment of the present disclosure. The image 86 illustrates the condition in which the drip chamber 59 experiences a free flow condition and illustrates the effect that the stream of fluid 87 acts as a positive cylindrical lens. That is, as shown in FIG. 9, the array of lines 85 as captured in an image by the image sensor 63 are shown as a reversed line pattern 88 from the array of lines 85 as compared to a non-free flow condition. The appearance of the reversed line pattern 88 is caused by changes to the light when the light passes through the stream of fluid 87 as the light approaches the image sensor 63.

In some embodiments of the present disclosure, illumination by light having an optical wavelength of about 850 nanometers may be used to create the image 86. Some materials may be opaque in the visible spectrum and transparent in the near IR spectrum at about 850 nanometers and therefore may be used to create the array of lines 85. The array of lines 85 may be created using various rapid-prototyping plastics. For example, the array of lines 85 may be created using a rapid-prototype structure printed with an infrared-opaque ink or coated with a metal for making the array of lines 85. Additionally or alternatively, in some embodiments of the present disclosure, another method of creating the array of lines 85 is to create a circuit board with the lines laid down in copper. In another embodiment, the array of lines 85 is created by laying a piece of ribbon cable on the uniform backlight 79; the wires in the ribbon cable are opaque to the infrared spectrum, but the insulation is transparent such that the spacing of the wires may form the line for use during imaging by the image sensor 63 (see FIG. 8). In yet additional embodiments, a piece of thin EDMed metal may be utilized. Metal is opaque to light and the spaces between the metal material deposits may be very finely controlled during manufacture to allow the IR light to pass through the spaces.

The processor 90 implements an algorithm to determine when a free flow condition exists (e.g., using the free flow detector component 12 of FIG. 1). The processor 90 may be in operative communication with a processor-readable memory 91 (e.g., a non-transitory, processor-readable memory) to receive one or more instructions to implement the algorithm to determine if a free flow condition exists. The one or more instructions from the processor-readable memory 91 are configured for execution by the processor 90.

Referring again to FIG. 8, blood may be used by the system 84. For example, system 84 may determine when a free flow condition of blood exists when utilizing the image sensor 63, the IR filter 80, and the uniform backlight 79 configured, for example, for use using optical light having a wavelength of 850 nanometers or 780 nanometers, e.g., when using bovine blood. The blood may appear opaque compared to the images taken using water.

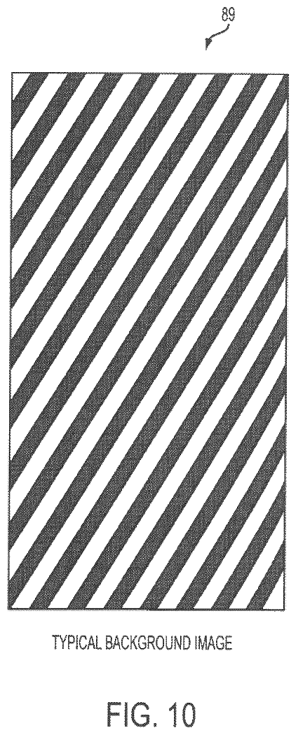

The following algorithm implemented by the processor 90 and received from the processor-readable memory 91 may be used to determine when a free flow condition exists: (1) establish a background image 89 (see FIG. 10); and (2) subtract the background image 89 from the current image. Additionally processing may be performed on the resulting image.

In some embodiments of the present disclosure, the background image 89 of FIG. 10 may be dynamically generated by the processor 90. The dynamic background image may be used to account for changing conditions, e.g. condensation or splashes 82 on the surface of the drip chamber 59 (see FIG. 7). For example, in one specific embodiment, for each new image captured by the image sensor (e.g., 63 of FIG. 8), the background image has each pixel multiplied by 0.96 and the current image (e.g., the most recently captured image) has a respective pixel multiplied by 0.04, after which the two values are added together to create a new value for a new background image for that respective pixel; this process may be repeated for all of the pixels. In yet another example, in one specific embodiment, if a pixel of the new image is at a row, x, and at a column, y, the new background image at row, x, and column, y, is the value of the previous background image at row, x, and column, y, multiplied by 0.96, which is added to the value of the pixel at row, x, and column, y of the new image multiplied by 0.04.

When the system 84 has no water flowing through the drip chamber 59 (see FIG. 8) the resulting subtraction should be almost completely back, i.e., low pixel magnitudes, thereby facilitating the algorithm to determine that the drip chamber 59 has no water flowing therethrough.

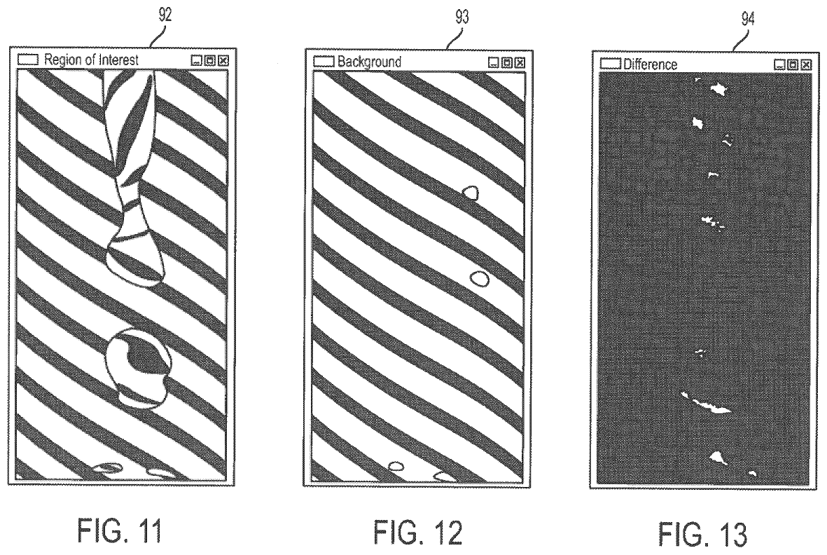

FIG. 11 shows an image 92 from the image sensor 63 when there is a drop within the drip chamber 59 (see FIG. 8). FIG. 12 shows a background image 93 used by the system 84. When the system 84 has a drop as shown in image 92 of FIG. 11, the system 84 of FIG. 8 has a few high contrast-spots where the image of the array of lines is warped by the lensing of the droplet as illustrated by an image 94 of FIG. 13. Image 94 of FIG. 13 is generated by taking, for each respective pixel, the absolute value of the subtraction of the image 92 of FIG. 11 from image 93 of FIG. 12, and converting each respective pixel to a white pixel if the value is above a predetermined threshold or otherwise converting the pixel to a black pixel when the value is below the predetermined threshold. Each white pixel within the image 94 of FIG. 13 is a result of there being a difference for that pixel location between the images 92 and 93 that is greater than a predetermined threshold.

For example, consider three respective pixels of FIGS. 11, 12, and 13 having a location of row x and column y. To determine the pixel of row x and column y for the image 94 of FIG. 13, the pixel at row x and column y of image 92 of FIG. 11 is subtracted from the pixel at row x and column y of image 93 of FIG. 12, then the absolute value of the result of the subtraction is taken; and if the absolute value of the result is above a predetermined threshold (e.g., above a grayscale value of 128, for example), the pixel at the location of row x and column y of image 94 of FIG. 13 is white, otherwise the pixel at the location of row x and column y of image 94 of FIG. 13 is black.

When it is determined that a few high-contrast spots exist within the image 94 of FIG. 13, the processor 90 of system 84 (see FIG. 8) determines that drops are being formed within the drip chamber 59 and no free flow condition exists. The images of the drops may be utilized to determine the size of the drops to estimate a flow rate as described herein.

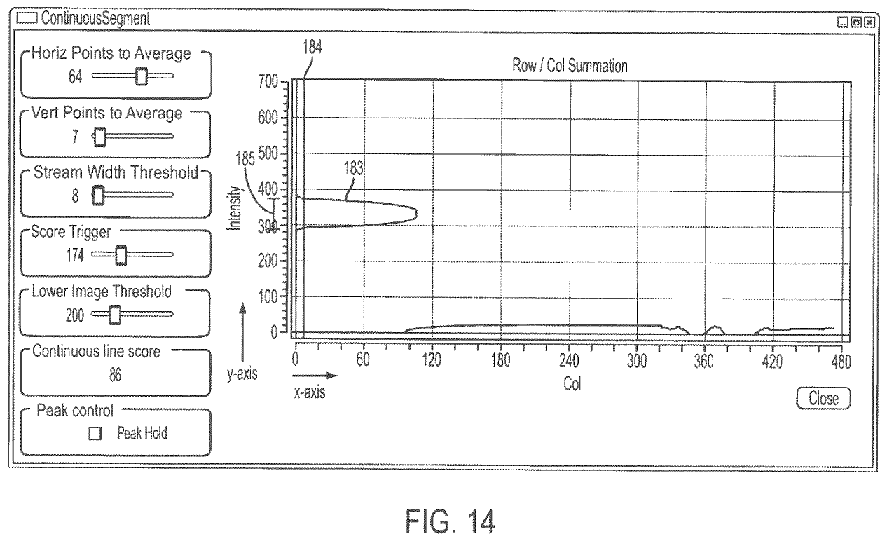

FIG. 14 is a graphic representation of some of the image processing that may be performed using FIGS. 11-13 to determine if a free flow condition exists in accordance with an embodiment of the present disclosure. Referring to FIGS. 14 and 13, all of the white pixels for each row are summed together, and are illustrated in FIG. 14 as results 183. The y-axis represents the row number, and the x-axis represents the summed number of white pixels for each respective row.

Referring now to only FIG. 14, as previously mentioned, the number of white pixels for each row is summed together and is illustrated as results 183, which are used to determine if or when a free flow condition exists. In some specific embodiments, the processor 90 of system 84 (see FIG. 8) determines that a free flow condition exists when a predetermined number of contiguous values of the summed rows of the results 183 exists above a threshold 184. For example, within the results 183, a range of a plurality of contiguous rows represented generally by 185 has a total value above the threshold 184. When greater than a predetermined number of contiguous summed rows is determined to exist within the results 183 above a predetermined threshold (e.g., threshold 184), a free flow condition is determined to exist by the processor 90 of FIG. 8. For example, as shown in FIG. 14, the range of the plurality of contiguous summed rows 185 is below the predetermined number of contiguous summed rows (i.e., the range 185 is not wide enough) and therefore a free flow condition is determined to not exist.

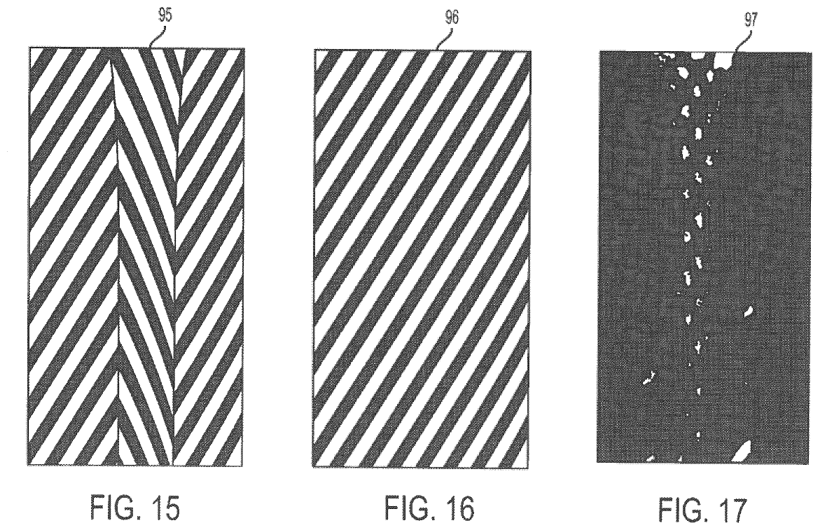

FIG. 15 shows an image 95 showing a stream as captured by the image sensor 63 of FIG. 8 when a free flow condition exists. FIG. 16 shows a background image 96. FIG. 17 shows an image 97 formed by the absolute value of the difference between the image 96 of FIG. 16 and the image 95 from FIG. 15 when the absolute value is converted either to a white pixel (when the absolute value of the difference is above a threshold) or to a black pixel (when the absolute value of the difference is below the threshold). As shown in FIG. 17, high-contrast spots caused by the reverse orientation of the lines in the stream that run from top to bottom are detectable by the processor 90. The processor 90 of FIG. 8 can use the image 97 to determine if a free flow condition exists using the algorithm described above.

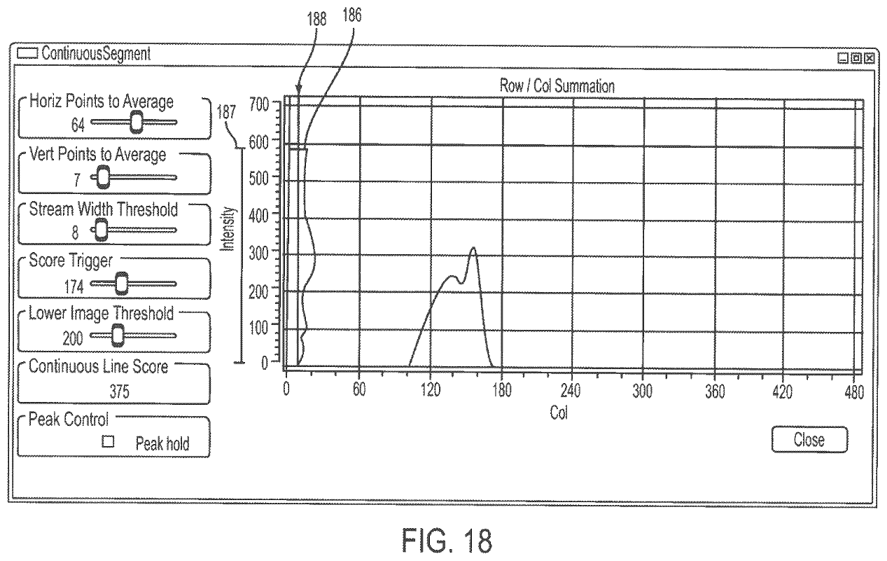

That is, as shown in FIG. 18, results 186 are shown as having a contiguous range 187 of the results 186 that are above a threshold 188. Because the contiguous range 187 of summed rows is greater than a predetermined threshold number of contiguous values above the threshold 188, a free flow condition is determined to exist by the processor 90 (see FIG. 8). That is, the contiguous range of the results 186 above the threshold 188 is greater than a predetermined threshold range of contiguous values; therefore, the processor 90 determines that a free flow condition exists when using the results 186 of FIG. 18.

In yet an additional embodiment of the present disclosure, the intensity, the intensity squared, or other function may be used to produce the results 183 of FIG. 14 and/or the results 186 of FIG. 18. In yet an additional embodiment, one or more data smoothing functions may be used to smooth the results 183 and/or 186, such as a spline function, a cubic spline function, a B-spline function, a Bezier spline function, a polynomial interpolation, a moving average, or other data smoothing function.

For example, an image of the image sensor 63 of FIG. 8, e.g., image 95 of FIG. 15, may be subtracted from a background image, e.g., the image % of FIG. 16, to obtain intensity values. That is, a pixel of row x and column y of FIG. 15 may be subtracted from a pixel of row x and column y of the image 96 of FIG. 16 to create an intensity value at row x and column y; this may be repeated for all pixel locations to obtain all of the intensity values. The intensity values of each row may be summed together to obtain the results 183 and/or 186 (see FIGS. 14 and 18, respectively), such that the processor 90 may determine that a free flow condition exists when the summed rows of the intensity values has a contiguous range of summed rows above a threshold. In some embodiments, the intensity values are converted to absolute values of the intensity values, and the summed rows of the absolute values of the intensity values are used to determine if a contiguous range of summed rows of the absolute values is above a threshold range of contiguous values. Additionally or alternatively, the intensity may be squared and then the processor 90 may sum the squared intensity rows and determine if a contiguous range of summed rows of the intensity squared values exists beyond a threshold range of contiguous values to determine if a free flow condition exists.

In some embodiments, a predetermined range of contiguous values above a threshold (e.g., min and max ranges) of the summed rows of intensity values or intensity squared values may be used by the processor 90 to determine if a drop of liquid is within the image. For example, each row of the rows of the intensity values (or the intensity squared values) may be summed together and a range of the summed values may be above a threshold number; if the range of contiguous values is between a minimum range and a maximum range, the processor 90 may determine that the range of contiguous values above a predetermined threshold is from a drop within the field of view of the image sensor 63 (see FIG. 8). In some embodiments of the present disclosure, the summed rows of intensity values or intensity squared values may be normalized, e.g., normalized to have a value between 0 and 1.

The following describes a smoothing function similar to the cubic spline (i.e., the cubic-spline-type function) that may be used on the summed rows, the summed rows of intensity values, or the summed rows of the intensity values squared prior to the determination by the processor 90 to determine if a free flow condition exits. In some specific embodiments, the cubic-spline-type function may be used to identify blocks, as described infra, which may facilitate the processor's 90 identification of free flow conditions.

Equation Chapter (Next) Section 1 The cubic-spline-type function is an analog to the cubic spline, but it smoothes a data set rather than faithfully mimics a given function. Having data sampled on the interval from [0,1] (e.g., the summation along a row of intensity squared or intensity that is normalized) the processor 90 (see FIG. 6 or 8) may find the best fit set of cubic functions on the intervals [x₀,x₁], [x₁, x₂], . . . [x_{N-1},x_{N}] with x₀=0 and x_{N}=1 where the total function is continuous with continuous derivatives and continuous curvature.

The standard cubic spline definition is illustrated in Equation (1) as follows:

$$\begin{matrix}χ ( x ) = {A}_{i} ( x ) {y}_{i} + {B}_{i} ( x ) {y}_{i + 1} + {C}_{i} ( x ) {y}_{i}^{″} + {D}_{i} ( x ) {y}_{i + 1}^{″} & {x}_{i} \leq x \leq {x}_{i + 1}\end{matrix} , \qquad ( 1 )$$

with the functions B C, D, defined as in the set of Equations (2):

$$\begin{matrix}{A}_{i} ( x ) = \frac{{x}_{i + 1} - x}{{x}_{i + 1} - {x}_{i}} = \frac{{x}_{i + 1} - x}{{Δ}_{i}} , & {B}_{i} = \frac{x - {x}_{i}}{{x}_{i + 1} - {x}_{i}} = \frac{x - {x}_{i}}{{Δ}_{i}} \\ {C}_{i} ( x ) = \frac{{Δ}_{i}^{2}}{6} ( {A}_{i}^{3} ( x ) - {A}_{i} ( x ) ) , & {D}_{i} = \frac{{Δ}_{i}^{2}}{6} ( {B}_{i}^{3} ( x ) - {B}_{i} ( x ) )\end{matrix} . \qquad ( 2 )$$

The Equations (1) and (2) guaranty continuity and curvature continuity. The only values which can be freely chosen are yi,

$${y}_{0}^{″} and {y}_{N}^{″} .$$

Please note that Equation (3) is chosen as follows:

$${y}_{0}^{″} = {y}_{1}^{″} = 0 , \qquad ( 3 )$$

i.e., the function is flat at 0 and 1. The remaining

$${y}_{1}^{″}$$

must satisfy the following set of Equations (4):

$$\begin{matrix}\frac{{y}_{1} - {y}_{0}}{{Δ}_{0}} + \frac{{y}_{1}^{″} {Δ}_{0}}{3} = \frac{{y}_{2} - {y}_{1}}{{Δ}_{1}} - \frac{{y}_{1}^{″} {Δ}_{1}}{3} - \frac{{y}_{2}^{″} {Δ}_{1}}{6} \\ \frac{{y}_{2} - {y}_{1}}{{Δ}_{1}} + \frac{{y}_{1}^{″} {Δ}_{1}}{6} + \frac{{y}_{2}^{″} {Δ}_{1}}{3} = \frac{{y}_{3} - {y}_{2}}{{Δ}_{2}} - \frac{{y}_{2}^{″} {Δ}_{2}}{3} - \frac{{y}_{3}^{″} {Δ}_{2}}{6} \\ \frac{{y}_{3} - {y}_{2}}{{Δ}_{2}} + \frac{{y}_{2}^{″} {Δ}_{2}}{6} + \frac{{y}_{3}^{″} {Δ}_{2}}{3} = \frac{{y}_{4} - {y}_{3}}{{Δ}_{3}} - \frac{{y}_{3}^{″} {Δ}_{3}}{3} - \frac{{y}_{4}^{″} {Δ}_{3}}{6} \\ ⋮ \\ \frac{{y}_{N - 2} - {y}_{N - 3}}{{Δ}_{N - 3}} + \frac{{y}_{N - 2}^{″} {Δ}_{N - 3}}{6} + \frac{{y}_{N - 2}^{″} {Δ}_{N - 3}}{3} = \frac{{y}_{N - 1} - {y}_{N - 2}}{{Δ}_{N - 2}} - \frac{{y}_{N - 2}^{″} {Δ}_{N - 2}}{3} - \frac{{y}_{N - 1}^{″} {Δ}_{N - 2}}{6} \\ \frac{{y}_{N - 1} - {y}_{N - 2}}{{Δ}_{N - 2}} + \frac{{y}_{N - 2}^{″} {Δ}_{N - 2}}{6} + \frac{{y}_{N - 1}^{″} {Δ}_{N - 2}}{3} = \frac{{y}_{N} - {y}_{N - 1}}{{Δ}_{N - 1}} - \frac{{y}_{N - 1}^{″} {Δ}_{N - 1}}{3}\end{matrix} . \qquad ( 4 )$$

The set of Equations (4) can be rewritten as the set of Equations (5) as follows:

$$\begin{matrix}\frac{{Δ}_{0} + {Δ}_{1}}{3} {y}_{1}^{″} + \frac{{Δ}_{1}}{6} {y}_{2}^{″} = \frac{{y}_{0}}{{Δ}_{0}} - [ \frac{1}{{Δ}_{0}} + \frac{1}{{Δ}_{1}} ] {y}_{1} + \frac{{y}_{2}}{{Δ}_{1}} \\ \frac{{Δ}_{1}}{6} {y}_{1}^{″} + \frac{{Δ}_{1} + {Δ}_{2}}{3} {y}_{2}^{″} + \frac{{Δ}_{2}}{6} {y}_{3}^{″} = \frac{{y}_{1}}{{Δ}_{1}} - [ \frac{1}{{Δ}_{1}} + \frac{1}{{Δ}_{2}} ] {y}_{2} + \frac{{y}_{3}}{Δ - 2} \\ \frac{{Δ}_{2}}{6} {y}_{2}^{″} + \frac{{Δ}_{2} + {Δ}_{3}}{3} {y}_{3}^{″} + \frac{{Δ}_{3}}{6} {y}_{4}^{″} = \frac{{y}_{2}}{{Δ}_{2}} - [ \frac{1}{{Δ}_{2}} + \frac{1}{{Δ}_{3}} ] {y}_{3} + \frac{{y}_{4}}{{Δ}_{3}} \\ ⋮ \\ \frac{{Δ}_{N - 4}}{6} {y}_{N - 3}^{″} + \frac{{Δ}_{N - 2} + {Δ}_{N - 2}}{3} {y}_{N - 2}^{″} + \frac{{Δ}_{N - 2}}{6} {y}_{N - 1}^{″} = \frac{{y}_{N - 3}}{{Δ}_{N - 3}} - [ \frac{1}{{Δ}_{N - 3}} + \frac{1}{{Δ}_{N - 2}} ] {y}_{N - 2} + \frac{{y}_{N - 4}}{{Δ}_{N - 2}} \\ \frac{{Δ}_{N - 1}}{6} {y}_{N - 2}^{″} + \frac{{Δ}_{N - 2} + {Δ}_{N - 1}}{3} {y}_{N - 1}^{″} = \frac{{y}_{N - 2}}{{Δ}_{N - 2}} - [ \frac{1}{{Δ}_{N - 2}} + \frac{1}{{Δ}_{N - 4}} ] {y}_{N - 1} + \frac{{y}_{N}}{{Δ}_{N - 1}}\end{matrix} . \qquad ( 5 )$$

In turn, this becomes the matrix Equation (6):

$$[ \begin{matrix}\frac{{Δ}_{0} + {Δ}_{1}}{3} & \frac{{Δ}_{1}}{6} & 0 & & 0 & 0 & 0 \\ \frac{{Δ}_{1}}{6} & \frac{{Δ}_{1} + {Δ}_{1}}{3} & \frac{{Δ}_{1}}{6} & … & 0 & 0 & 0 \\ 0 & \frac{{Δ}_{1}}{6} & \frac{{Δ}_{2} + {Δ}_{3}}{3} & & 0 & 0 & 0 \\ & ⋮ & & ⋱ & & ⋮ & \\ 0 & 0 & 0 & & \frac{{Δ}_{N - 4} + {Δ}_{N - 3}}{3} & \frac{{Δ}_{N - 3}}{6} & 0 \\ 0 & 0 & 0 & … & \frac{{Δ}_{N - 4}}{6} & \frac{{Δ}_{N - 3} + {Δ}_{N - 2}}{3} & \frac{{Δ}_{N - 2}}{6} \\ 0 & 0 & 0 & & 0 & \frac{{Δ}_{N - 3}}{6} & \frac{{Δ}_{N - 1} + {Δ}_{N - 4}}{3}\end{matrix} ] \{ \begin{matrix}{y}_{1}^{″} \\ {y}_{2}^{″} \\ {y}_{3}^{″} \\ ⋮ \\ {y}_{N - 3}^{″} \\ {y}_{N - 2}^{″} \\ {y}_{N - 1}^{″}\end{matrix} \} = [ \text{} \begin{matrix}\frac{1}{{Δ}_{0}} & - \frac{1}{{Δ}_{0}} - \frac{1}{{Δ}_{2}} & \frac{1}{{Δ}_{1}} & & 0 & 0 & 0 \\ 0 & \frac{1}{{Δ}_{1}} & - \frac{1}{{Δ}_{1}} - \frac{1}{{Δ}_{1}} & … & 0 & 0 & 0 \\ 0 & 0 & \frac{1}{{Δ}_{2}} & & 0 & 0 & 0 \\ & ⋮ & & ⋱ & & ⋮ & \\ 0 & 0 & 0 & & \frac{1}{{Δ}_{N - 2}} & 0 & 0 \\ 0 & 0 & 0 & … & - \frac{1}{{Δ}_{N - 3}} - \frac{1}{{Δ}_{N - 2}} & \frac{1}{{Δ}_{N - 3}} & 0 \\ 0 & 0 & 0 & & \frac{1}{{Δ}_{N - 3}} & - \frac{1}{{Δ}_{N - 2}} - \frac{1}{{Δ}_{N - 1}} & \frac{1}{{Δ}_{N - 3}}\end{matrix} ] \{ \begin{matrix}{y}_{0} \\ {y}_{1} \\ {y}_{2} \\ {y}_{3} \\ ⋮ \\ {y}_{N - 3} \\ {y}_{N - 2} \\ {y}_{N - 1} \\ {y}_{N}\end{matrix} \} \qquad ( 6 )$$

The matrix Equation (6) may be rewritten as the set of Equations (7) as follows:

$${Fy}_{dd} = Gy \qquad ( 7 )$$

$${y}_{dd} = {F}^{- 1} Gy = Hy .$$

Choosing the values in the vector y using a least squares criterion on the collected data is shown in Equation (8) as follows:

$$E = \text{} \sum {[ {ψ}_{k} - {A}_{{i}_{k}} ( {ξ}_{k} ) {y}_{{i}_{k}} - {B}_{{i}_{k}} ( {ξ}_{k} ) {y}_{{i}_{k} + 1} - {C}_{{i}_{k}} ( {ξ}_{k} ) {y}_{{i}_{k}}^{″} - {D}_{{i}_{k}} ( {ξ}_{k} ) {y}_{{i}_{k}}^{″} ]}^{2} . \qquad ( 8 )$$

Equation (8) is the minimum deviation between the data and the spline, i.e., Equation (8) is an error function. The y values are chosen to minimize the error as defined in Equation (8). The vector of predicted values can be written as illustrated in Equation (9) as follows:

$$\begin{matrix}\hat{y} = ( {A}_{\{ k \}} + {B}_{\{ k \}} ) y + ( {C}_{\{ k \}} + {D}_{\{ k \}} ) {y}_{dd} \\ = ( {A}_{\{ k \}} + {B}_{\{ k \}} ) y + ( {C}_{\{ k \}} + {D}_{\{ k \}} ) Hy \\ = [ {A}_{\{ k \}} + {B}_{\{ k \}} + {C}_{\{ k \}} H + {D}_{\{ k \}} H ] y \\ = Ay\end{matrix} . \qquad ( 9 )$$

The elements of the matrix in brackets of Equation (9) depend upon the x-value corresponding to each data point (but this is a fixed matrix). Thus, the final equation can be determined using the pseudo-inverse. In turn, the pseudo-inverse only depends upon the x-locations of the data set and the locations where the breaks in the cubic spline are set. The implication of this is that once the geometry of the spline and the size of the image are selected, the best choice for y given a set of measured values ym is illustrated in Equation (10) as follows:

$$y = {( {A}^{T} A )}^{- 1} A \cdot {y}_{m} . \qquad ( 10 )$$

The cubic spline through the sum intensity-squared function of the image will then be given by Equation (11) as follows:

$${y}_{es} = A \cdot y . \qquad ( 11 )$$

Because the maximum values of the cubic spline are of interest, the derivative of the cubic spline is determined and utilized to determine the maximum values of the cubic spline. The cubic spline derivative is given by Equation (12) as follows:

$${𝒳}^{′} ( {x}_{k} ) = {A}_{{i}_{k}}^{′} ( {x}_{k} ) {y}_{{i}_{k}} + {B}_{{i}_{k}}^{′} ( {x}_{k} ) {y}_{{i}_{k} + 1} + {C}_{{i}_{k}}^{′} ( {x}_{k} ) {y}_{{i}_{k}}^{″} + {D}_{{i}_{k}}^{′} ( {x}_{k} ) {y}_{{i}_{k} - 1}^{″} = \text{} - \frac{{y}_{{i}_{k}}}{{Δ}_{{i}_{k}}} + \frac{{y}_{{i}_{k} + 1}}{{Δ}_{{i}_{k}}} - \frac{{Δ}_{{i}_{k}} {y}_{{i}_{k}}^{″}}{6} ( 3 {A}_{{i}_{k}}^{2} ( {x}_{k} ) - 1 ) + \frac{{Δ}_{{i}_{k}} {y}_{{i}_{k} + 1}}{6} ( 3 {B}_{{i}_{k}}^{2} ( {x}_{k} ) - 1 ) . \qquad ( 12 )$$

Equation (12) can be written as Equation (13) as follows:

$$\begin{matrix}{y}_{es}^{′} = ( {A}_{\{ k \}}^{′} + {B}_{\{ k \}}^{′} ) y + ( {C}_{\{ k \}}^{′} + {D}_{\{ k \}}^{′} ) {y}_{dd} \\ = [ {A}_{\{ k \}}^{′} + {B}_{\{ k \}}^{′} + {C}_{\{ k \}}^{′} H + {D}_{\{ k \}}^{′} H ] y \\ = {A}^{′} y\end{matrix} . \qquad ( 13 )$$

Once the current values of y are found, the cubic spline, ycs, and its derivative, y′cs, can be calculated. The cubic spline data may include “blocks” of data that includes values above a predetermined threshold. A pipe block is formed by the liquid flowing out of the tube into the drip chamber 59 and a pool block is formed as the liquid collects at the gravity end of the drip chamber 59 (see FIG. 8).

Equation Chapter (Next) Section 1 The following algorithm may be applied to the cubic spline data: (1) determine the local maxima of the cubic spline data using the derivative information; (2) determine the block surrounding each local maxima by including all points where the cubic spline value is above a threshold value; (3) merge all blocks which intersect; (4) calculate information about the block of data including the center of mass (intensity), the second moment of the mass (intensity), the lower x-value of the block, the upper x-value of the block, the mean value of the original sum of intensity squared data in the block, the standard deviation of the original sum of intensity squared data in the block, and the mean intensity of a high-pass filtered image set in the block; and (5) interpret the collected data to obtain information about when drops occur and when the system is streaming.

The mean intensity of a high-pass filtered image set in the block is used to determine if the block created by each contiguous range of spline data is a result of a high frequency artifact (e.g., a drop) or a low frequency artifact. This will act as a second background filter which tends to remove artifacts such as condensation from the image. That is, all previous images in an image memory buffer (e.g., 30 previous frames, for example) are used to determine if the data is a result of high frequency movement between frames. If the block is a result of low frequency changes, the block is removed, or if it is a result of high frequency changes, the block is kept for further analysis. A finite impulse response filter or an infinite impulse response filter may be used.

Each block is plotted over its physical extent with the height equal to the mean value of the data within the block. Ifa block has a mean value of the high-pass filtered image less than the threshold, it is an indication that it has been around for several images and thus may be removed.

Free flow conditions may be determined by the processor 90 (see FIG. 6 or 8) to exist using the blocks when the pipe block extends nearly to the pool block, the pipe block and the pool block merge together, and/or the summed range of widths of the pool and pipe blocks (or all blocks) is greater than a predetermined threshold, e.g., the total extent of the blocks exceeds 380 pixels in width. The processor 90 may detect a drop when the transition of the pipe block from a larger width to a shorter width occurs as a result of a drop formation in the tube and as the drop leaves the pipe (i.e., tube) opening of the drip chamber 59. The processor 90 may detect this by looking at the ratio of the current pipe block width to the previous image's pipe block width, e.g., an image where the ratio is less than 0.9 as is also a local minima may be considered by the processor 90 to be an image formed immediately after a drop has formed.

Various filtering algorithms may be used to detect condensation or other low frequency artifacts, such as: if a block has a low mean value in the high-pass filtered image, then it may be condensation. This artifact can be removed from consideration. Additionally or alternatively, long blocks (e.g., greater than a predetermined threshold) with a low high-pass mean value are possibly streams because stream images tend to remain unchanging; the processor 90 may determine that long blocks greater than a predetermined threshold corresponds to a streaming condition. Additionally or alternatively, an algorithm may be used on the current image to detect free flow conditions.

The processor 90 may, in some specific embodiments, use the block data to count the drops to use the system 84 as a drop counter. The processor 90 may also use width changes in the pool block as a drop disturbs the water to determine if a bubble formed when the drop hits the pool. For example, the processor 90 may determine that blocks that form below the pool block are from bubbles that formed when the drop hit the water. The bubble may be filtered out by the processor 90 when determining if a predetermined value of total block ranges indicates that a free flow condition exists.

In some embodiments of the present disclosure, the depth of field of the system 84 may have a narrow depth of field to make the system 84 less sensitive to condensation and droplets on the chamber walls. In some embodiments, a near focus system may be used.

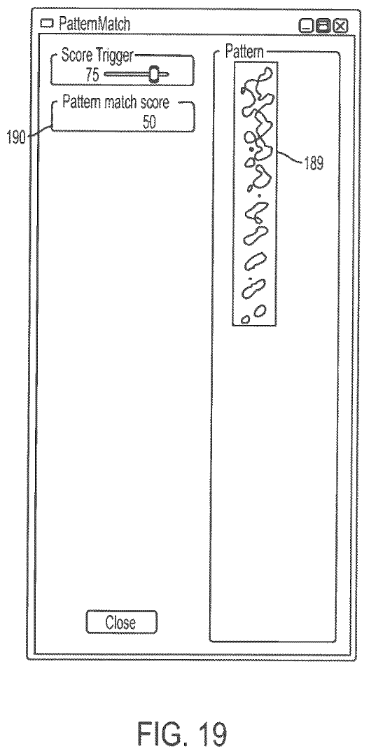

Referring now to FIG. 19, in another embodiment of the present disclosure, a template 189 is used to determine if a free flow condition exists. The template 189 is used by the processor 90 of FIG. 8 to determine a pattern match score 190 when performing a template match algorithm on an image, e.g., the image 94 of FIG. 13. For example, the template 189 may be compared to the image 94 to determine if a portion or all of the image 94 closely matches the template 189. As previously mentioned, the image 94 of FIG. 13 is a difference between a background image and an image captured by the image sensor 63 of FIG. 8 that has each pixel converted to either a black pixel if the difference value for that pixel is below a threshold value or a white pixel if the difference value for that pixel is above a threshold value. All pixels of the image 94 will be either a white pixel or a black pixel. If the pattern match score 190 is above a predetermined threshold, a free flow condition is determined to exist. The template matching method may utilize a template matching algorithm as found in the Open Source Computer Vision (“OpenCV”) library. For example, the template 189 may be used with the matchTemplate( ) function call of the OpenCV library using the CV_TM_CCOEFF method or the method of CV_TM_CCOEFF_NORMED. The CV_TM_CCOEFF method uses the pattern matching algorithm illustrated in Equation (14) as follows:

$$ℛ ( 𝓍 , 𝓎 ) = {\sum }_{{𝓍}^{′} , {𝓎}^{′}} ( {𝒯}^{′} ( {𝓍}^{′} , {𝓎}^{′} ) \cdot {I}^{′} ( 𝓍 + {𝓍}^{′} , 𝓎 + {𝓎}^{′} ) ) , \qquad ( 14 ) ,$$

$$where :$$

$${𝒯}^{′} ( {𝓍}^{′} {𝓎}^{′} ) = 𝒯 ( {𝓍}^{′} , {𝓎}^{′} ) - 1 / ( 𝓌 \cdot 𝒽 ) \cdot {\sum }_{{𝓍}^{″} \cdot {𝓎}^{″}} 𝒯 ( {𝓍}^{″} , {𝓎}^{″} )$$

$${I}^{′} ( 𝓍 + {𝓍}^{′} , 𝓎 + {𝓎}^{′} ) = I ( 𝓍 + {𝓍}^{′} , 𝓎 + {𝓎}^{′} ) - 1 / ( 𝓌 \cdot 𝒽 ) \cdot {\sum }_{{𝓍}^{″} , {𝓎}^{″}} I ( 𝓍 + {𝓍}^{″} , 𝓎 + {𝓎}^{″} ) ; The I$$

denotes the image, the T denotes the template, and the R denotes the results. The summation is done over the template and/or the image patch, such that: x′=0 . . . w−1 and y′=0 . . . h−1.

The results R can be used to determine how much the template T is matched at a particular location within the image I as determined by the algorithm. The OpenCV template match method of CV_TM_CCOEFF_NORMED uses the pattern matching algorithm illustrated in Equation (15) as follows:

$$ℛ ( 𝓍 , 𝓎 ) = \frac{{\sum }_{{𝓍}^{′} , {𝓎}^{′}} ( {𝒯}^{′} ( {𝓍}^{′} , {𝓎}^{′} ) \cdot {I}^{′} ( 𝓍 + {𝓍}^{′} , 𝓎 + {𝓎}^{′} ) )}{\sqrt{{\sum }_{{𝓍}^{′} , {𝓎}^{′}} {{𝒯}^{′} ( {𝓍}^{′} , {𝓎}^{′} )}^{2} \cdot {\sum }_{{𝓍}^{′} , {𝓎}^{′}} {{I}^{′} ( 𝓍 + {𝓍}^{′} , 𝓎 + {𝓎}^{′} )}^{2}}} . \qquad ( 16 )$$

In another embodiment of the present disclosure, the template matching algorithm uses a Fast Fourier Transform (“FFT”). In some embodiments, any of the methods of the matchTemplate( ) function of OpenCV may be used, e.g., CV_TM_SQDIFF, CV_TM_SQDIFF_NORMED, CV_TM_CCORR, and/or CV_TM_CCORR_NORMED.

The CV_TM_SQDIFF uses the pattern matching algorithm illustrated in Equation (17) as follows:

$$R ( x , y ) = {\sum }_{{x}^{′} , {y}^{′}} {( T ( {x}^{′} , {y}^{′} ) - I ( x + {x}^{′} , y + {y}^{′} ) )}^{2} . \qquad ( 17 )$$

CV_TM_SQDIFF_NORMED uses the pattern matching algorithm illustrated in Equation (18) as follows:

$$R ( x , y ) = \frac{{\sum }_{{x}^{′} , {y}^{′}} {( T ( {x}^{′} , {y}^{′} ) - I ( x + {x}^{′} , y + {y}^{′} ) )}^{2}}{\sqrt{{\sum }_{{x}^{′} , {y}^{′}} {T ( {x}^{′} , {y}^{′} )}^{2} \cdot {\sum }_{{x}^{′} , {y}^{′}} {I ( x + {x}^{′} , y + {y}^{′} )}^{2}}} . \qquad ( 18 )$$

CV_TM_CCORR uses the pattern matching algorithm illustrated in Equation (19) as follows:

$$R ( x , y ) = {\sum }_{{x}^{′} , {y}^{′}} ( T ( {x}^{′} , {y}^{′} ) - I ( x + {x}^{′} , y + {y}^{′} ) ) . \qquad ( 19 )$$

CV_TM_CCORR_NORMED uses the pattern matching algorithm illustrated in Equation (20) as follows:

$$R ( x , y ) = \frac{{\sum }_{{x}^{′} , {y}^{′}} ( T ( {x}^{′} , {y}^{′} ) \cdot {I}^{′} ( x + {x}^{′} , y + {y}^{′} ) )}{\sqrt{{\sum }_{{x}^{′} , {y}^{′}} {T ( {x}^{′} , {y}^{′} )}^{2} \cdot {\sum }_{{x}^{′} , {y}^{′}} {I ( x + {x}^{′} , y + {y}^{′} )}^{2}}} . \qquad ( 20 )$$

In yet another embodiment of the present disclosure, a template of a grayscale image of a free flow condition is compared to an image taken by the image sensor 63 of FIG. 8 to determine if a free flow condition exists. In some embodiments, the template matching function within the OpenCV library may be utilized.

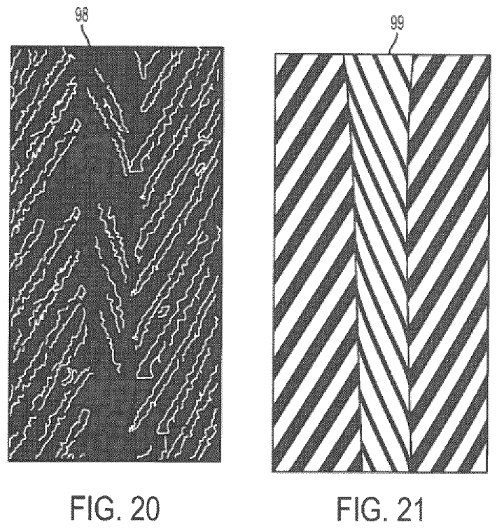

Refer now to FIGS. 20 and 21; in yet an additional embodiment of the present disclosure, the algorithm to determine when a free flow condition exists, e.g., as executed by the processor 90 of FIG. 8, may utilize an algorithm to determine if a template pattern matches an array of pixels utilizing edge detection followed by line detection. As shown in FIG. 20, an image 98 is formed from an image 99 of FIG. 21, by using edge detected followed by line detection. The resulting lines may be utilized by the processor 90 to determine that a free flow condition exists. As shown in FIG. 20, the feature which shows up after this processing by the processor 90 are lines that have a different slope than the expected 45° slope of the background reference image. The lines having the angle of the background image may be filtered out of FIG. 20, in some embodiments. The lines may be detected as edges using a Canny algorithm as found in the OpenCV library. The Hough algorithm also found in the OpenCV library may be used to determine the slope of the lines.

One type of Hough transfer uses an algorithm described in Progressive Probabilistic Hough Transform by J. Matas, C. Galambos, and J. Kittler in 1998 (“Algorithm 1”). However, the following “Alternative Hough” transform may be utilized and is shown in pseudo code form in Table 1 (“Algorithm 2”). Algorithm 2 selects two pixels at random and calculates the Hough transform of the line passing through these two points. Algorithm 2 is shown in Table 1 as follows:

TABLE 1

```
Alternative Hough Transform Pseudocode
  If the image is empty, then exit.
  Randomly select two pixels and update the accumulator
  Required Operations
  Two random numbers
  One inverse tangent
  Check if the new location is higher than the threshold I. If not, goto
  Operations
  One logical operation
  Look along a corridor specified by the peak in the accumulator, and find
the longest segment of pixels either continuous or exhibiting a gap not exceeding a
given threshold.
  Remove the pixels in the segment from the input image.
  Unvote from the accumulator all the pixels from the line that have
previously voted.
  If the line segment is longer than the minimum length add it to the
output list
  Goto 1.
```

If the line comprises a proportion, p, of the total points, then the likelihood that we will see a result in the representative (r,θ)-bin is p for Algorithm 1 and p2 for Algorithm 2. Generally, in some embodiments, a proportion test has at least 5 positive results and 5 negative results. Assuming that it is more likely to see negative results than positive results, in some embodiments, the Algorithms 1 and 2 continue to search for lines until there are at least 5 positive results in a particular bin.

The probability of seeing a fifth positive result in Algorithm 1 after N≥5 tests is shown in Equation (21) as follows:

$${p}_{1} ( 5 on N ) = p ( 4 in N - 1 ) \cdot p = \frac{( N - 1 ) !}{4 ! ( N - 5 ) !} {{p}^{5} ( 1 - p )}^{N - 5} , \qquad ( 21 )$$

and the probability in Algorithm 2 is shown in Equation (22) as follows:

$${p}_{2} ( 5 on N ) = p ( 4 in N - 1 ) \cdot {p}^{2} = \frac{( N - 1 ) !}{4 ! ( N - 5 ) !} {{p}^{10} ( 1 - {p}^{2} )}^{N - 5} . \qquad ( 22 )$$

Table 2, shown below, shows the number of tries to have a 50% chance of seeing 5 successes, p1,50 and p2,50, as well as the number of tries to have a 90% chance of seeing 5 successes, p1,90 and p2,90.

TABLE 2

| | 1.50 | 1.90 | 2.50 | 2.90 | 50 | 90 |
|---|---|---|---|---|---|---|
| .5 | | 4 | 0 | 1 | .22 | .21 |
| .25 | 9 | 0 | 6 | 27 | | .23 |
| .125 | 9 | 2 | 99 | 11 | .67 | .24 |
| .0625 | 6 | 27 | 197 | 046 | 5.75 | 6.11 |

Table 2 shows that the increase in the number of tries between Algorithm 1 and Algorithm 2 to see 5 positive results is approximately 1/p. There should be 1 positive result in 1/p trials when the proportion is p.

Algorithm 2's computationally expensive operation is, in some embodiments, the arc tangent function, which may be about 40 floating point CPU operations. There are approximately 2N floating point operations in Algorithm 1's equivalent step. The Hough transform of a 640×480 pixel image with full resolution has N equal to 2520, while the Hough transform of a 1080×1920 pixel image has N equal to 7020. This implies that Algorithm 2 has a speed advantage over Algorithm 1 when p is greater than 0.008 for a 640×480 image and when p is greater than 0.003 for a 1080×1920 image.

In some embodiments, it is assumed that every bin in the Hough transform space is equally likely to be occupied in the presence of noise. This simplification speeds up the thresholding decision; however, in some embodiments, this assumption is not true. The primary effect of the simplification is to underestimate the probability that is seen in values greater than one in the Hough transform with a corresponding likelihood of falsely declaring that a line exists. For a particular combination of image size and Hough transform bin arrangement, the true probabilities can be pre-computed. This allows the false alarm rate to be minimized without a corresponding increase in computation. With additional restrictions on the type of imagery, even more accurate estimates of the probability of seeing a value in a bin of the Hough transform is possible.

There are additional forms of the Hough transform which parameterizes different features. For example, there is a three-element parameterization of circles, (x,y,r), where x and y specify the center and r is the radius. Algorithm 2 can work using these parameterizations as well. For the circle example, Algorithm 2 would select three pixels at random and calculate the circle passing through them.

Algorithm 2 would have a similar speed advantage for features comprising a suitably large portion of the total pixels considered. It would also have a significant advantage in storage required, since the Hough transform could be stored in a sparse matrix, while the Algorithm 1's analog would require a full-size matrix.

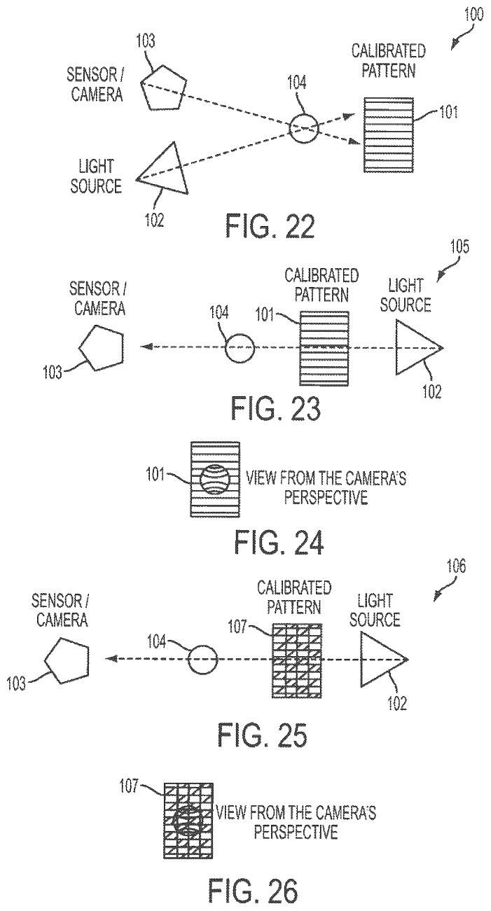

Referring now to FIGS. 22-26, which illustrate various background patterns that may be used to detect a free flow condition or estimate the size of a drop of liquid. The image sensor 103 may be used with the background patterns of FIGS. 22-26 and may be the image sensor 11 of FIG. 1, the image sensor 68 of FIG. 5, the image sensor 63 of FIG. 6, or the image sensor 63 of FIG. 8, each of which may be coupled to a respective processor for processing the images from the image sensor, such as the processor 15 of FIG. 1 or the processor 90 of FIG. 8.

FIG. 22 is a block diagram of an imaging system 100 for use with the drip chamber 104 (e.g., a drip chamber 4 of FIG. 1) having a background pattern 101 with stripes and a light source 102 shining on the stripes from an adjacent location to an image sensor 103 in accordance with an embodiment of the present disclosure. Any drops or free flow streams within the drip chamber 104 distorts the image taken by the image sensor 103. A processor coupled to the image sensor 103 (e.g., processor 15 of FIG. 1) can use the distortions of the background pattern 101 as captured by the image sensor 103 to estimate a flow rate and/or detect free flow conditions.

FIG. 23 is a block diagram of an imaging system 105 for use with the drip chamber 104 having a background pattern 101 with stripes and a light source 102 shining on the stripes from behind the background pattern 101 relative to an opposite end to an image sensor 103 in accordance with an embodiment of the present disclosure. FIG. 24 shows an image from the image sensor 103 of FIG. 23 when a drop distorts the background pattern 101 of FIG. 23 in accordance with an embodiment of the present disclosure. Note that as shown in FIG. 24, the background pattern's 101 stripes are distorted by the drop (or will be distorted by a free flow stream) in the drip chamber 104 as captured in images by the image sensor 103. This distortion may be used to estimate the drop size, to calculate the flow rate through a drip chamber, or to determine if a free flow condition exists within the drip chamber.

FIG. 25 shows a block diagram of an imaging system 106 for use with a flow meter having a background pattern 107 with a checkerboard pattern and a light source 102 shining on the stripes from behind the background pattern 107 relative to an opposite end to an image sensor 103 in accordance with an embodiment of the present disclosure. FIG. 26 shows an image from the image sensor 103 of FIG. 25 when a drop distorts the background pattern 107 of FIGS. 25-26 in accordance with an embodiment of the present disclosure. In yet another embodiment of the present disclosure, a background pattern having a plurality of random dots and/or circles may be utilized by an imaging system disclosed herein.

Referring to FIGS. 22-26, the “lensing” of a drop (i.e., the distortion of the background pattern from the view of an image sensor) may be used to measure the radius of the drop. The radius of the drop corresponds to how much and what effect the drop has on any light passing through it. By measuring the change to the calibration grid (i.e., the background pattern) as seen through the drop, the radius, and hence the volume of the drop, can be calculated. For example, the magnification of a test grid of known size as seen through the drop could be measured optically and the radius inferred from this measurement. In some embodiments of the present disclosure, the relationship between the radius and the drop may be calculated and/or may be determined using a lookup table that has been generated empirically.

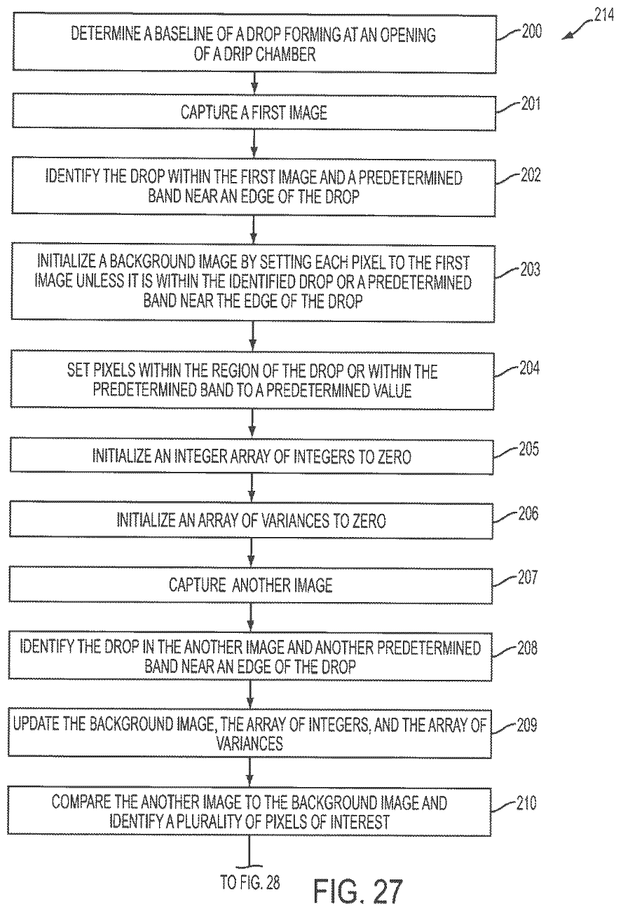

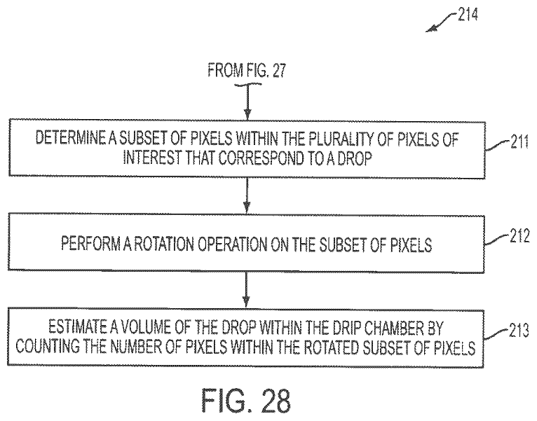

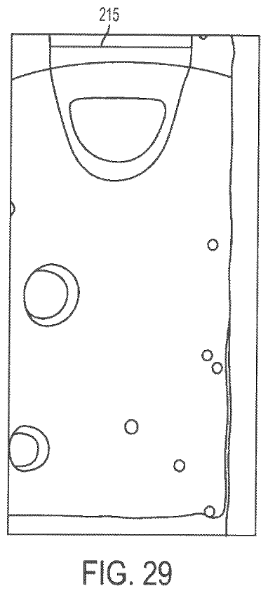

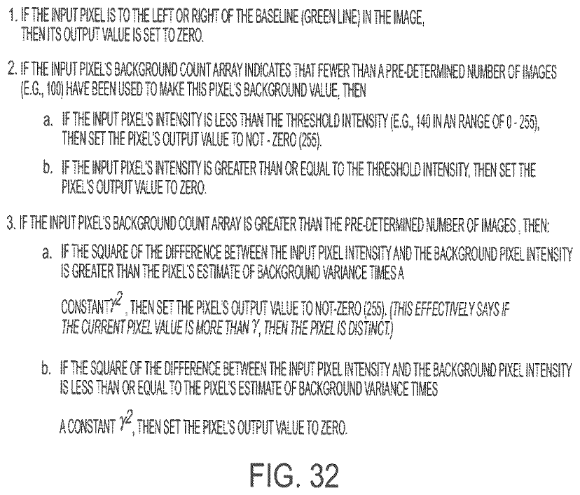

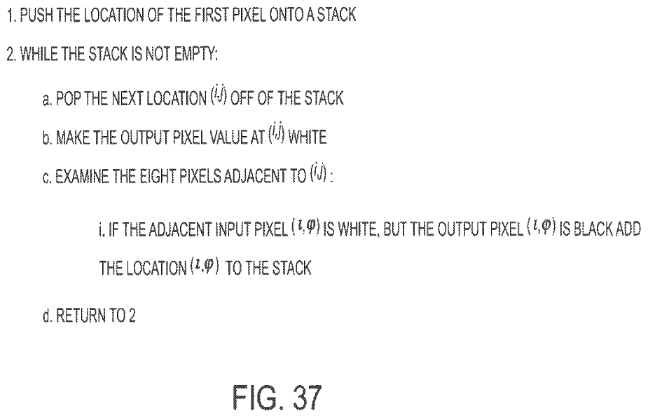

FIGS. 27-28 show a flow chart diagram illustrating a method for estimating a volume of a drop within a drip chamber in accordance with an embodiment of the present disclosure. That is, FIGS. 27-28 illustrate a method 214. Method 214 will be also described with reference to FIGS. 29-37. FIGS. 29-31 and 33-36 illustrate images used or generated by a flow meter to estimate a volume of a drop within a drip chamber in accordance with an embodiment of the present disclosure. FIGS. 32 and 37 illustrate pseudo code that may be used by the method 214 of FIGS. 27-28.

The method 214 of FIGS. 27 and 28 may be implemented by the flow meter 7 of FIG. 1, the flow meter 67 of FIG. 5, the imaging system 78 of FIG. 6, the imaging system 84 of FIG. 8, or other flow meter of an imaging system disclosed herein (each with or without a background pattern and/or with or without active illumination).

The method 214 includes acts 200-213. Act 200 determines a baseline of a drop forming at an opening of a drip chamber. Act 201 captures a first image. The first image may be captured using a uniform backlight. In some embodiments, the first image may be captured using a background pattern and/or an exposure algorithm as described herein. Acts 200 and 201 may be performed simultaneously. FIG. 29 shows an image with the baseline 215 overlaid. The baseline 215 may be a predetermined group of pixels or may be generated using fiducial markers disposed on the opening of the drip chamber and/or on a background pattern (not shown in FIG. 29). The first image is used by the method 214 to initialize a background image, μi,j, a variance array, si,j, and an integer array, Ii,j. The background image may have i by j pixels, while the variance array and the integer array may be 2-D arrays that also have a size of i by j.

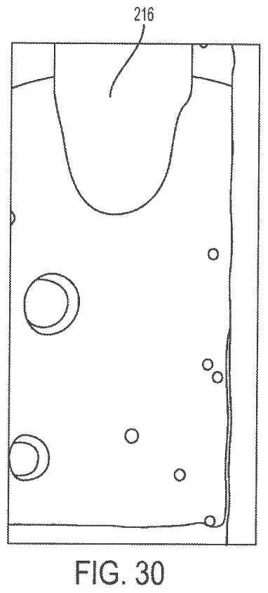

Act 202 identifies the drop within the first image and a predetermined band near an edge of the drop (e.g., the band may be a predetermined number of pixels beyond the edge of the drop). Act 203 initializes a background image by setting each pixel to the same value as the first image (for that respective location) unless it is within the identified drop or a predetermined band near the edge of the drop. Act 204 sets pixels within the region of the drop or within the predetermined band to a predetermined value. FIG. 30 shows an example background image created after initialization. In the exemplary image of FIG. 30, the area of the drop and of a band beyond the edge of the drop, designated generally as 216, is set to a predetermined value, e.g., 140.

For example, when the method creates the first background image, every pixel in the background image that is part of the drop or a band outside of an edge of the drop is set to a default threshold value, e.g. 140 out of an intensity range of 0-255.

Act 205 initializes the integers of the array of integers to zeros. Act 206 initializes the values within the array of variances to zeros. The integer array is the same size as the image. The integer array counts how often each pixel of the background image has been updated with new information and is initialized to all zeros. The array of variances (e.g., an array of the data type “double”) is also the same size as the background image and contains an estimate of the variance of the intensity of each pixel within the background image.

Act 207 captures another image, and act 208 identifies the drop in the another image and another predetermined band near an edge of the drop. Act 209 updates the background image, the array of integers, and the array of variances.

As additional images are captured, the background image may be updated. For example, when an image is collected by the system, the background algorithm evaluates every pixel. If a pixel is considered part of the drop or its guard band, then its value in the background image is not altered.

If a pixel is not considered part of the drop or its guard band: (1) if the pixel's corresponding integer in the integer array is zero, the pixel's value in the background image is set equal to the pixel's value in the input image; or (2) if the pixel's count is greater than 0, then the background image value for that pixel is updated using a low pass filter. In some embodiments, any style of filter may be used, such as a high pass filter, a bandpass filter, etc. One low pass filter that may be used is illustrated in Equation (23) as follows:

$${P}_{background , i , j} = {P}_{background , i , j} ( 1 - {α}_{background} ) + {α}_{background} {P}_{input , i , j} . \qquad ( 23 )$$

In addition, the variance array may be updated using Equations (24) as follows:

$$\begin{matrix}{σ}_{temp}^{2} = {( {P}_{background , i , j} - {P}_{input , i , j} )}^{2} \\ {σ}_{background , i , j}^{2} = {σ}_{background , i , j}^{2} ( 1 - {β}_{background} ) + {β}_{background} {σ}_{temp}^{2}\end{matrix} . \qquad ( 24 )$$

Note that the filter used for both operations is an exponential filter; however, in additional embodiments, other suitable filters may be used, such as other low-pass filters. The variance estimate can be performed in any known way or using a stand in for the estimate, e.g., using standard deviation.

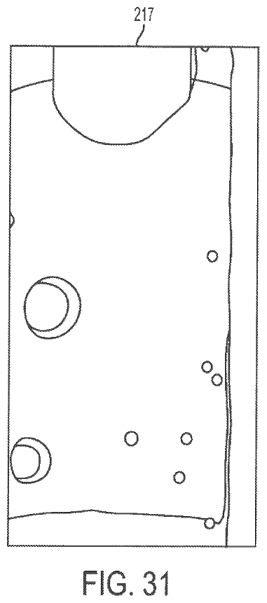

The new estimates of each pixel's background intensity (mean value), the number of images used to update each pixel's mean and variance, and each pixel's variance (e.g., an approximation to the true variance and/or a value that is proportional to the variance) are used to update the arrays. That is, each additional image captured may be used to update the background image, the array of integers, and the array of variances. After several images have been processed, the background image may appear as FIG. 31. Note that this image still has a region (the uniformly medium gray area, designated generally as 217) where the pixels have never changed from the initial threshold value. This region has been considered part of the drop or its guard band in every image.

Act 210 compares the another image (e.g., current or most recent image) to the background image and identifies a plurality of pixels of interest. Act 211 determines a subset of pixels within the plurality of pixels of interest that corresponds to a drop.

The comparison of act 210 compares the another image pixel-by-pixel to the background image. Out of this comparison comes an array the same size as the image where every pixel has a value of zero or not zero (255).

Act 210 may be implemented by the pseudo code shown in FIG. 32. That is, the determination of this threshold value is made in accordance with the following: If the input pixel is to the left or right of the baseline in the image, then its output value is set to zero (Line 1); if the input pixel's background count array indicates that fewer than a pre-determined number of images (e.g., 100) have been used to make this pixel's background value (Line 2), then: if the input pixel's intensity is less than the threshold intensity (e.g., 140 in a range of 0-255), then set the pixel's output value to not-zero (255) (Line 2a); or if the input pixel's intensity is greater than or equal to the threshold intensity, then set the pixel's output value to zero (Line 2b); and if the input pixel's background count array is greater than the pre-determined number of images (Line 3), then: if the square of the difference between the input pixel intensity and the background pixel intensity is greater than the pixel's estimate of background variance times a constant γ², then set the pixel's output value to not-zero (255) (Line 3a) (that is, if the difference between current pixel value and the background image is more than γ, then the pixel is distinct); or if the square of the difference between the input pixel intensity and the background pixel intensity is less than or equal to the pixel's estimate of background variance times a constant γ², then set the pixel's output value to zero (see Line 3b). Line 3 captures portions of the image that are altered by the presence of a drop, but which are made a higher intensity.

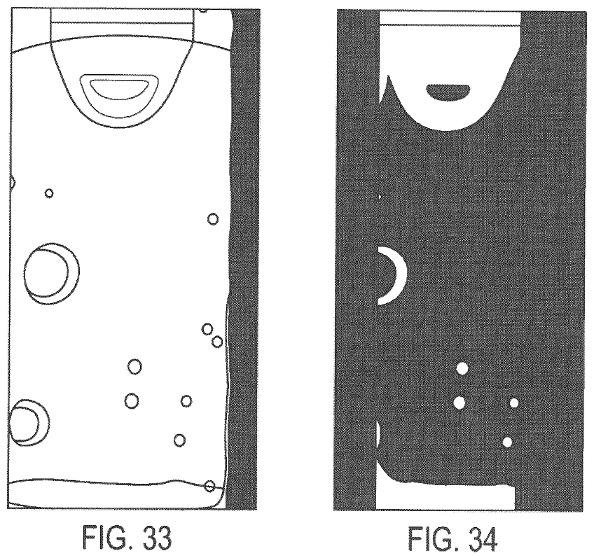

When act 210 is implemented as an algorithm, the algorithm is initialized, and the input and output of this thresholding algorithm will look like the images in FIGS. 33 and 34, respectively. Because the number of images used in estimating the background image is initially small, the only criterion applied are shown as lines (1) and (2) above because there have not been enough images used for the integer array to have a value beyond the threshold for certain respective pixels. This may result in many low-intensity regions being identified as distinct, including poorly illuminated edges and condensation on the chamber walls.

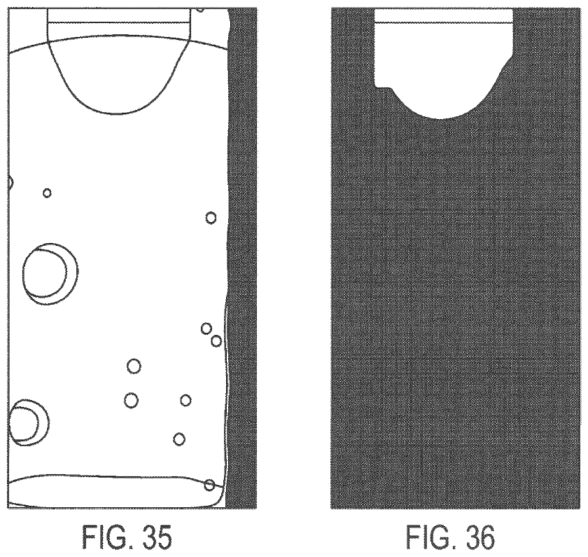

After enough images have been gathered such that most (or all) of the pixels of the background image have been generated with a sufficient number of pixels, lines (3), (3a), and (3b) of FIG. 32 are utilized. After thresholding, the background is largely black with an occasional noisy pixel exceeding the variance threshold, as shown in FIGS. 35 and 36 (which show an image captured by the camera and the results of the comparison algorithm described above, respectively).

As previously mentioned, after act 210, act 211 determines which of a subset of pixels within the plurality of pixels of interest corresponds to a drop. Act 211 may be implemented by the pseudo code shown in FIG. 37. That is, the threshold image is passed to an algorithm which finds the connected component representing the drop as illustrated by the pseudo code of FIG. 37.

The binary image after processing the pseucode of FIG. 32 is evaluated to find the binary component which occupies the space given by the drop. The algorithm is passed the location of a pixel on the baseline which is white (or it is passed the center pixel of the longest stretch of contiguous white pixels on the line).

Once the algorithm has an initial white pixel, it performs the algorithm illustrated by the pseudo code shown in FIG. 37. The pseudo code determines locations that include white pixels that have a path to the baseline (i.e., a white pixel path). Line 1 pushes the location of the first pixel onto a stack. Line 2 performs a while loop while the stack is not empty. The while loop includes lines (2a)-(2d). Line 2a pops the next location (i,j) off of the stack. Line 2b makes the output pixel value at (i,j) white. Line 2c examines the eight pixels adjacent to (i,j). Line (2ci) is an “if statement,” and if the adjacent input pixel (ι,φ) is white, but the output pixel (ι,φ) is black, line 2c adds the location (ι,φ) to the stack. Line 2d return to line 2 to continue the while loop (if the stack remains empty).

This algorithm will set to white all output-pixel locations which can be connected to the input pixel's location by a continuous path of white input pixels. The left boundary of the drop is found by stepping through each row of pixels from the left edge until the algorithm hits a white pixel. The right boundary is found by stepping from the right edge of the image until it hits a white pixel. The first row where it is possible to step from the left edge to the right edge without hitting a white pixel is where the drop is considered to end.

The pseudo code shown in FIG. 37 is a one-pass version of a connected-component labeling algorithm. However, other connected-component labeling algorithms or other suitable algorithms may be used to determine which pixels correspond to the drop.

Act 212 of FIG. 28 performs a rotation operation on the subset of pixels. Act 213 estimates a volume of the drop within the drip chamber by counting the number of pixels within the rotated subset of pixels. The total number of pixels within the 3-D version of the drop is counted; and because each pixel corresponds to a distance, the number of pixels may be used to estimate the volume of the drop.

### Imaging System Optics

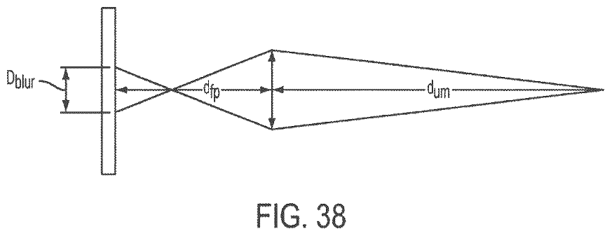

FIGS. 38-42 facilitate the following description of the optics of an imaging system disclosed herein. For example, an image sensor disclosed herein may be an image sensor cube manufactured by OmniVision of 4275 Burton Drive, Santa Clara, California 95054; and, for example, the image sensor cube may be one manufactured for phone image sensor applications. In some embodiments of the present disclosure, an image sensor disclosed herein may use a fixed focus and have a depth of field (“DOF”) from 15 centimeters to infinity.

The image sensor may have the blur circle of a point imaged in the range of the image sensor entirely contained within the area of a single pixel. The focal length of the image-sensor lens may be 1.15 millimeters, the F #may be 3.0, and the aperture of the lens of the image sensor may be 0.3833 millimeter. A first order approximation of the optical system of one or more of the image sensors may be made using matrix equations, where every ray, r, is represented as the vector described in Equation (25) as follows:

$$r = \{ \frac{h}{θ} \} . \qquad ( 25 )$$

In Equation (25) above, h is the height of the ray at the entrance to the image sensor, and θ is the angle of the ray. Referring to FIG. 38, when imaging a hypothetical point at a distance dim from the lens of one of the image sensors (which has focal length f) and the lens is a distance dfp from the focal plane, the corresponding matrix, Mcam, describing the image sensor is described by Equation (26) as follows:

$${M}_{cam} = [ \begin{matrix}1 & {d}_{fp} \\ 0 & 1\end{matrix} ] \cdot [ \begin{matrix}1 & 0 \\ - \frac{1}{f} & 1\end{matrix} ] \cdot [ \begin{matrix}1 & {d}_{im} \\ 0 & 1\end{matrix} ] . \qquad ( 26 )$$

To find the place on the focal plane, fp, where the ray strikes, a matrix multiplication as described in Equation (27) as follows may be used:

$$\{ \frac{{h}_{fp}}{{θ}_{fp}} \} = {M}_{cam} \cdot \{ \frac{{h}_{im}}{{θ}_{im}} \} . \qquad ( 27 )$$

As illustrated in FIG. 38, the diameter of the blur circle, Dblur, is shown as approximately the distance between the two points illustrated in FIG. 38. This distance is found by tracing rays from the point, dim, away from the lens on the optical axis to the edges of the lens and then to the focal plane. These rays are given by the vectors shown in (28) as follows:

$$\{ \begin{matrix}0 \\ ( \pm {tan}^{- 1} \frac{{D}_{lens}}{2 * {d}_{im}} )\end{matrix} \} . \qquad ( 28 )$$

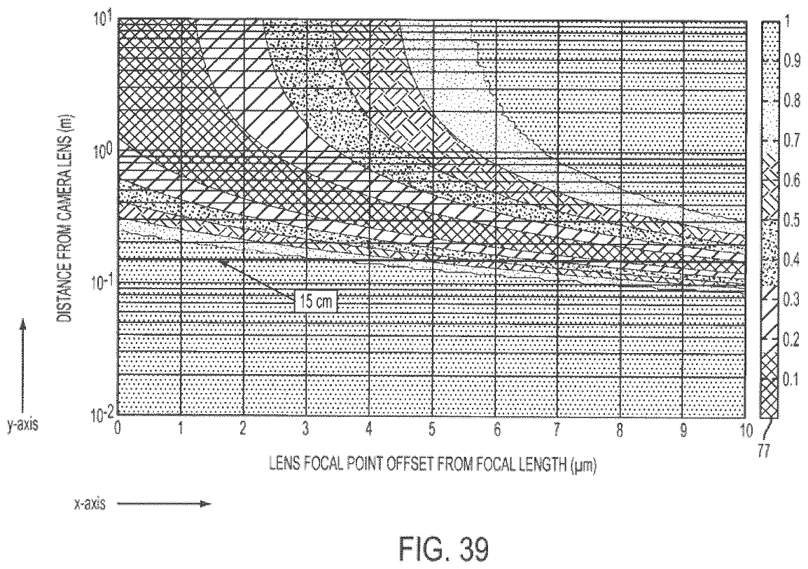

As shown in FIG. 39, the blur circle, Dblur, is calculated and shown for a variety of lens-to-focal plane separations and lens-to-image separations. A contour map 77 is also shown in FIG. 39. The x-axis shows the distance in microns between the focal plane and a point located a focal length away from the lens of an image sensor. The y-axis shows the distance in meters between the lens and the point being imaged. The values creating the contour map 77 is the blur size divided by the pixel size; therefore, anything about 1 or less is sufficient for imaging. As shown in FIG. 39, the focal plane is located a focal length and an additional 5 micrometers away from the lens.

The image sensor may utilize a second lens. For example, an image sensor may utilize a second lens to create a relatively larger depth of field and a relatively larger field of view. The depth of field utilizing two lenses can be calculated using the same analysis as above, but with the optical matrix modified to accommodate for the second lens and the additional distances, which is shown in Equation (29) as follows:

$${M}_{sys} = [ \begin{matrix}1 & {d}_{fp} \\ 0 & 1\end{matrix} ] \cdot [ \begin{matrix}1 & 0 \\ - \frac{1}{{f}_{cam}} & 1\end{matrix} ] \cdot [ \begin{matrix}1 & {d}_{lens} \\ 0 & 1\end{matrix} ] \cdot [ \begin{matrix}1 & 0 \\ - \frac{1}{{f}_{lens}} & 1\end{matrix} ] \cdot [ \begin{matrix}1 & {d}_{im} \\ 0 & 1\end{matrix} ] . \qquad ( 29 )$$

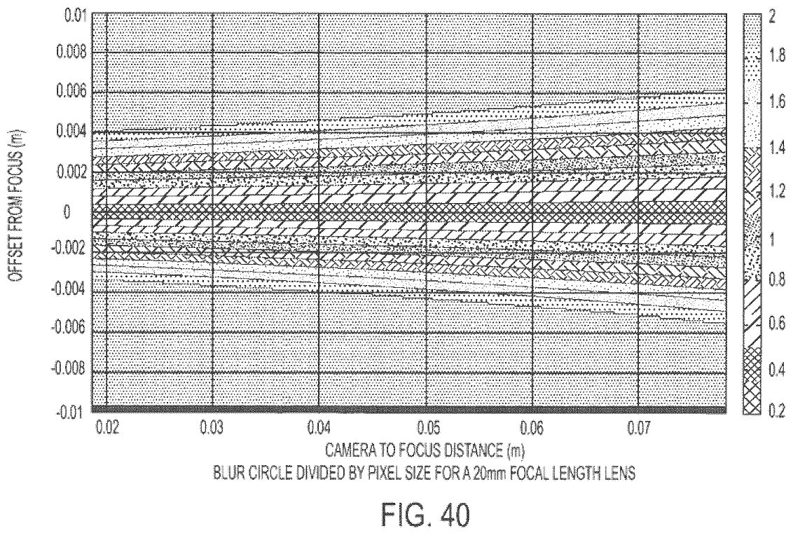

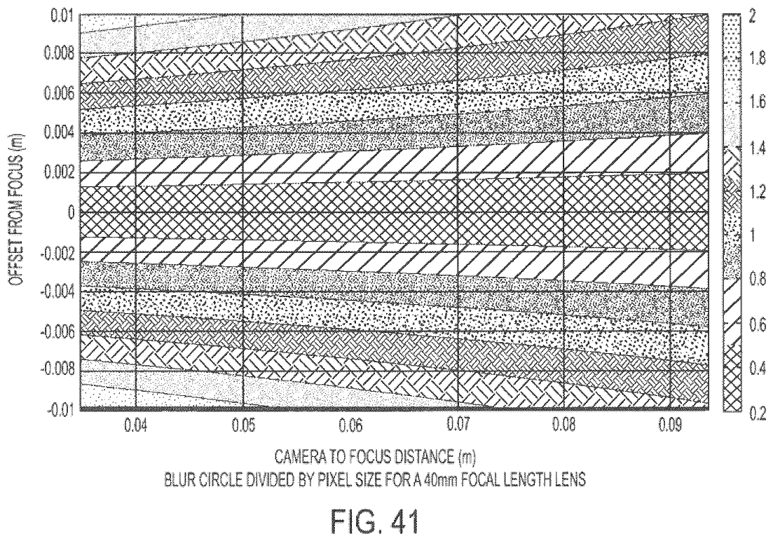

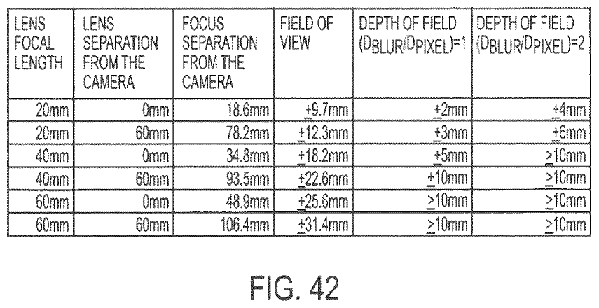

FIGS. 40 and 41 illustrate the field changes with the separation between the lens and the image sensor and the corresponding change in the focus of the image sensor. FIGS. 40 and 41 show the blur circle divided by the pixel size. FIG. 40 shows the blur circle divided by pixel size when a 20 millimeter focal-length lens is used. FIG. 41 shows the blur circle divided by pixel size when a 40 millimeter focal length lens is used. The corresponding fields of view about the optical axis for the corners of the two configurations of FIGS. 40 and 41 are shown in the table in FIG. 42.

As shown in FIG. 42, in some embodiments, the image sensor may utilize a 40 mm to 60 mm focal-length lens; this configuration may include placing an image sensor about 2 inches from the focus. In other embodiments of the present disclosure, other configurations may be used including those not shown in FIG. 42.

For example, the following analysis shows how the depth of field can be set for an image sensor using a lens of focal length, f, a distance, z, from the focal plane, and a distance, d, from a point in space; a matrix of the system is shown in Equation (30) as follows:

$$M = [ \begin{matrix}1 & z \\ 0 & 1\end{matrix} ] \cdot [ \begin{matrix}1 & 0 \\ - \frac{1}{f} & 1\end{matrix} ] \cdot [ \begin{matrix}1 & d \\ 0 & 1\end{matrix} ] . \qquad ( 30 )$$

Equation (30) reduces to Equation (31) as follows:

$$M = [ \begin{matrix}1 & z \\ 0 & 1\end{matrix} ] \cdot [ \begin{matrix}1 & d \\ - \frac{1}{f} & 1 - \frac{d}{f}\end{matrix} ] . \qquad ( 31 )$$

Equation (31) reduces to Equation (32) as follows:

$$M = [ \begin{matrix}1 - \frac{z}{f} & d + z - \frac{dz}{f} \\ - \frac{1}{f} & 1 - \frac{d}{f}\end{matrix} ] . \qquad ( 32 )$$

Considering the on-axis points, all of the heights will be zero. The point on the focal plane where different rays will strike is given by Equation (33) as follows:

$$( d + z - \frac{dz}{f} ) θ . \qquad ( 33 )$$

As shown above in (33), θ is the angle of the ray. The point in perfect focus is given by the lens maker's equation given in Equation (34) as follows:

$$\frac{1}{f} = \frac{1}{z} + \frac{1}{d} . \qquad ( 34 )$$

Equation (34) may be rearranged to derive Equation (35) as follows:

$$d = \frac{1}{\frac{1}{f} - \frac{1}{z}} = \frac{fz}{z - f} . \qquad ( 35 )$$

Inserting d from Equation (35) into Equation (33) to show the striking point results in Equation (36) as follows:

$$[ \frac{fx}{z - f} + z - \frac{\frac{fz}{z - f} z}{f} ] θ = \frac{{f}^{2} z + {fz}^{2} - {f}^{2} z - {fz}^{2}}{f ( z - f )} θ = 0. \qquad ( 36 )$$

All rays leaving this point strike the focal plane at the optical axis. As shown in Equation (37), the situation when the image sensor is shifted by a distance 4 from the focus is described as follows:

$$\begin{matrix}[ \frac{fx}{z - f} + δ + z - \frac{[ \frac{fz}{z - f} + δ ] z}{f} ] θ = \frac{\begin{matrix}{f}^{2} z + fz δ - {f}^{2} δ + {fz}^{2} - \\ {f}^{2} z - {fz}^{2} - δ {z}^{2} + f δ {z}^{2}\end{matrix}}{f ( z - f )} θ \\ = \frac{fz - {f}^{2} - {z}^{2} + fz}{f ( z - f )} δ θ \\ = - \frac{{( z - f )}^{2}}{f ( z - f )} δ θ \\ = \frac{f - z}{f} δ θ .\end{matrix} \qquad ( 37 )$$

Equation (37) shows that by properly positioning the lens of the image sensor with respect to the focal plane, we can change the depth of field. Additionally, the spot size depends upon the magnitude of the angle θ. This angle depends linearly on the aperture of the vision system created by the image sensor.

Additionally or alternatively, in accordance with some embodiments of the present disclosure, an image sensor may be implemented by adjusting for various parameters, including: the distance to the focus as it affects compactness, alignment, and sensitivity of the vision system to the environment; the field of view of the system; and the lens-focal plane separation as it affects the tolerances on alignment of the system and the sensitivity of the system to the environment.

Embodiments of the Flow Meter with or without Valves Connected Thereto

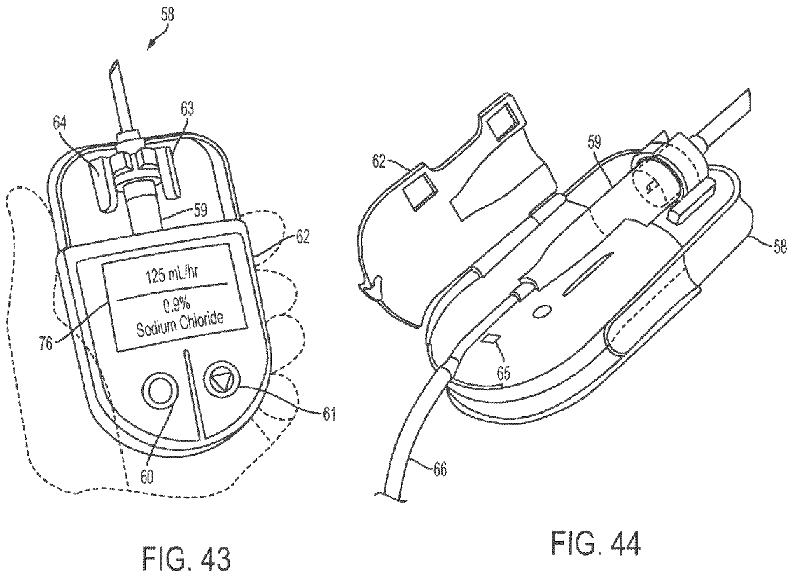

Referring to the drawings, FIGS. 43 and 44 show a flow meter 58 coupled to a drip chamber 59. As described infra, the flow meter 58 may optionally include a free flow detector component 12 (see FIG. 1) in accordance with an embodiment of the present disclosure. Additionally, alternatively, or optionally, the flow meter 58 may include a flow rate estimator component 13 (see FIG. 1) in accordance with some embodiments of the present disclosure. FIG. 43 shows the flow meter 58 with a shut door 62, and FIG. 44 shows the flow meter 58 with an open door 62. The flow meter 58 may be the flow meter 7 of FIG. 1 with a valve 6 or with no valve. The flow meter 58 includes a start button 60 and a stop button 61. Additionally or optionally, the flow meter 58 may include a backup valve to stop fluid from flowing therethrough or may signal another valve to stop the fluid from flowing in response to error conditions.

The flow meter 58 optionally includes image sensors 63 and 64 that can estimate fluid flow and/or detect free flow conditions. Although the flow meter 58 includes two image sensors (e.g., 63 and 64), only one of the image sensors 63 and 64 may be used in some embodiments. The image sensors 63 and 64 can image a drop while being formed within the drip chamber 59 and estimate its size. The size of the drop may be used to estimate fluid flow through the drip chamber 59. For example, in some embodiments of the present disclosure, the image sensors 63 and 64 use an edge detection algorithm to estimate the outline of the size of a drop formed within the drip chamber 59; a processor therein (see processor 15 of FIG. 1, processor 75 of FIG. 5, or processor 90 of FIG. 6 or 8) may assume the outline is uniform from every angle of the drop and can estimate the drop's size from the outline. In the exemplary embodiment shown in FIGS. 43 and 44, the two image sensors 63 and 64 may average together the two outlines to estimate the drop's size. For example, the algorithm may average the measured outlines of the two image sensor 63 and 64 to determine the size of the drop. The image sensors 63 and 64 may use a reference background pattern to facilitate the recognition of the size of the drop as described herein.

In another embodiment of the present disclosure, the image sensors 63 and 64 image the fluid to determine if a free flow condition exists. The image sensors 63 and 64 may use a background pattern to determine if the fluid is freely flowing (i.e., drops are not forming and the fluid streams through the drip chamber 59). As previously mentioned, although the flow meter 58 includes two image sensors (e.g., 63 and 64), only one of the image sensors 64 and 64 may be used in some embodiments to determine if a free flow condition exists and/or to estimate the flow of fluid through the drip chamber.

Additionally or alternatively, in some embodiments of the present disclosure, another image sensor 65 monitors the fluid tube 66 to detect the presence of one or more bubbles within the fluid tube. In alternative embodiments, other bubble detectors may be used in place of the image sensor 65. In yet additional embodiments, no bubble detection is used in the flow meter 58.

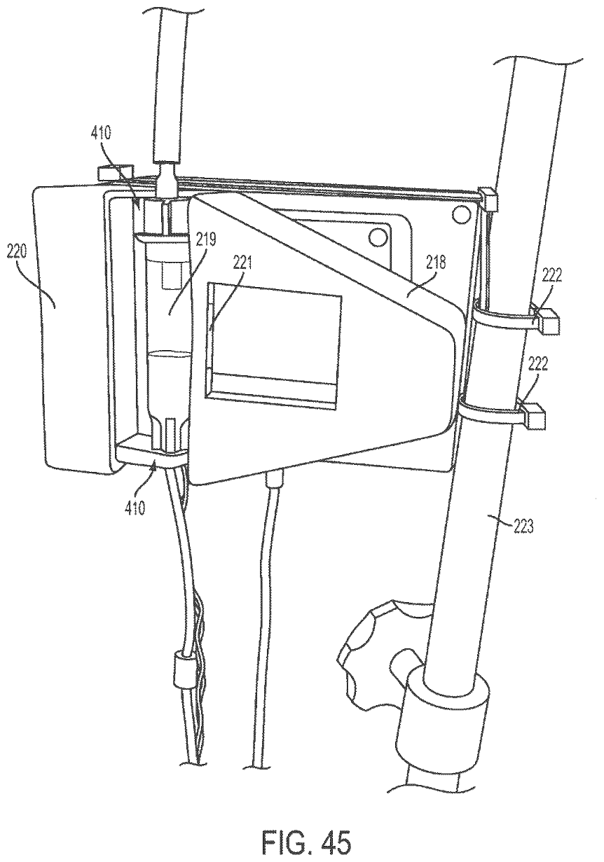

Referring now to the drawings, FIG. 45 shows a flow meter 218 coupled to a drip chamber 219 in accordance with an embodiment of the present disclosure. The drip chamber 219 is secured to the flow meter 218 via couplers 410. A backlight 220 shines light through the drip chamber toward the image sensor 221 (shown in outlined form).

The flow meter 218 may electronically transmit a flow rate to a monitoring client 8 (see FIG. 1). Additionally or alternatively, in some optional embodiments, the flow meter 218 may include a display that displays a flow rate (e.g., a touch screen, an LED display, and the like). The flow meter 218 may be coupled to a pole 223 via clamps 222.

In some embodiments, the flow meter 218 may be coupled to an actuator which is coupled to a valve (not shown in FIG. 45) to form a closed-loop system (e.g., the control component 14 of FIG. 1, such as a PID, bang-bang, neural network, or fuzzy logic control system) to regulate the flow of fluid through the drip chamber 219.

The flow meter 218 may use any flow algorithm described herein and may include any imaging system described herein. Additionally or alternatively, the flow meter 218 may include a free flow detector component (e.g., the free flow detector component 12 of FIG. 1).

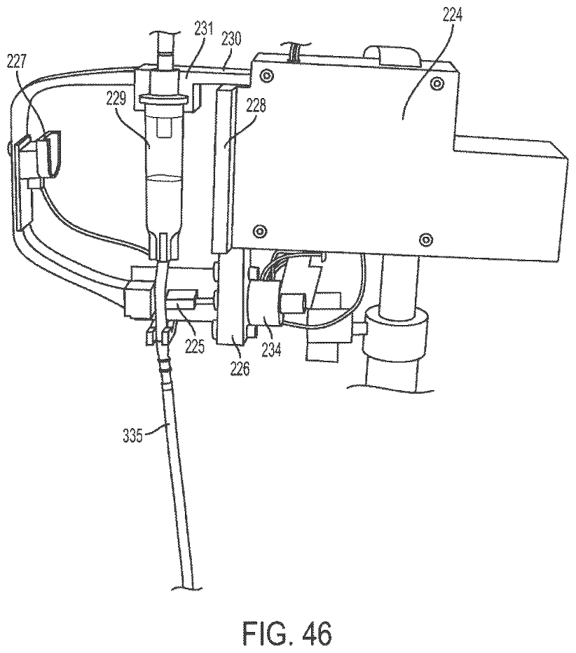

FIG. 46 shows a flow meter 224 and a pinch valve 225 coupled to the body 226 of the flow meter 224 to control the flow of fluid to a patient in accordance with an embodiment of the present disclosure. The flow meter 224 includes an image sensor 227 and a backlight 228.

The image sensor 227 images a drip chamber 229 and can receive illumination from the backlight 228. The flow meter 224 includes a support member 230 coupled to a coupler 231 that couples the drip chamber 229 to the flow meter 224.

The flow meter 224 may implement any flow rate estimator described herein (e.g., the flow rate estimator component 13 of FIG. 1) and/or a free flow detector disclosed herein (e.g., the free flow detector component 12 of FIG. 1). The flow meter 224 may use the pinch valve 225 in a close-loop fashion to control the flow of fluid to a patient (e.g., using a control component 14 as shown in FIG. 1).

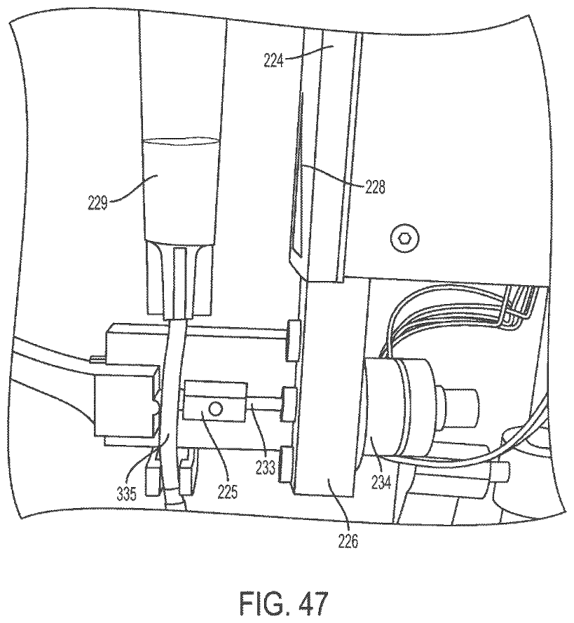

The pinch valve 225, as is more easily seen in FIG. 47, is coupled to a shaft 233 which is coupled to an actuator 234. The actuator 234 may be a solenoid or any actuator that can move the pinch valve 225 toward a tube 335.

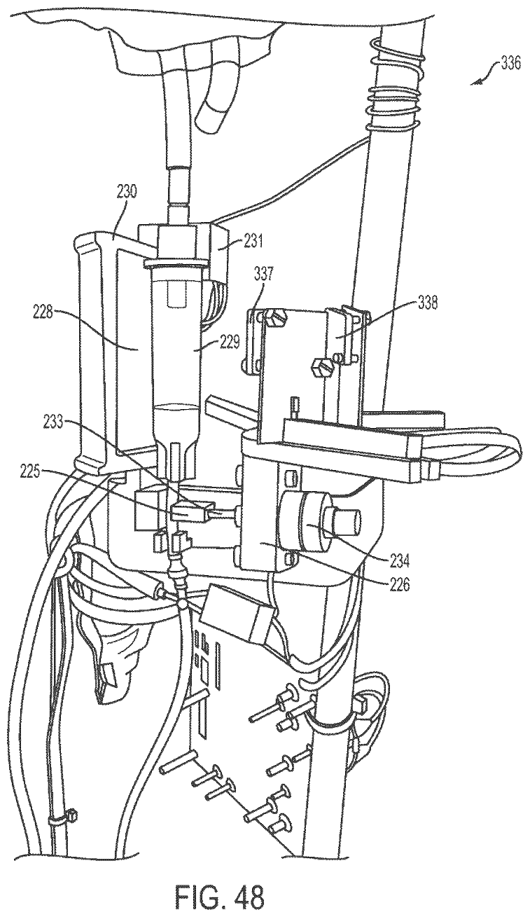

FIG. 48 shows a flow meter 336 and a pinch valve 225 in accordance with an embodiment of the present disclosure. The flow meter includes two image sensors 337 and 338. The flow meter 336 may use the pinch valve 225 in a closed-loop feedback configuration. The flow meter 336 may implement a volume estimation algorithm described herein using both image sensors 337 and 338 to estimate the flow of fluid through the drip chamber 229. For example, the flow meter 336 may average the two volumes together for use in the feedback loop.

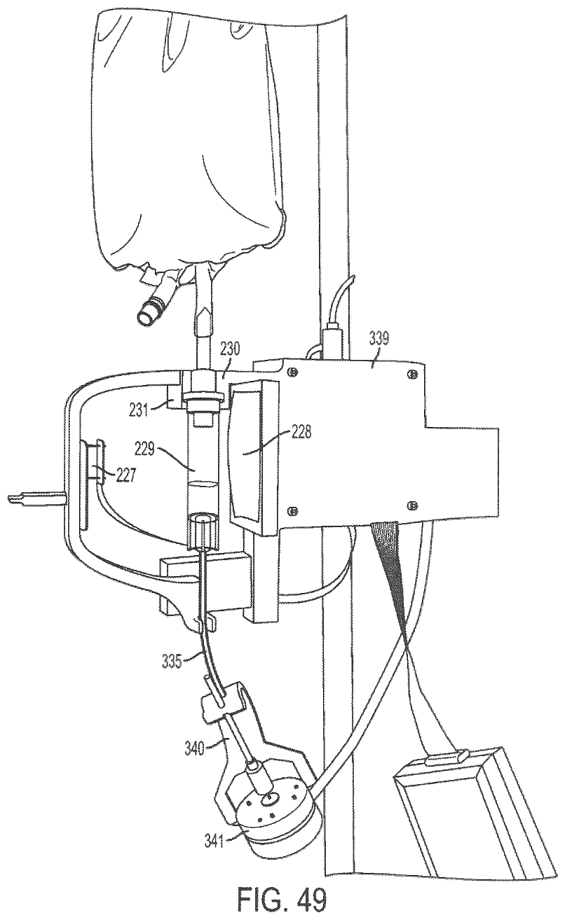

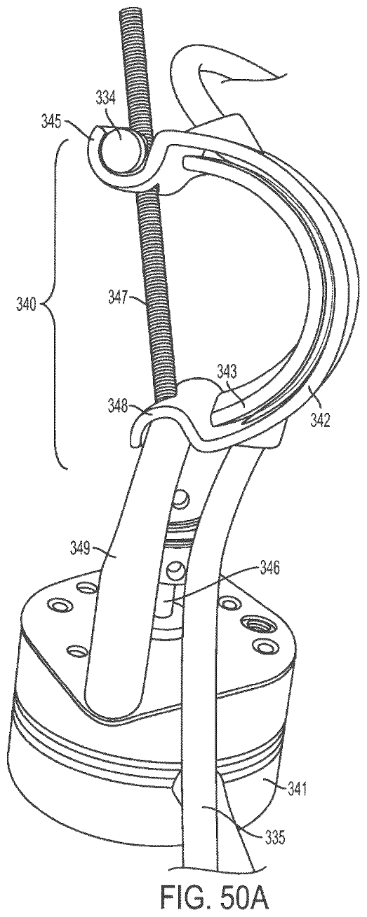

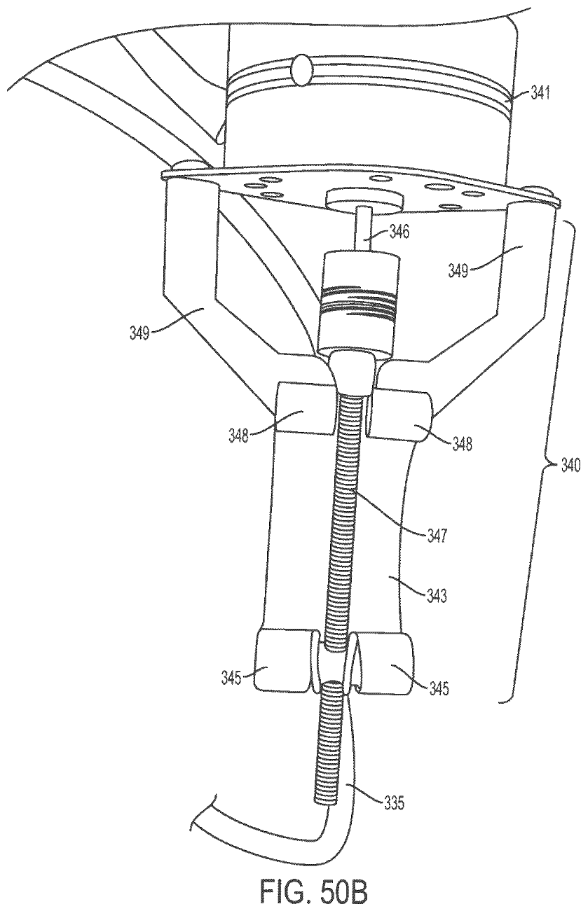

FIG. 49 shows a flow meter 339 and a valve 340 coupled to an actuator 341 to control the flow of fluid into a patient in accordance with an embodiment of the present disclosure. The flow meter 339 of FIG. 49 is similar to the flow meter 224 of FIG. 46; however, the flow meter 339 of FIG. 49 includes a valve 340 that has curved, elongated support members 342 and 343 (see FIGS. 50A-50B).

The flow meter 339 includes an image sensor 227 and a backlight 228. The image sensor 227 images a drip chamber 229 and can receive illumination from the backlight 228. The flow meter 339 includes a support member 230 coupled to a coupler 231 that couples the drip chamber 229 to the flow meter 339.

The flow meter 339 can implement any flow rate estimator described herein (e.g., the flow rate estimator component 13 of FIG. 1) and/or a free flow detector disclosed herein (e.g., the free flow detector component 12 of FIG. 1). The flow meter 339 may use the valve 340 in a close-loop fashion to control the flow of fluid into a patient (e.g., using the control component 14 of FIG. 1).

The flow meter 339 may actuate the actuator 341 to actuate the valve 340, which thereby regulates the fluid flowing through the IV tube 335 in a feedback (i.e., closed-loop) configuration using any control algorithm.

Referring now to FIGS. 50A-50B, which shows close-up views of the valve 340 of FIG. 49 in accordance with an embodiment of the present disclosure. The valve 340 includes an inner curved, elongated support member 343 and an outer curved, elongated support member 342. The tube 335 is positioned between the support members 342 and 343.

The inner support member 343 includes a barrel nut 344. The outer support member 342 is coupled to the barrel nut 344 via hooks 345. In some embodiments, the barrel nut 344 is not coupled to the valve 340 and the inner support member 342 includes a hole for the threaded rod or screw 347 to slide through. The outer support member 342 also has hooks 348 to secure it to a frame 349 of the actuator 341. The actuator 341 includes a shaft 346 coupled to a screw 347. As the actuator 341 rotates the shaft 346, the screw 347 can rotate to push the barrel nut 334 toward the actuator 341. That is, the hooks 345 and the barrel nut 334 move toward the hooks 348 and the frame 349 because the inner and outer support members 342 and 343 are flexible.

As the support members 342 and 343 are compressed, the tube 335 becomes compressed because it is positioned between the support members 342 and 343. Compression of the tube 335 restricts the flow of fluid through the tube 335. The valve 340 compresses a length of the tube 335 that is substantially greater than the diameter of the tube 335.

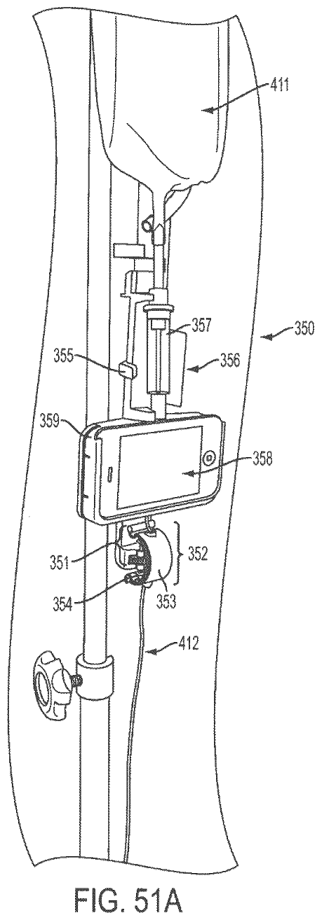

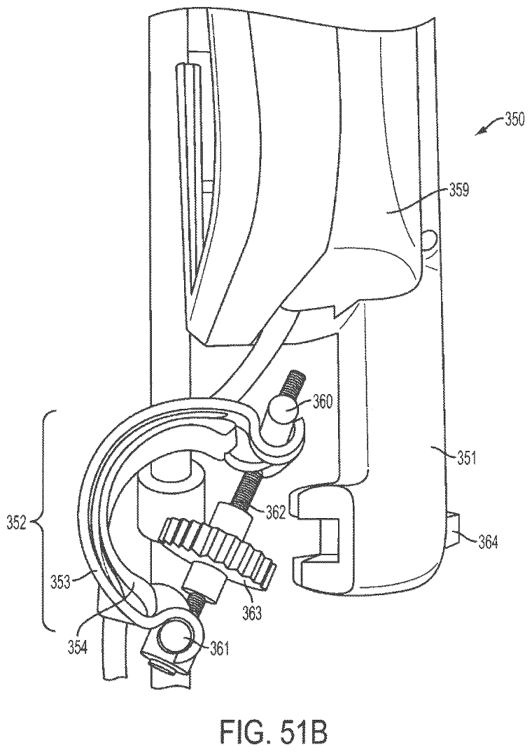

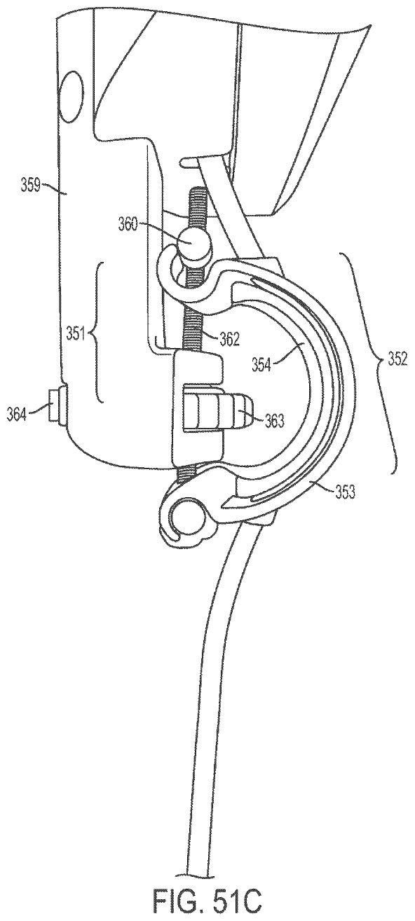

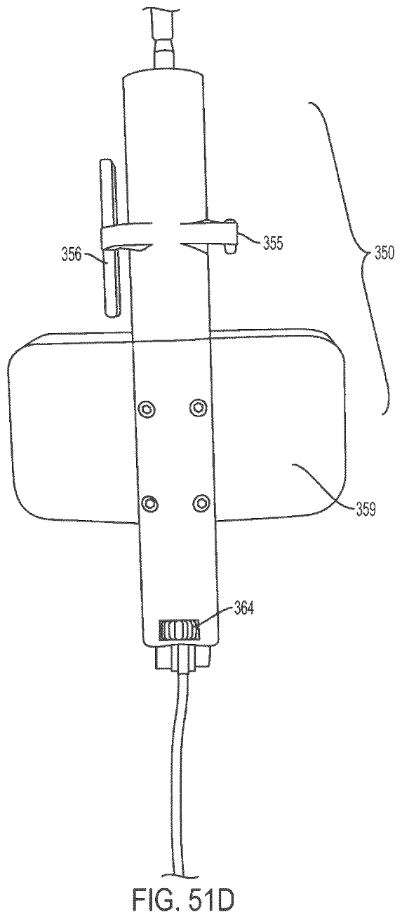

FIGS. 51A-51D show several views of a flow meter 350 with a monitoring client 358, a valve 352, a drip chamber 357, an IV bag 411, and a fluid tube 412 in accordance with an embodiment of the present disclosure. The flow meter 350 includes a receiving portion 351 to receive the valve 352. The valve 352 includes two curved, elongated support members 353 and 354.

The flow meter 350 includes an image sensor 355 and a backlight 356 that can monitor drops formed within the drip chamber 357. The flow meter 350 may use the image sensor 355 to implement a flow rate estimator algorithm described herein (e.g., the flow rate estimator component 13 of FIG. 1) and/or to implement a free flow detector disclosed herein (e.g., the free flow detector component 12 of FIG. 1).

The flow meter 350 includes a base 359 that can form a dock to receive the monitoring client 358. The monitoring client 358 may be a smart phone, or other electronic computing device (e.g., an Android-based device, an Iphone, a tablet, a PDA, and the like).

The monitoring client 358 may contain software therein to implement a free flow detector, a flow rate estimator, a control component, an exposure component, etc. (e.g., the free flow detector component 12, the flow rate estimator component 13, the control component 14, the exposure component 29 of FIG. 1) and may contain one or more transceivers (e.g., the transceiver 9). Additionally or alternatively, the base 359 of the flow meter 350 may implement these items.

For example, the flow meter 350 may implement a free flow detector, a flow rate estimator, a control component, an exposure component, etc. using internal software, hardware, electronics, and the like. The flow meter 350 may implement a closed-loop feedback system to regulate the fluid flowing to a patient by varying the fluid flowing through the valve 352.

As is easily seen in FIG. 51B, the valve 352 includes an inner support member 354 and an outer support member 353. The inner support member 354 is coupled to a barrel nut 360 and to a barrel 361. In some embodiments, the barrel nut 360 is not coupled to the inner support member 354, and the inner support member 354 includes a hole for the threaded shaft 362 to slide through.

A threaded shaft 362 (e.g., a screw) spins freely within a bearing located within the barrel 361 and engages a threaded nut within the barrel nut 360 to push or pull the barrel nut 360 relative to the barrel 361 by rotation of the knob 363 (e.g., the actuator is a lead screw having a knob to actuate the lead screw.). The knob 363 may be manually rotated.

Additionally or alternatively, the valve 352 may be snapped into the receiving portion 351 which includes a rotating member 364 that engages the knob 363 within the receiving portion 351 (see FIG. 51C). The rotating member 364 engages the rotating knob 363 to actuate the valve 352. The rotating member 364 may be coupled to an electric motor which rotates the rotating member 364. The electric motor (not explicitly shown) may be controlled by the flow meter 350 in a closed-loop configuration to achieve a target flow rate of fluid flowing into a patient.

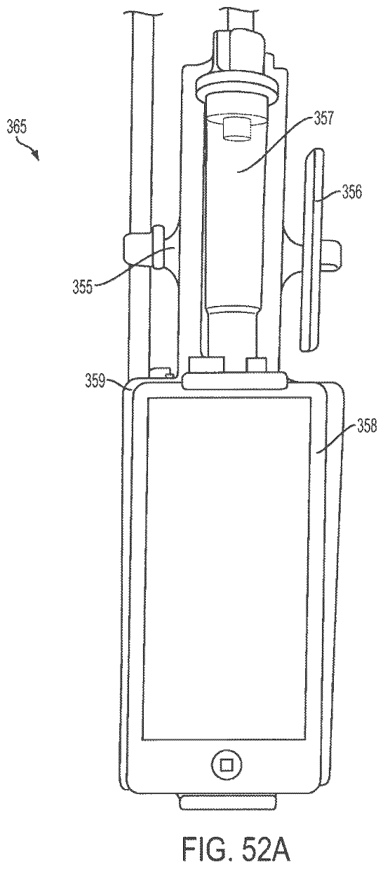

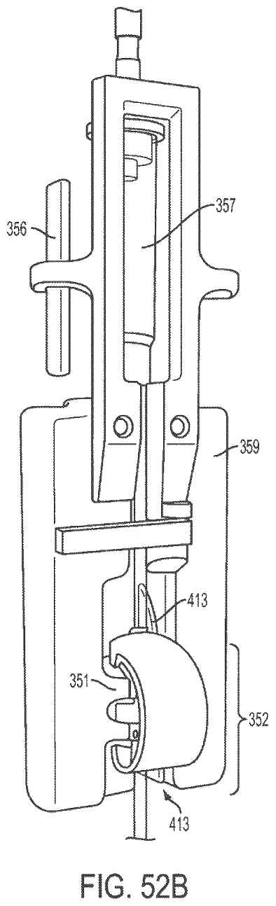

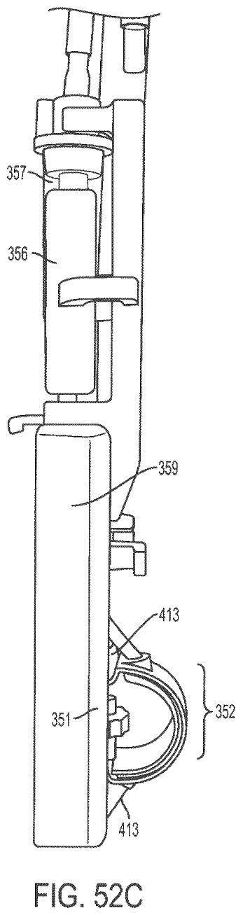

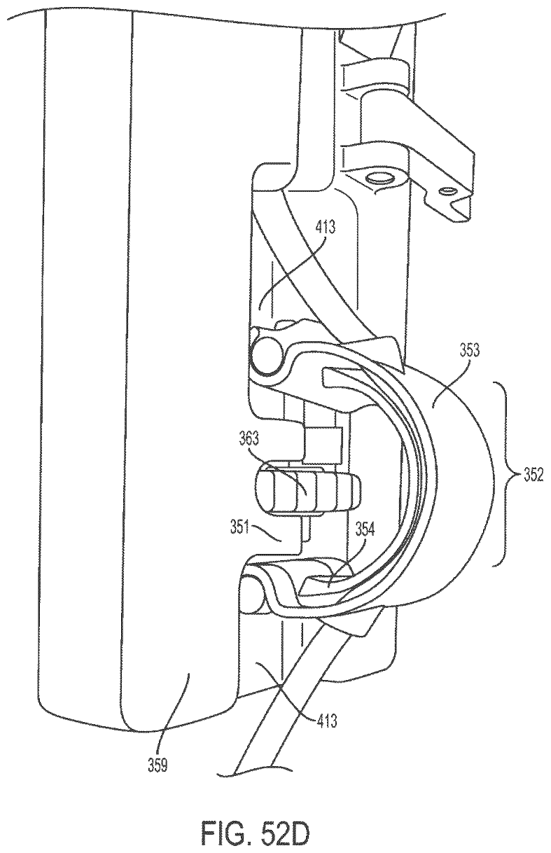

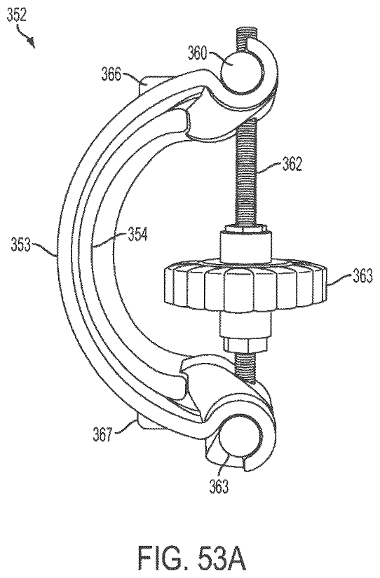

FIGS. 52A-52D show several views of another flow meter 365 with a valve 352, a drip chamber 357, and a fluid tube trench 413 having a receiving portion 351 to receive a valve 352 in accordance with an embodiment of the present disclosure. The flow meter 365 of FIGS. 52A-52D is similar to the flow meter 350 of FIGS. 5A-5D; however, the base 359 holds the monitoring client 358 in an “upright” position. Additionally, the receiving portion 351 is on an opposite side of the base 359 from the monitoring client 358 (see FIGS. 52B and 52C).

FIG. 52D shows a close-up view of the valve 352 engaging the receiving portion 351. The knob 363 engages a rotating member that is internal to the base 359 (not shown in FIG. 52D) that is coupled to a motor (also not shown in FIG. 52D).

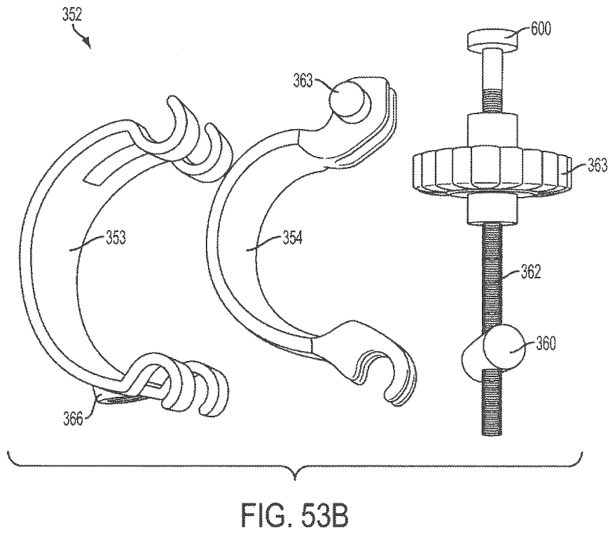

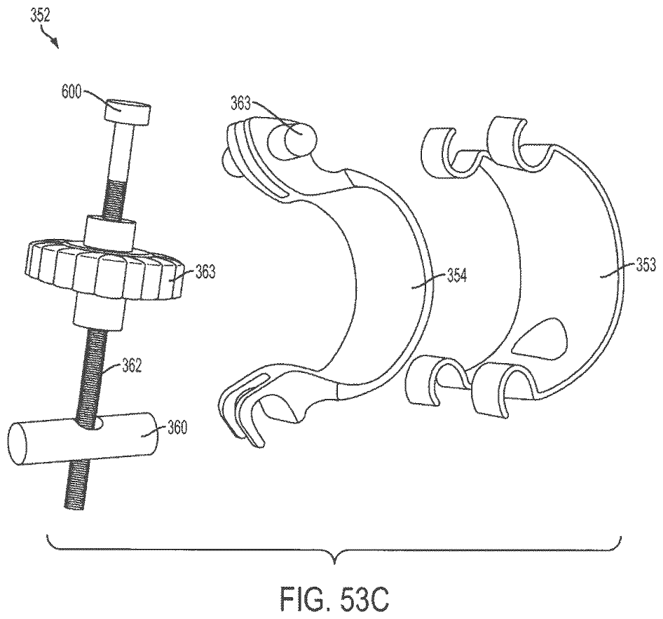

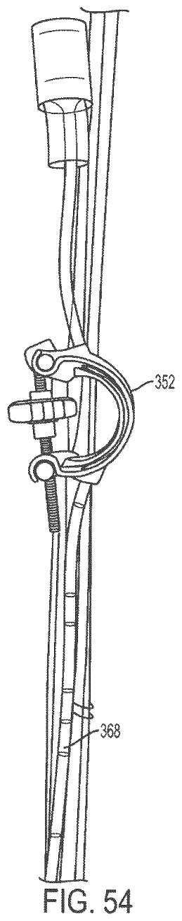

FIG. 53A shows another view of the valve 352 of FIGS. 51A-51D and 52A-52D, and FIGS. 53B-53C show two exploded views of the valve of FIG. 53A in accordance with an embodiment of the present disclosure.

As shown in FIGS. 53A-53C, the valve 352 includes an inner support member 354 and outer support member 353. A tube may be inserted through holes 366 and 367 to position the tube between the support members 354 and 353.

The knob 363 may be turned to turn the screw 362. Rotation of the screw 362 causes the barrel nut 360 to move toward the partial barrel 363 to compress a tube positioned between the support members 353 and 354. The partial barrel 363 includes two sides, however, there is a space to hold the end 600 (e.g., the cap) of the screw 362 securely within the space (e.g., a complementary space). FIG. 54 shows the valve 352 in manual use and coupled to a tube 368.

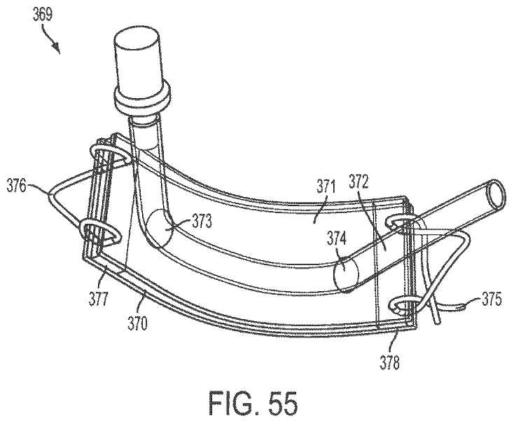

FIG. 55 shows a valve 369 that includes two flexible members 370 and 371 in accordance with an embodiment of the present disclosure. The flexible members 370 and 371 may be two flexible sheets. The flexible member 371 may include holes 373 and 374 for a tube 372 to be positioned between the flexible members 370 and 371.

The flexible members 370 and 371 are coupled together via two connector members 377 and 378. The connector members 377 and 378 are coupled to coupling members 376 and 375, respectively.

Actuation of the valve 369 may be by a linear actuator that pulls the coupling members 375, 376 toward each other or away from each other. The linear actuator (not explicitly shown) may be a screw-type actuator, a piston actuator, or other actuator. In some embodiments, one of the coupling members 375 and 376 may be coupled to a stationary support while the actuator is coupled to the other one of the coupling members 375 and 376 and another stationary support for pulling the coupling members 375 and 376 together or apart.

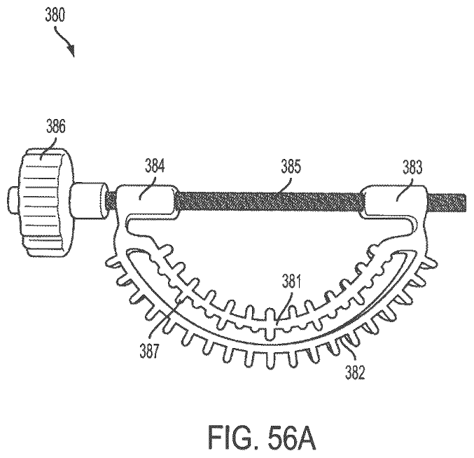

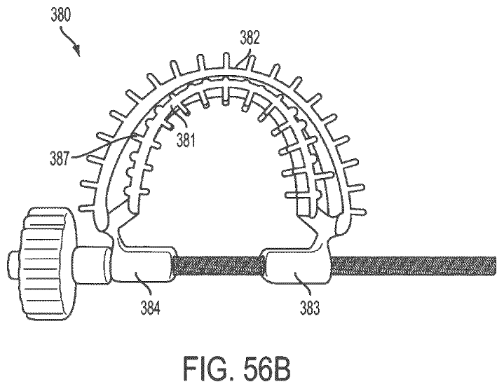

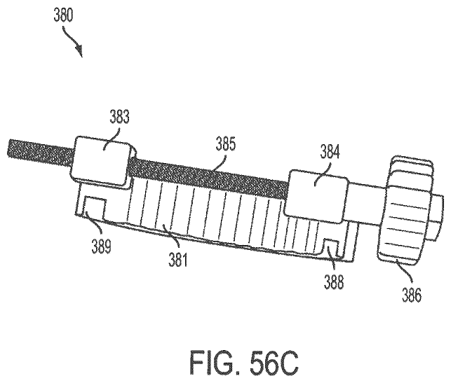

FIGS. 56A-56C show several views of a valve 380 having two curved, elongated support members 381 and 382 with one of the elongated support members 381 having a plurality of ridges 387 adapted to engage a tube positioned between the support members 381 and 382, in accordance with an embodiment of the present disclosure.

The valve 380 has both support members 381 and 382 coupled to a coupling member 383 at a first end and a second coupling member 384 at another end. That is, the coupling member 384 surrounds a screw 385, and the coupling member 383 includes internal threads for pulling the coupling member 383 toward or away from a knob 386 when the screw 385 is rotated with rotation of the knob 386. FIG. 56B shows the valve 380 when actuated to close fluid flowing through a tube coupled between the support members 381 and 382. FIG. 56C shows the support member 381 having two holes 388 and 389 to receive a tube. Also note that the support members 381 and 382 hold a tube off center from an axis of the screw 385, which is easily seen in FIG. 56C. Holding the tube off-center from the screw's 385 axis facilitates free movement of the tube.

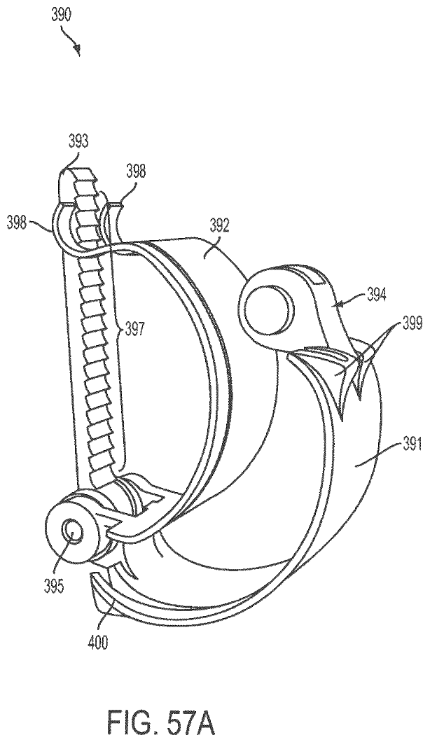

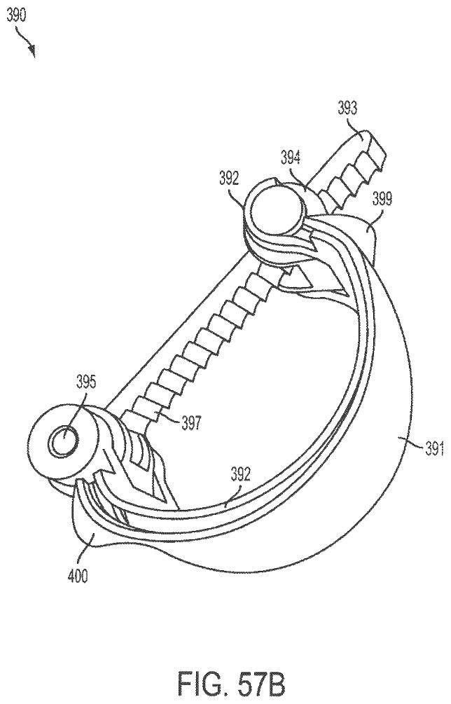

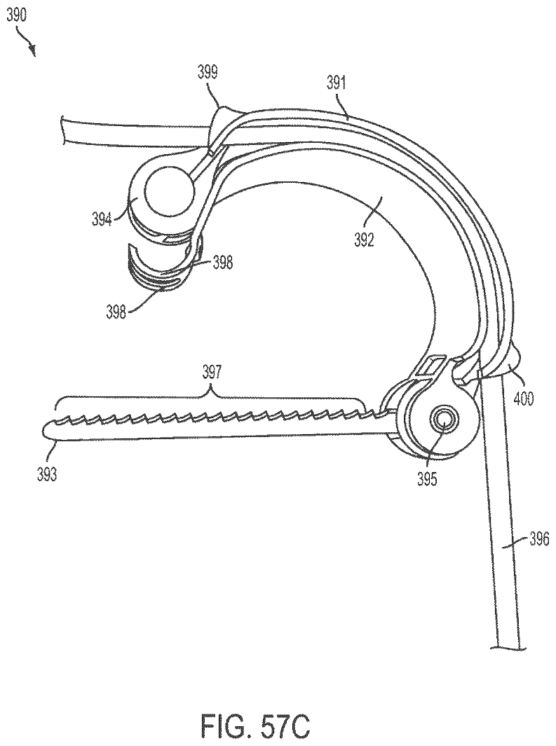

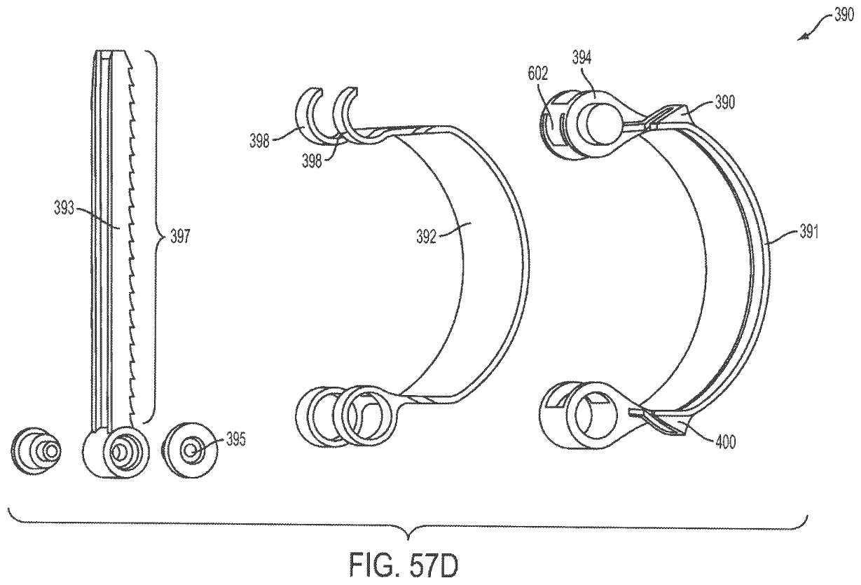

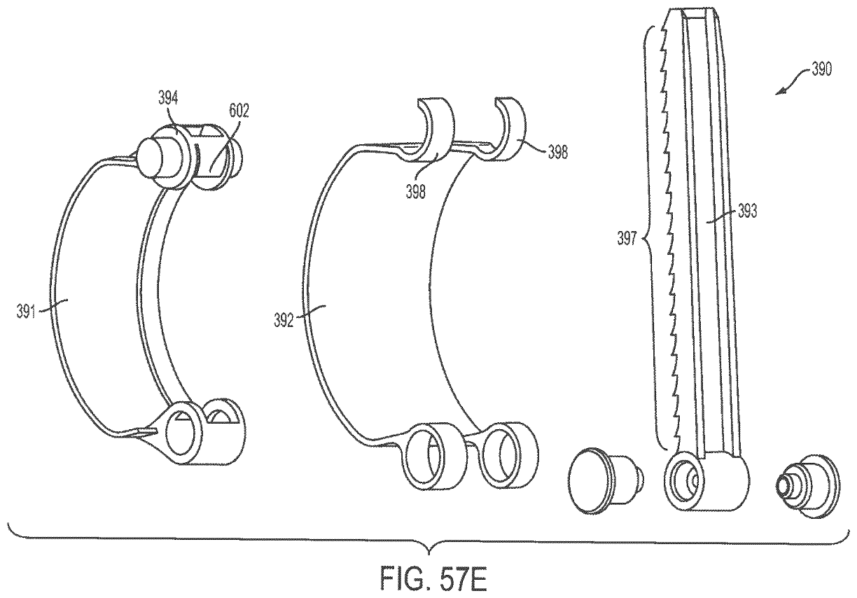

FIGS. 57A-57C show several views of a valve 390 having a ratchet 394 that engages a connecting member 393 of the valve 390 in accordance with an embodiment of the present disclosure, and FIGS. 57D-57E show two exploded views of the valve 390 of FIGS. 57A-57C. The ratchet 394 engages the connecting member 393 by interacting with a gear rack 397 disposed thereon. A finger 602 (see FIGS. 57D and 57E) interacts with a gear rack 397 to provide the ratcheting action. That is, the finger 602 may hold the gear rack 397 against an engaging finger on a side opposite of the retaining finger 602. The valve 390 includes a support member 391 having an end coupled to the ratchet 394 and another end pivotally coupled to a hinge 395. The valve 390 also includes a support member 392 having hooks 398 that can couple to the body of the ratchet 394.

As shown in FIG. 57C, a tube 396 can be positioned between the support members 391 and 392, the hooks 398 can then be fastened to the body of the ratchet 394, and the connecting member 393 can be inserted into the ratchet 394 (as shown in FIG. 57B). As shown in FIG. 57C, the tube 396 is positioned against the support member 391 via openings 399 and 400.

The ratchet 394 engages the gear rack 397 such that the ratchet 394 can be manually moved toward the hinge 395 for course fluid flow adjustments. Thereafter, a knob (not shown) may be coupled to the ratchet 394 to make fine adjustments to the distance between the ratchet 394 and the hinge 395. Additionally or alternatively, the ratchet 394 may include a release button (not shown) to release the ratchet from the connecting member 393.

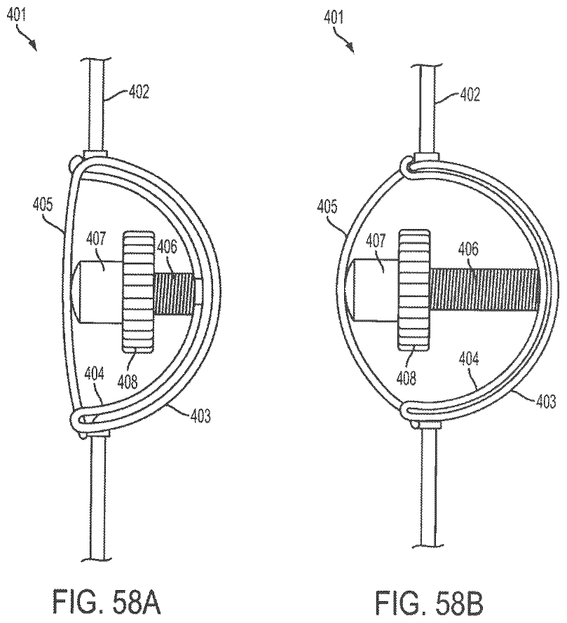

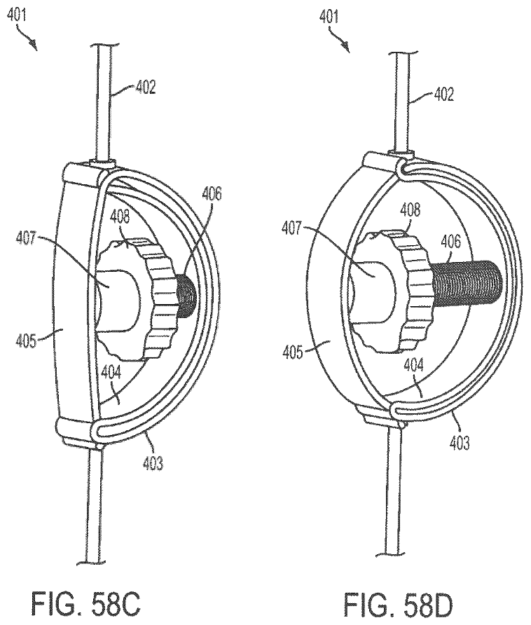

FIGS. 58A-58D show several views of a valve 401 having two elongated support members 403 and 404, a connecting member 405, and a screw-type actuator 407 in accordance with another embodiment of the present disclosure.

The support members 403 and 404 may be permanently molded together at their ends with the ends of the connecting member 405. A tube 402 may be positioned between the support members 403 and 404.

As the knob 408 is turned, the screw-type actuator 407 expands or contracts because of engagement with a threaded rod 406. FIG. 58A shows the valve in an open position while FIG. 58B shows the valve in a closed position. Note that the tube 402 is squeezed along a substantial length of the tube 402. FIGS. 58C-58D show the valve 401 in the open position and the closed position, respectively, from a perspective view.

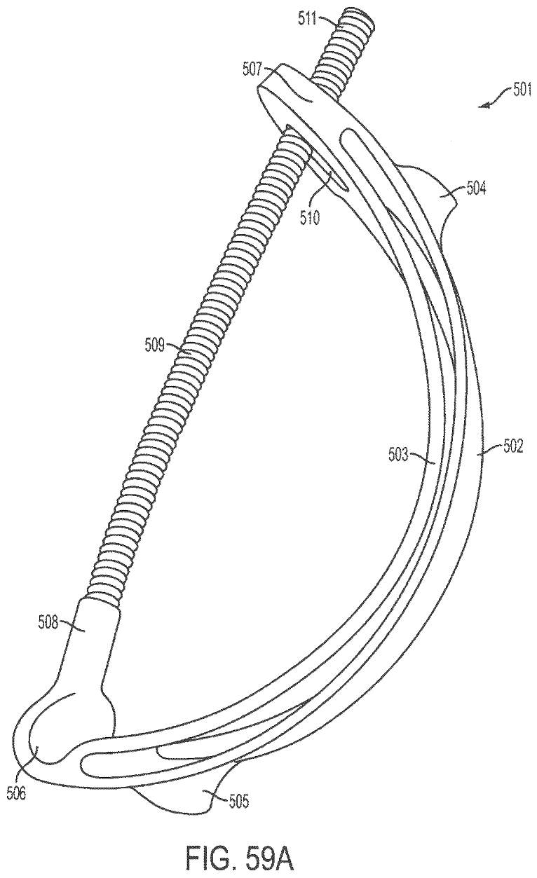

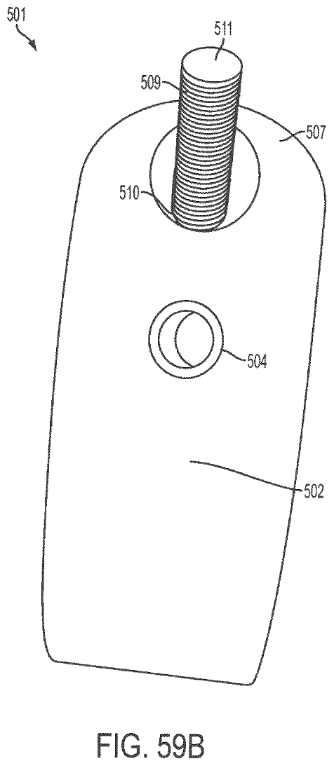

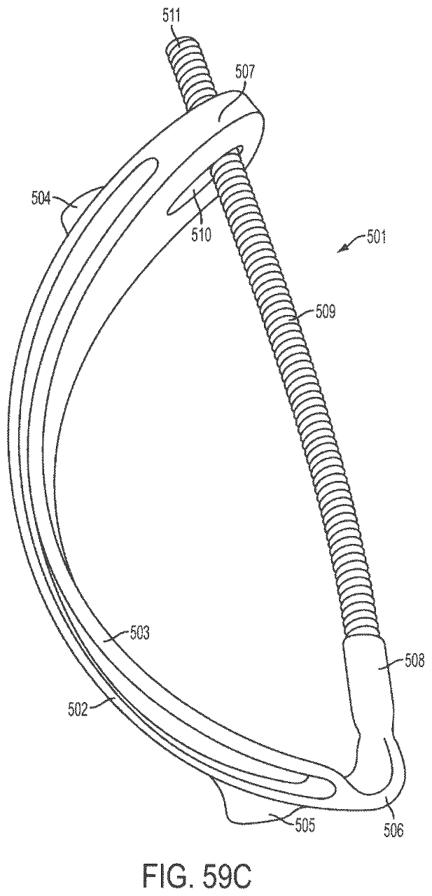

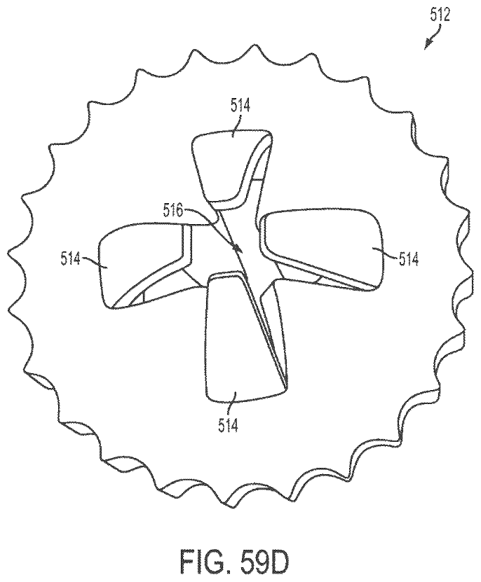

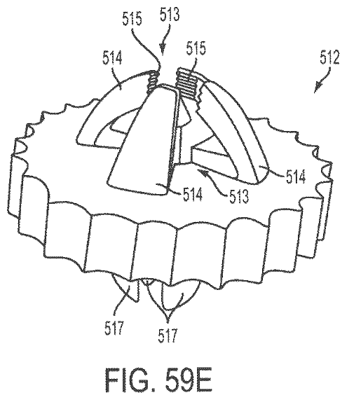

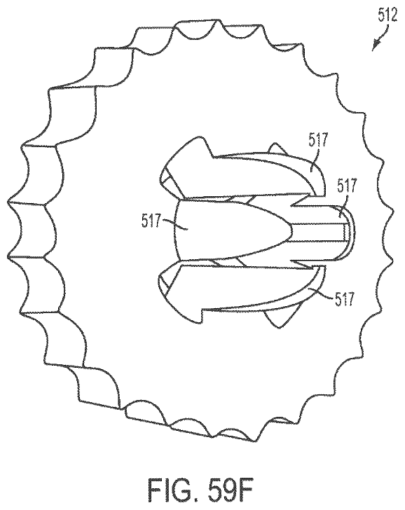

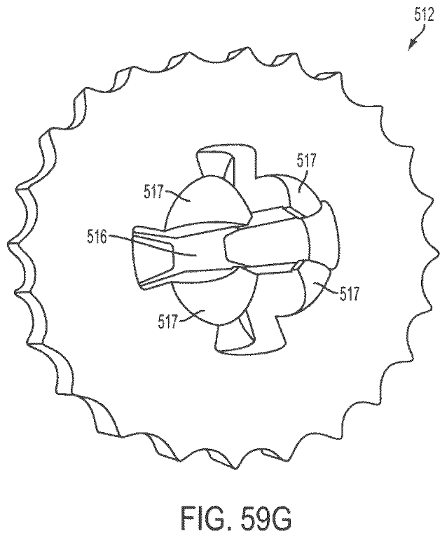

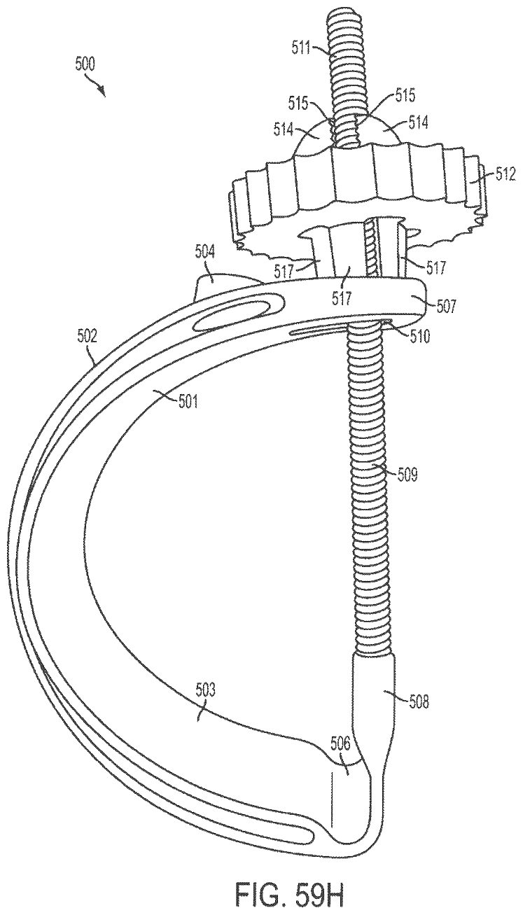

FIGS. 59A-59C show several views of a body 501 of a valve 500 (see FIG. 59H for the assembled valve 500) in accordance with an embodiment of the present disclosure. The body 501 includes a first curved, elongated support member 502 and a second curved, elongated support member 503. The first support member 502 includes raised holes 504, 505 to hold a tube between the support members 502 and 503.

The body 501 also includes a first connector 506 that is coupled to the support members 503, 504 at an end, and a second connector 507 that is coupled to the other ends of the support members 503, 504.

The first connector 506 is coupled to an end of the support members 503, 504 and to a first end 508 of a connecting member 509. The second connector 507 includes a hole 510 for positioning the second end 511 of the connector member 509 therethrough (as is easily seen in FIG. 59B).

When a tube is positioned between the support members 502, 503, movement of the second connector 507 toward the first connector 506 compresses the tube disposed between the support members 502, 503. As the second connector 507 moves towards the first connector, the hole 510 of the second connector 507 allows the second end 511 of the connector member 509 to freely slide therein.

FIGS. 59D-59G show several views of a knob 512 for use with the body 501 shown in FIGS. 59A-59C in accordance with an embodiment of the present disclosure. The knob 512 includes a ratchet 513 defined by four fingers 514. Each of the fingers 514 includes a threaded surface 515 to engage a threaded connecting member 509. The fingers 514 are arched toward a hole 516 at the center of the knob 512. The knob 512 also includes fingers 517 that engage the second connector 507 (see FIG. 59H). In some embodiments, the body 501 includes a recess 510 to receive the fingers 517 on the second connector 508.

FIG. 59H shows an assembly valve 500 that includes the body 501 shown in FIGS. 59A-59C coupled to the knob 512 of FIGS. 59D-59G in accordance with an embodiment of the present disclosure. The knob 512 is slid onto the threads of the connecting member 509. The fingers 514 engage the threads of the connecting member 509 and ratchet onto the connecting member 509. That is, the knob 512 is freely moveable towards the first end 508 of the connecting member 509 along the threads of the connecting member 509, but cannot be moved away from the first end 508 of the connecting member 509 without rotating the knob 512. That is, the knob 512 may be placed onto the connecting member 509 to provide a coarse adjustment of the valve 500 by coarsely moving the connectors 507, 508 toward each other to close the valve 500. Because the threaded surfaces 515 of the four fingers 514 engage the threads of the connecting member 509, rotation of the knob 512 either reduces or increases fluid flow within a tube. Each of the fingers 514 includes a threaded surface 515 to engage the threads of the connecting member 509 such that rotation of the knob 512 moves the second connector 507 toward or away from the first connector 506 to thereby control the flow of fluid of a tube positioned between the support members 502, 503.

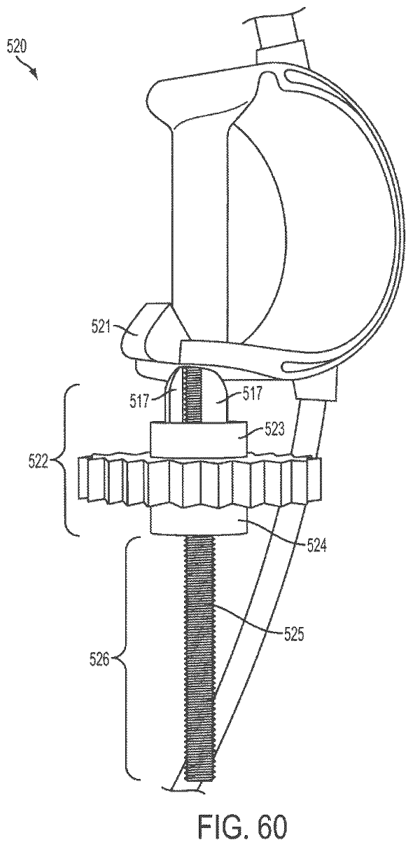

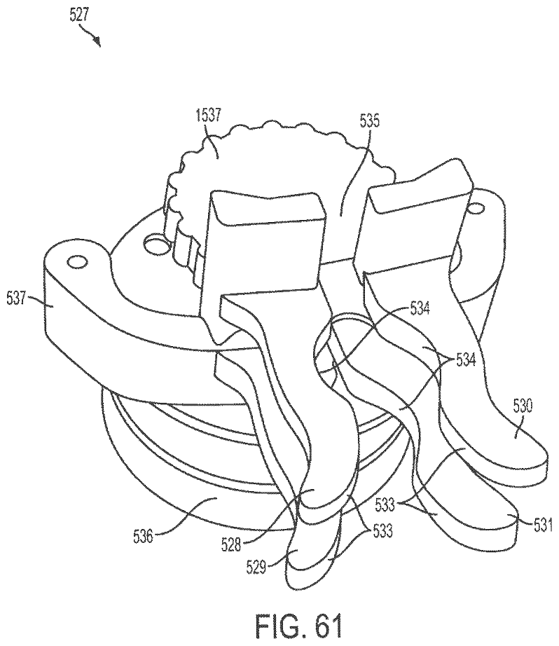

FIG. 60 shows a valve 520 having a guiding protrusion 521 in accordance with an embodiment of the present disclosure. The valve 520 is similar to the valve 500 of FIG. 59H, but includes the guiding protrusion 521 and a knob 522 having first and second collars 523, 524. The knob 522 also includes internal threads (not shown) to engage threads 525 of a connecting rod 526. In some embodiments, the internal threads may be ratcheting, and in other embodiments, the internal threads may be fixed without providing a ratcheting action.

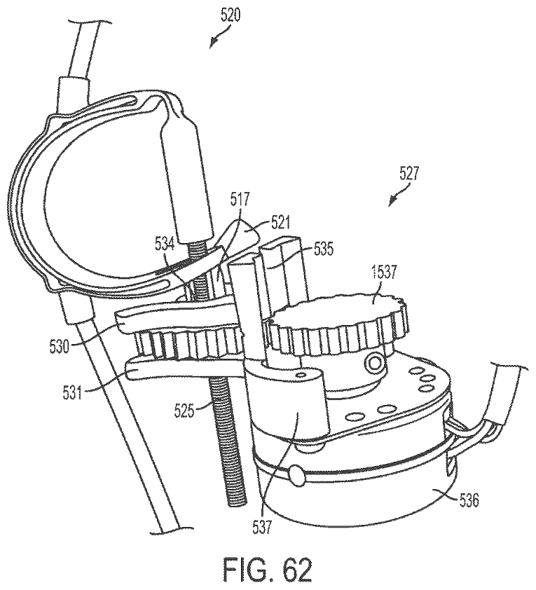

FIG. 61 shows a motor 536 and a valve-securing structure 537 for coupling to the valve 520 of FIG. 60 in accordance with an embodiment of the present disclosure. The valve-securing structure 537 includes securing fingers 528, 529, 530, 531 each having a curved portion 533 for snapping onto collars 523, 524 of a knob 522 (see FIG. 62) into respective collar-guiding portions 534.

Referring now to FIGS. 60, 61, and 62, once the collars 523, 524 are sufficiently secured, the knob 522 is free to rotate. That is, the collar 523 may be secured between the securing fingers 528 and 530 within their respective collar-guiding portion 534 allowing the knob 522 to rotate. Likewise, the collar 524 may be secured between the securing fingers 529 and 531 within their respective collar-guiding portion 534 allowing the knob 522 to rotate.

When the valve 520 is secured to the valve-securing structure 537, rotation of the wheel 1537 (caused by the motor 536) rotates the knob 522 of the valve 520. As the valve 520 flexes, the protrusion 521 freely moves within the protrusion guide 535 or adjacent to the protrusion guide 535. FIG. 62 shows the valve of FIG. 60 secured to the motor 536 via the valve-securing structure 537.

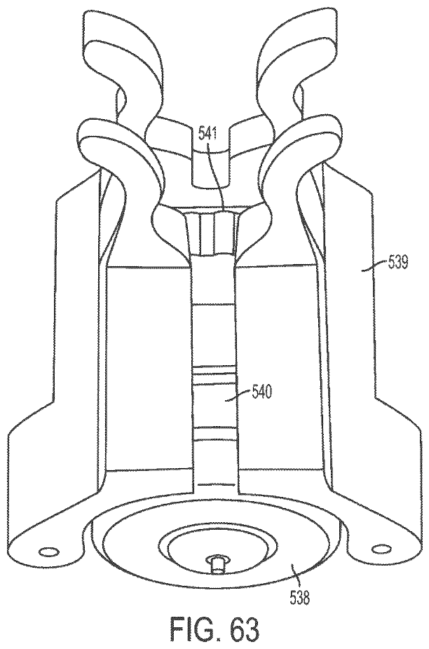

FIG. 63 shows another motor 538 and valve-securing structure 539 for coupling to the valve of FIG. 60 in accordance with an embodiment of the present disclosure. The valve-securing structure 539 includes a protrusion guide 540 adjacent to the motor 538. The motor 538 is coupled to the wheel 541 to engage the knob 522 (see FIG. 60).

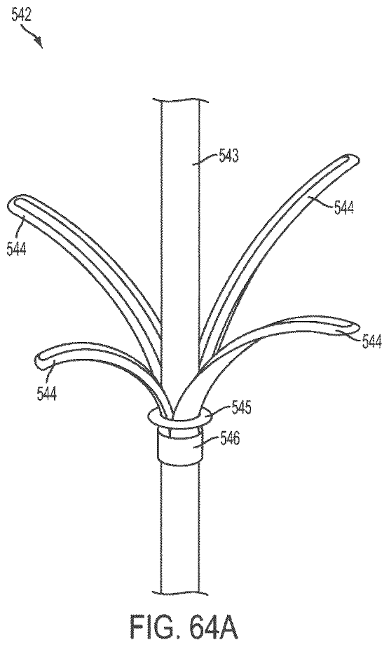

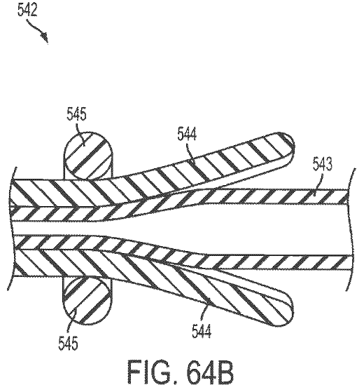

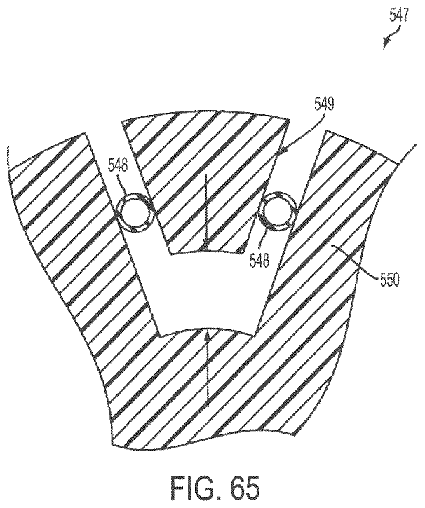

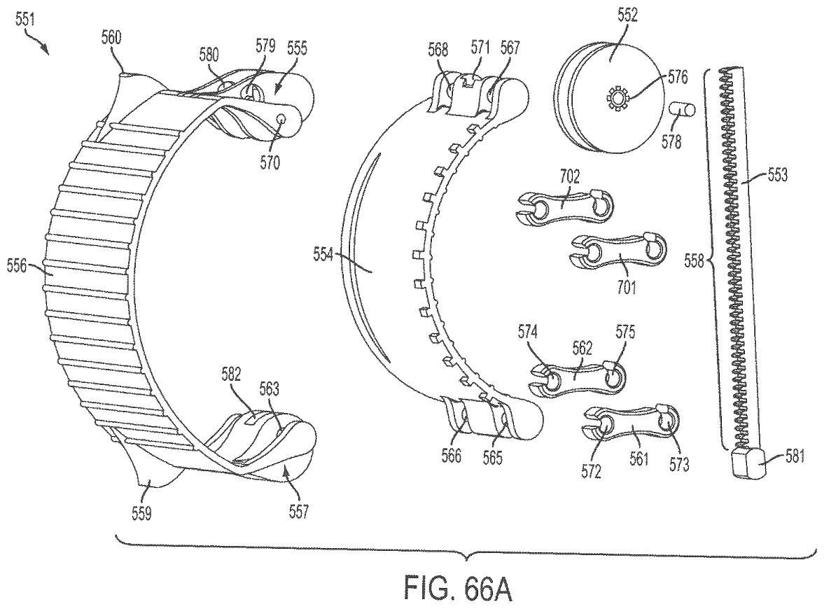

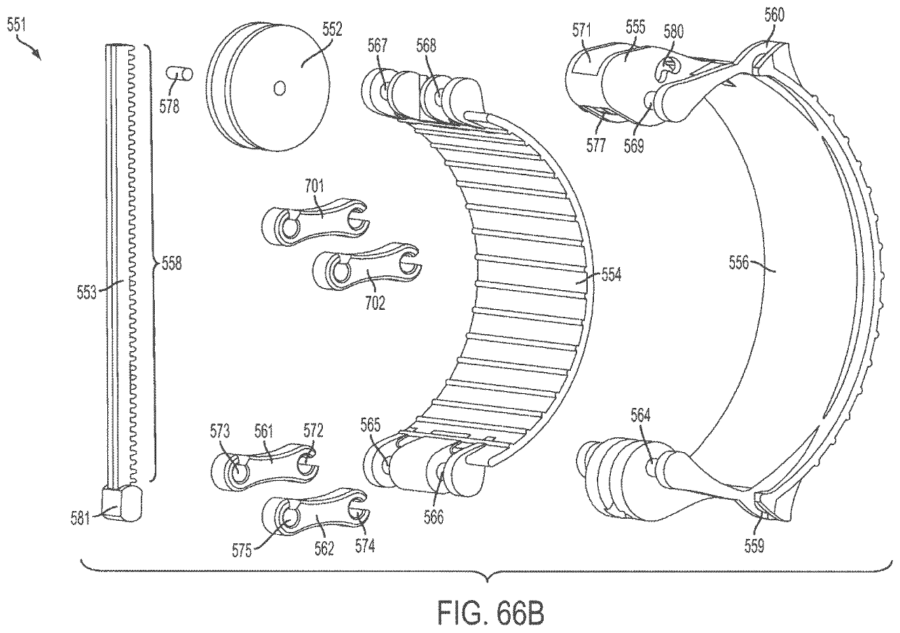

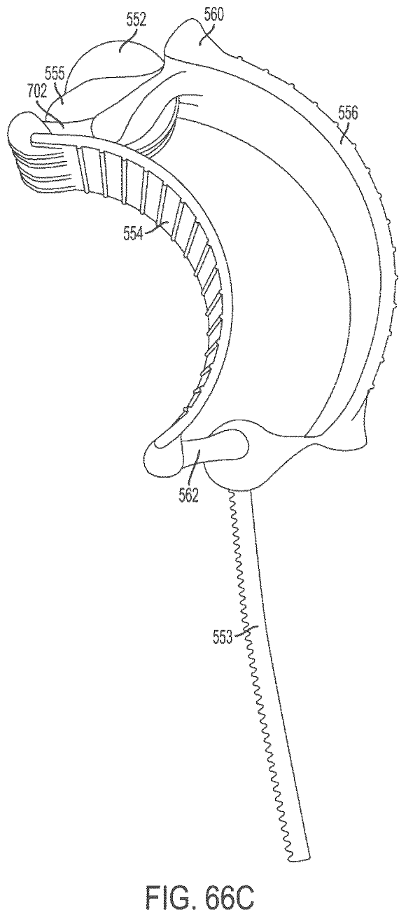

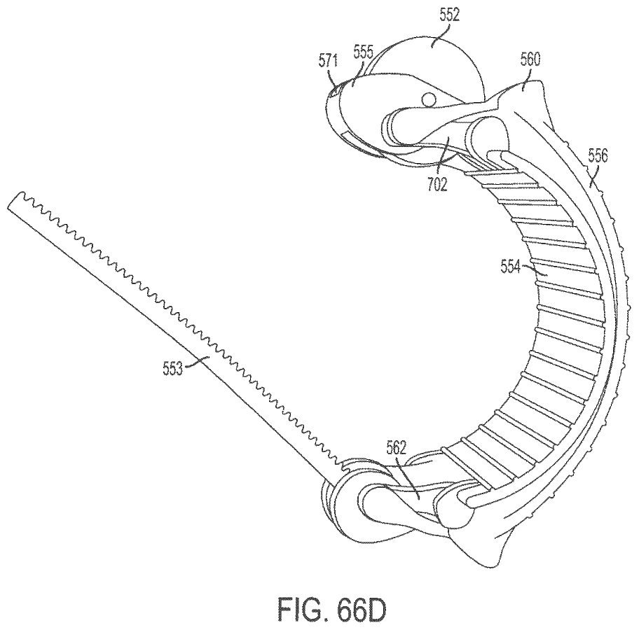

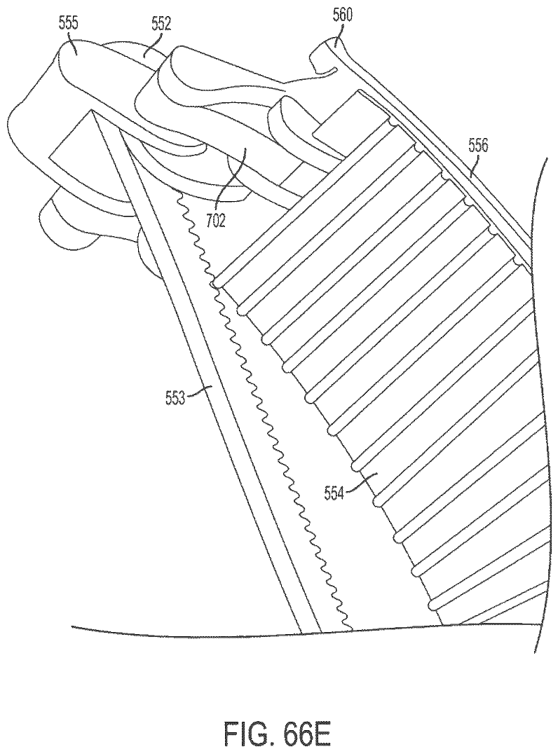

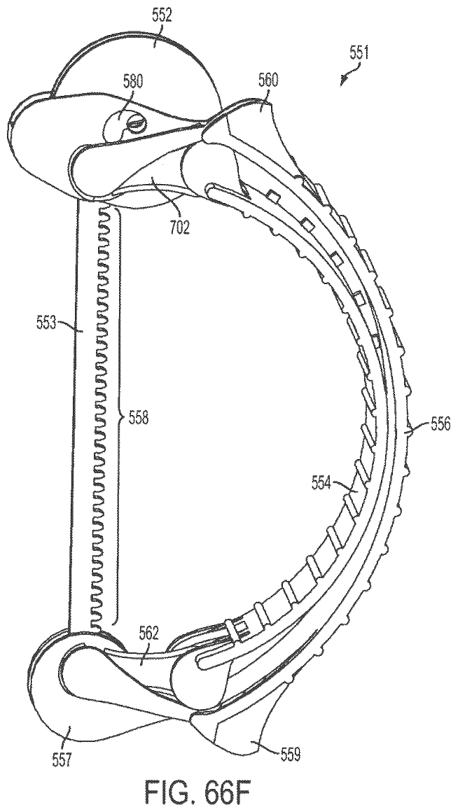

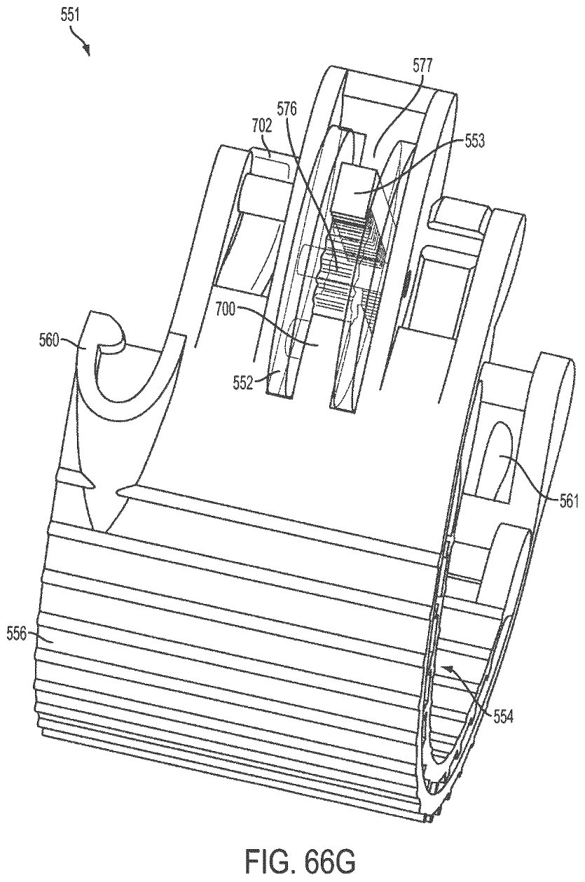

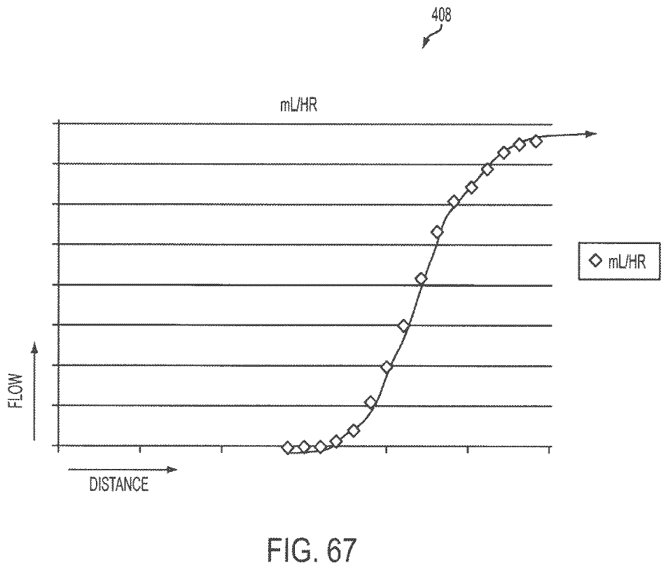

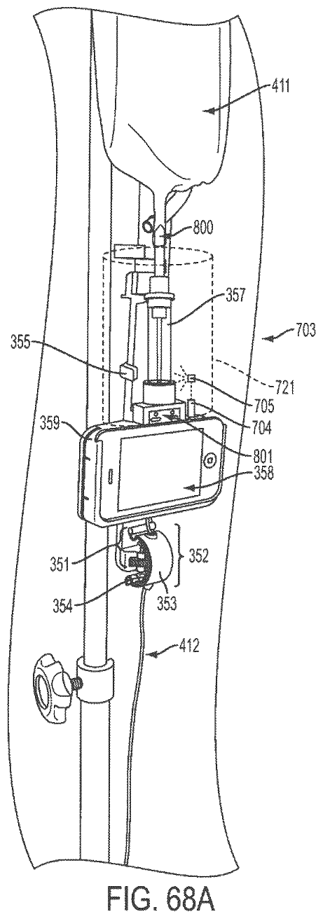

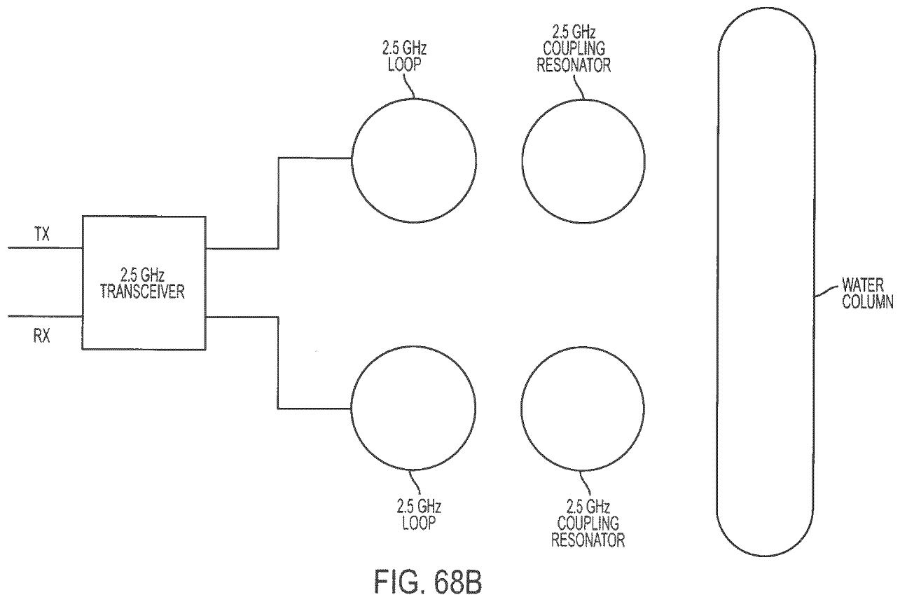

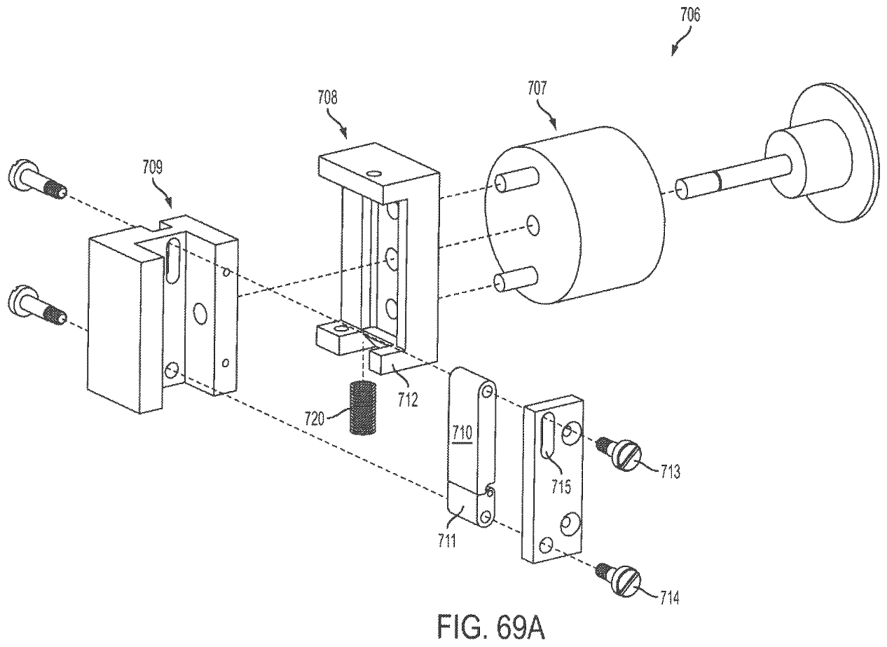

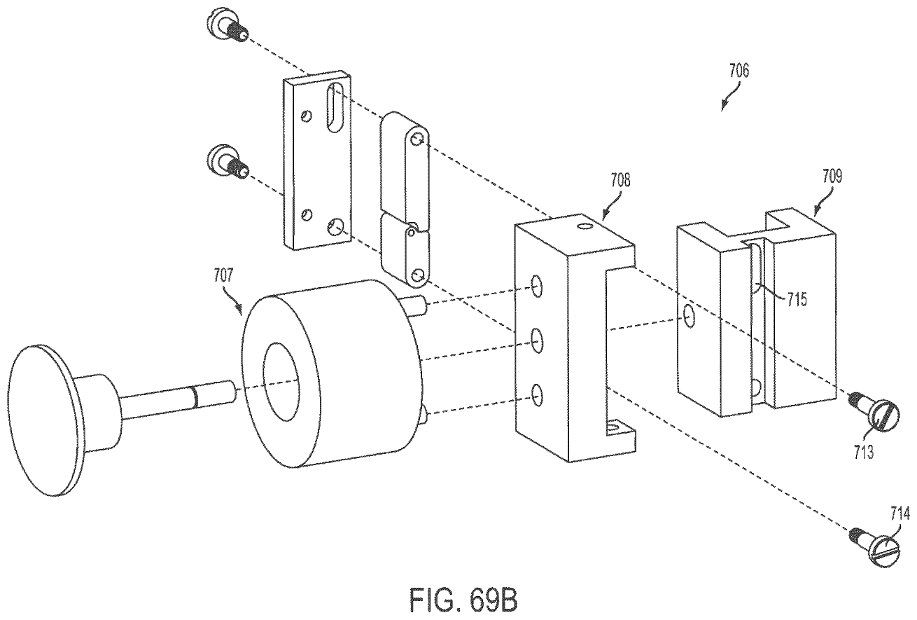

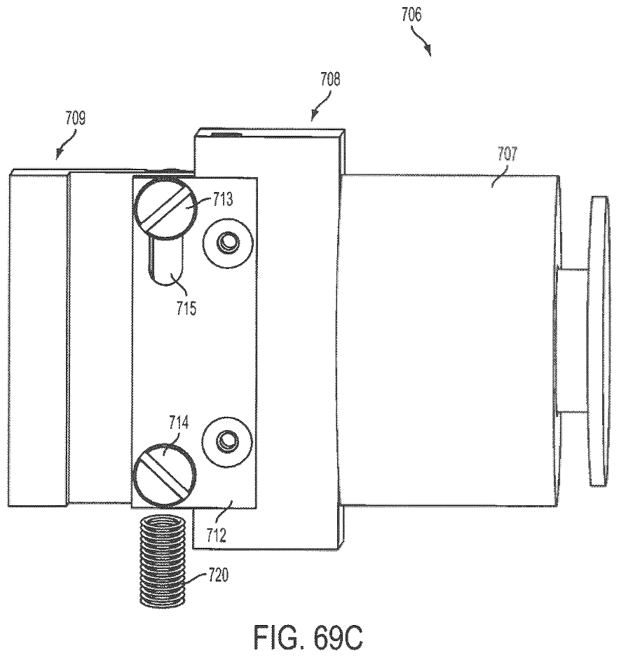

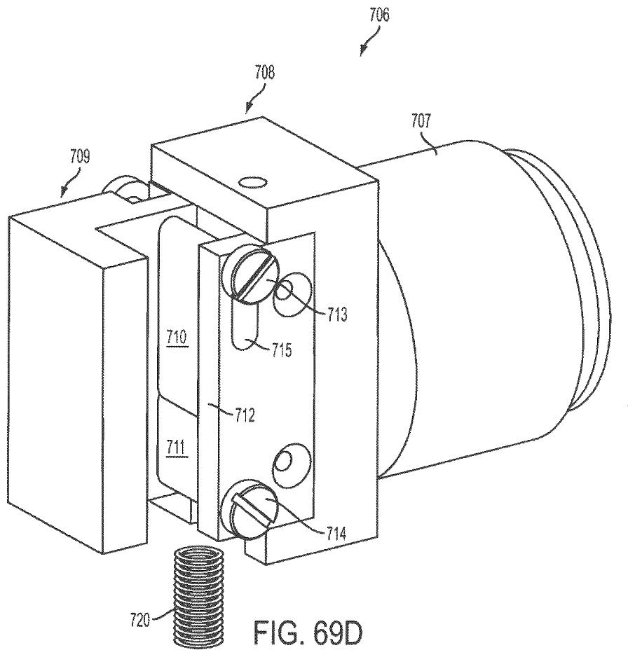

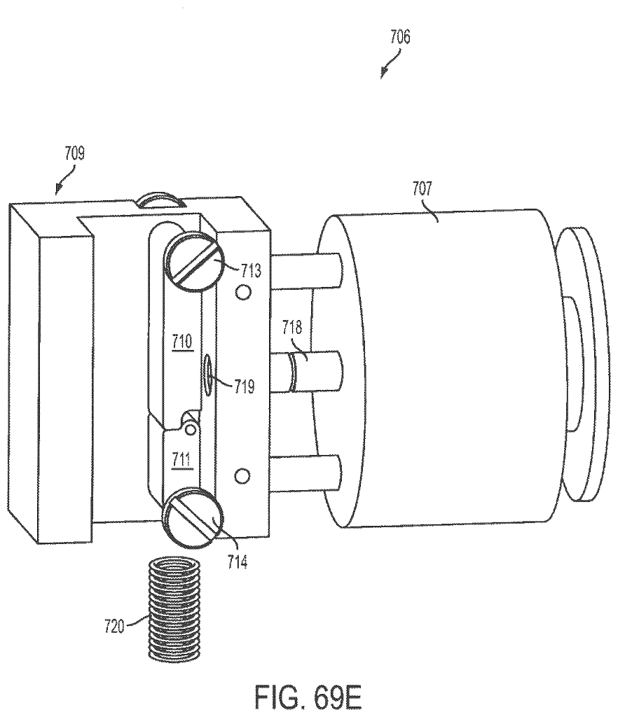

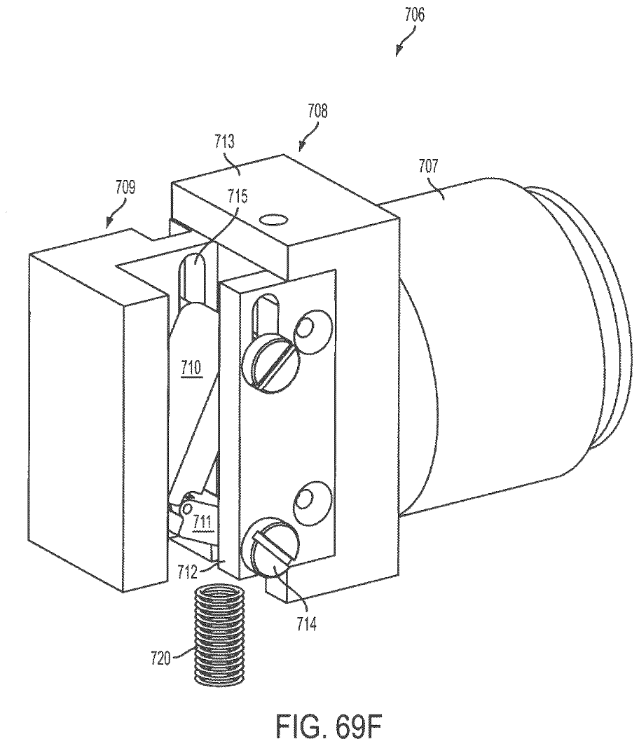

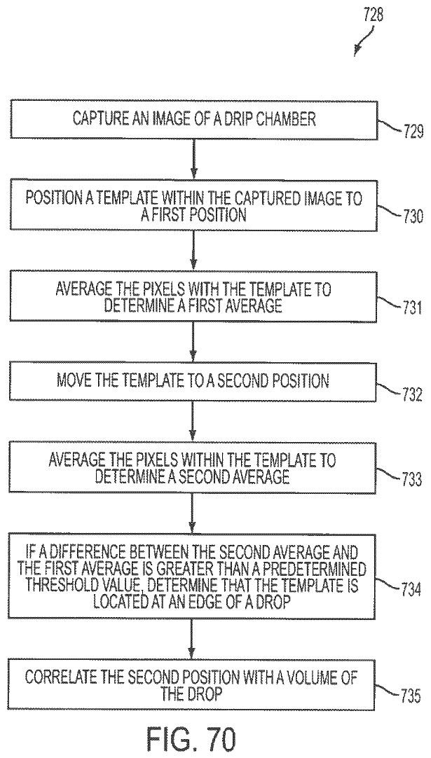

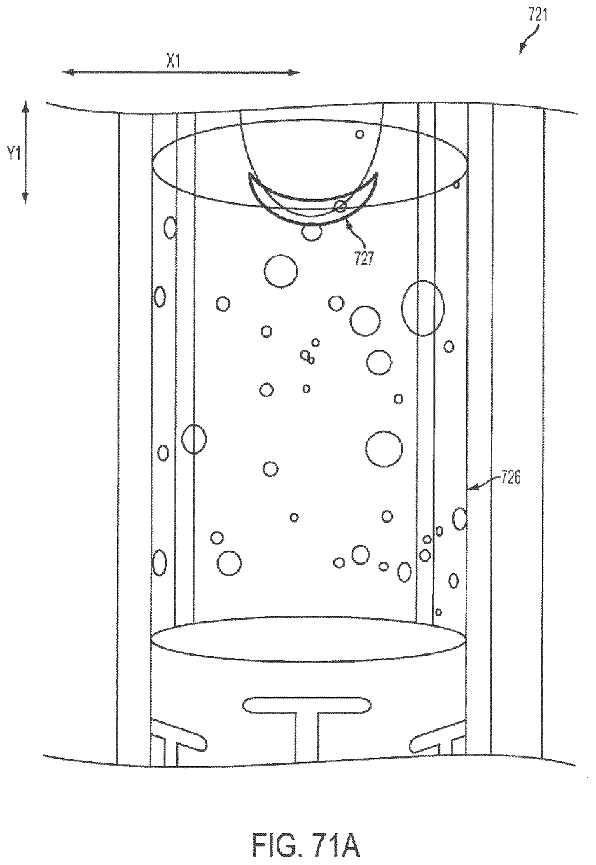

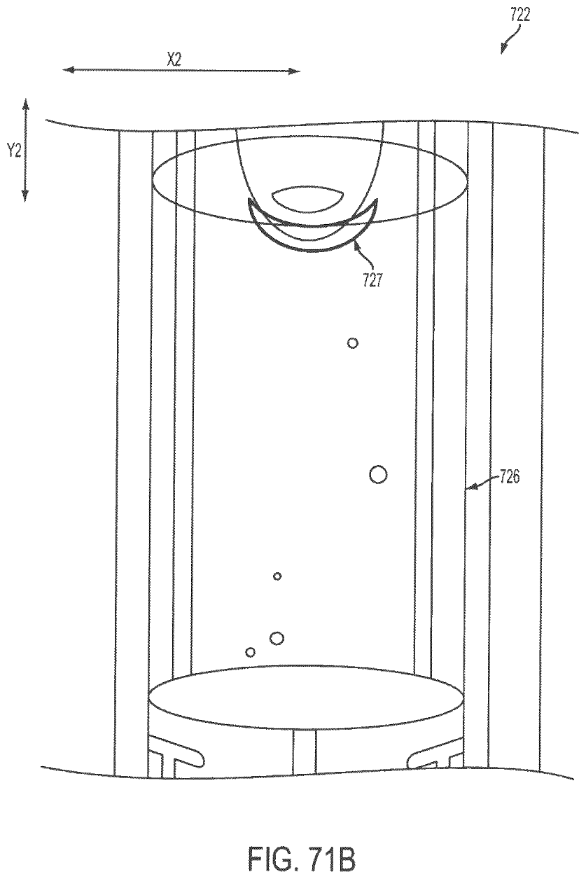

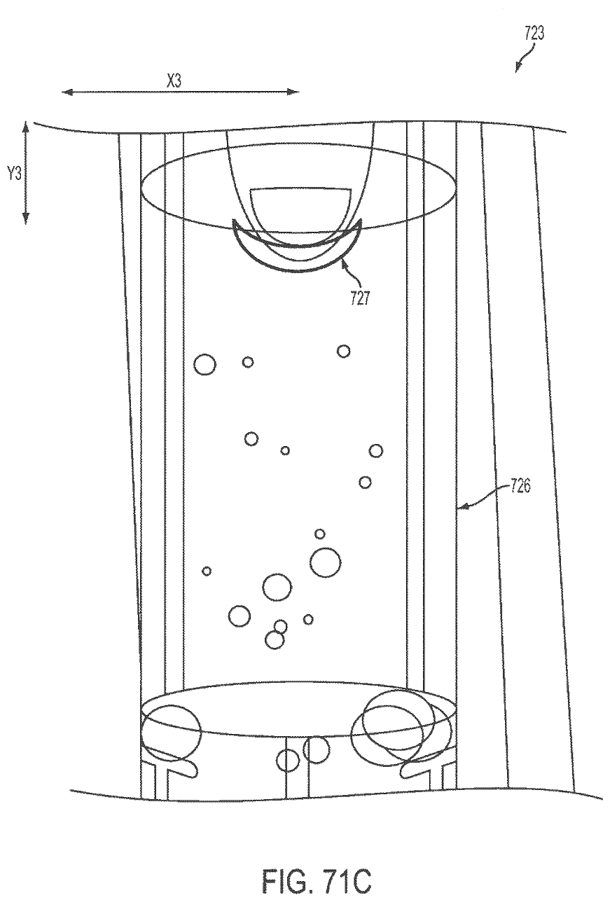

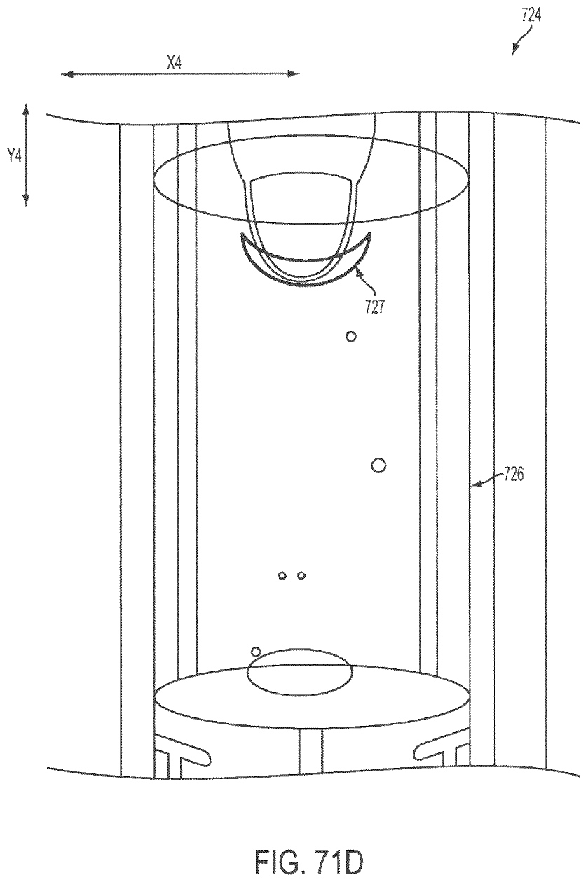

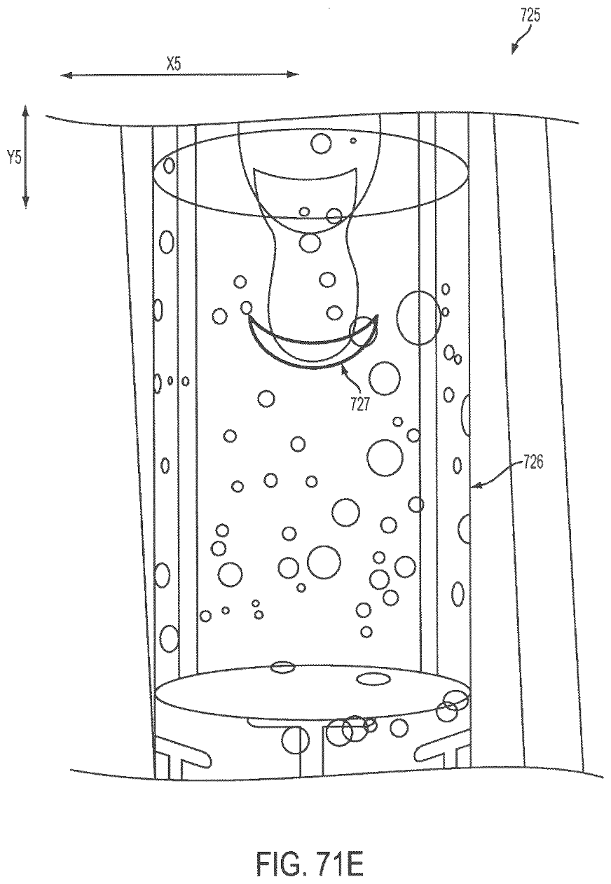

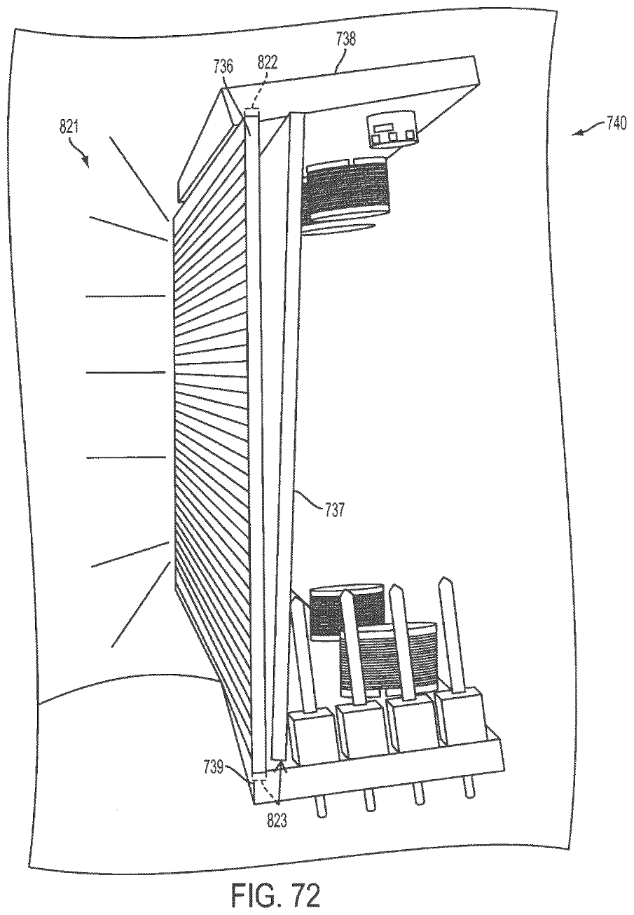

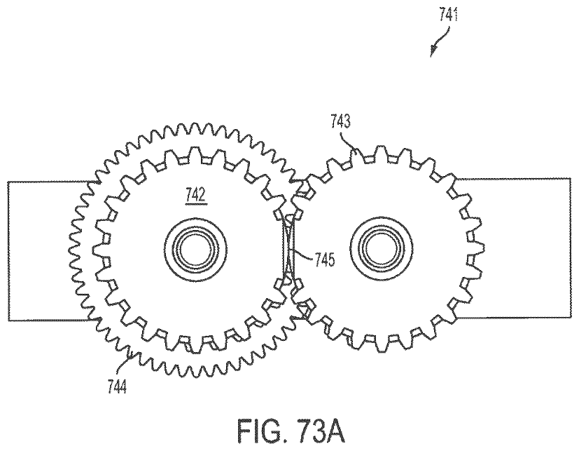

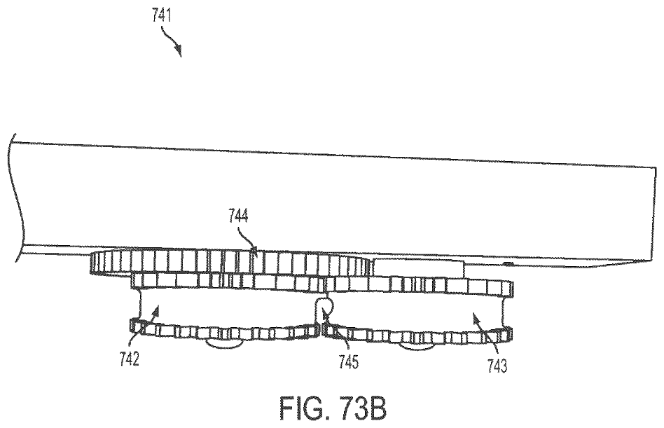

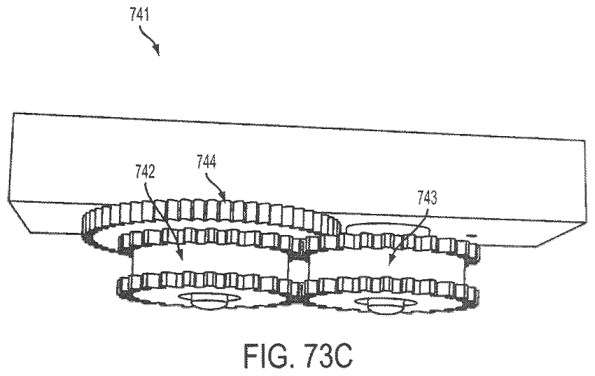

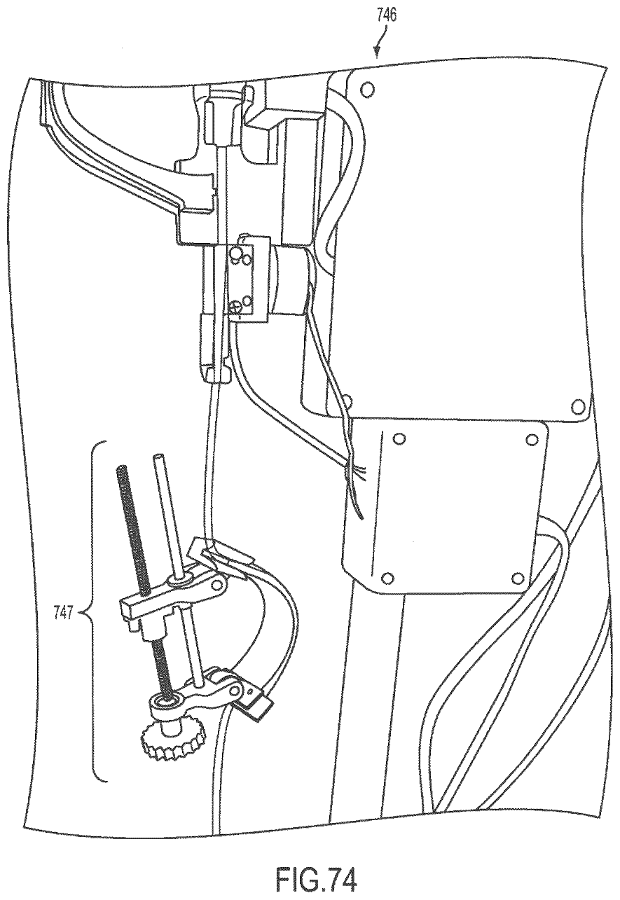

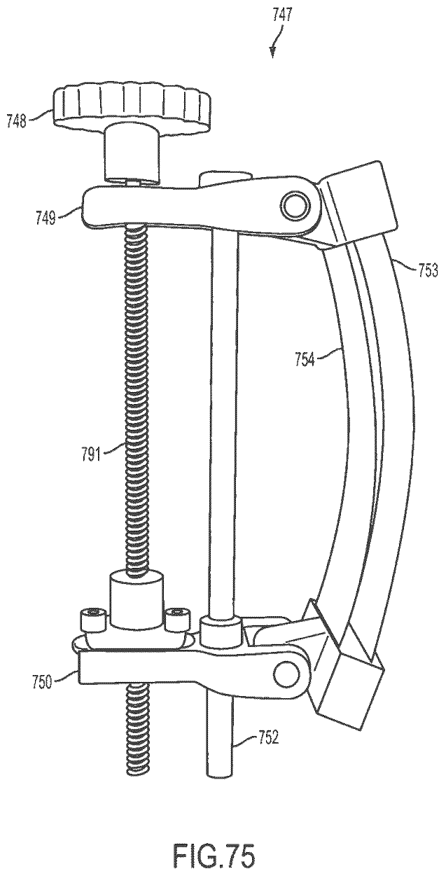

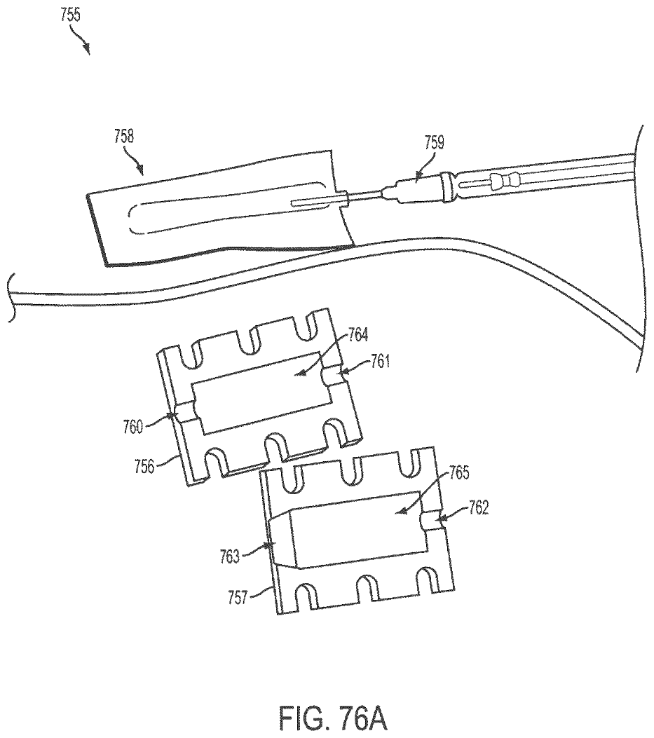

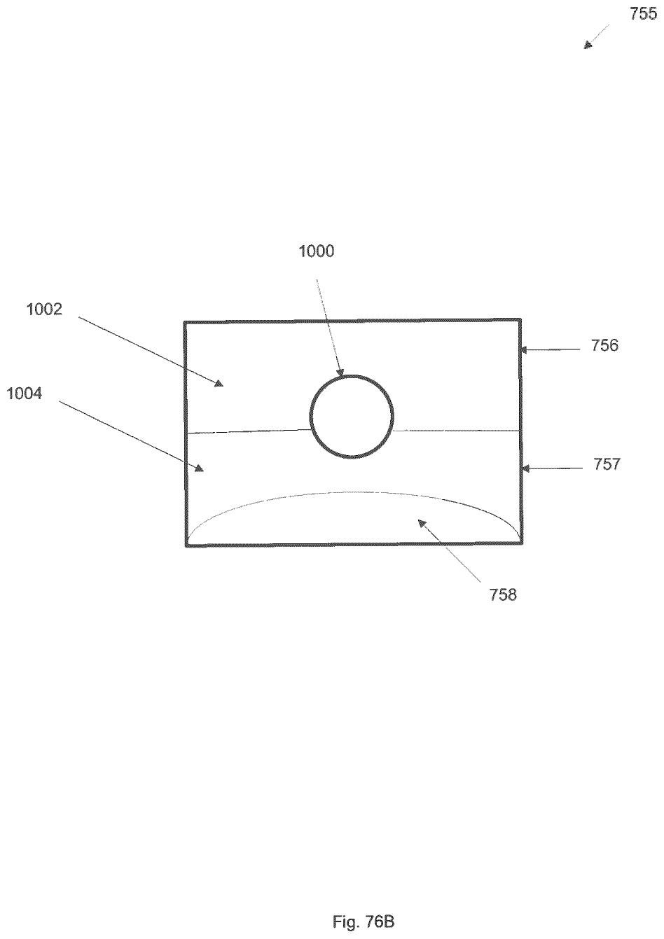

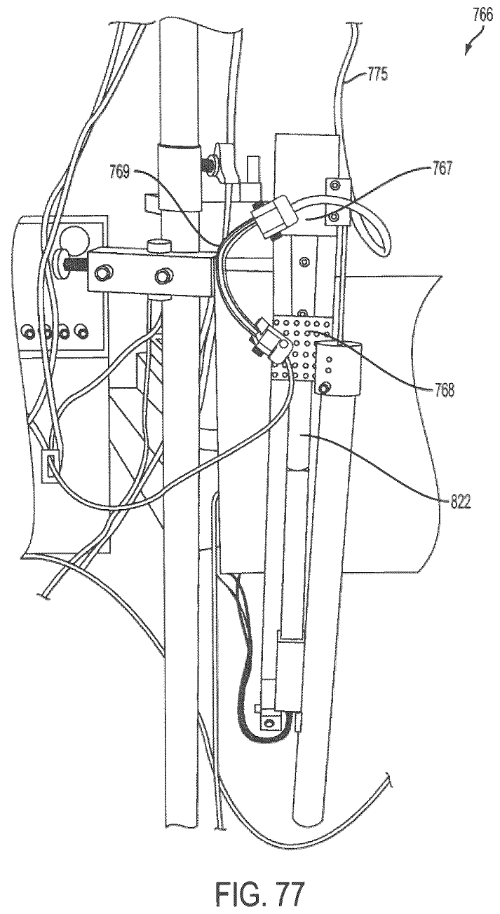

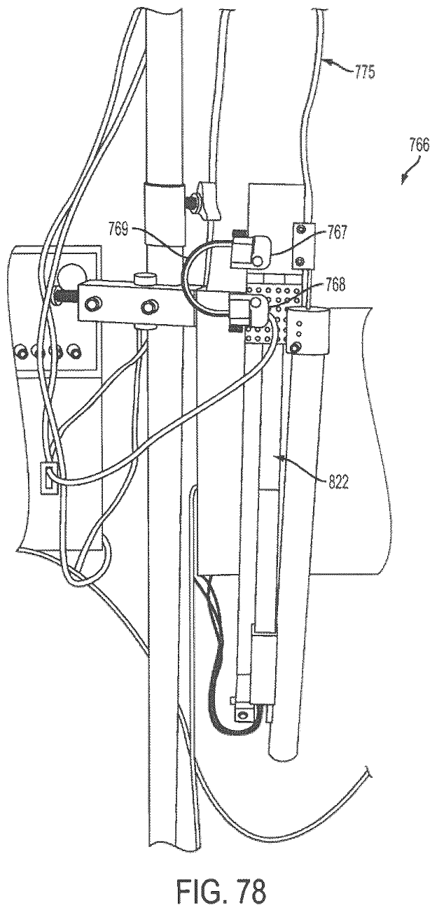

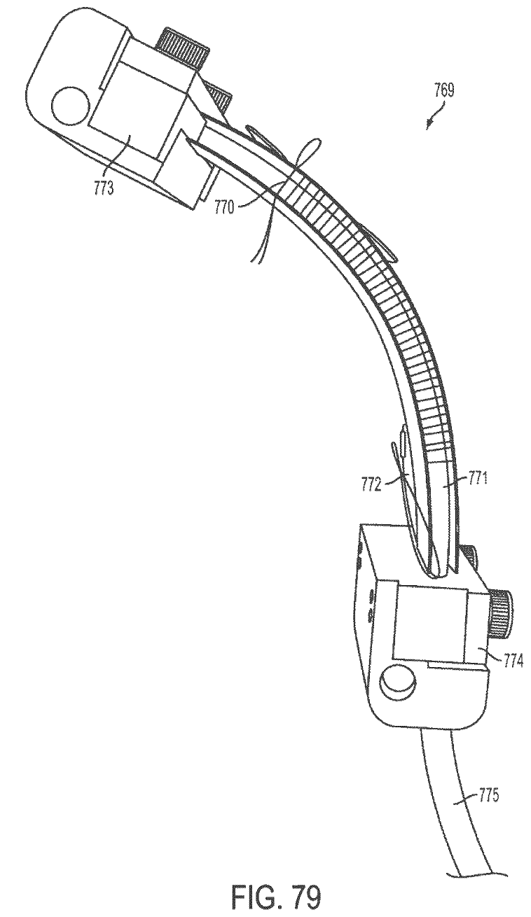

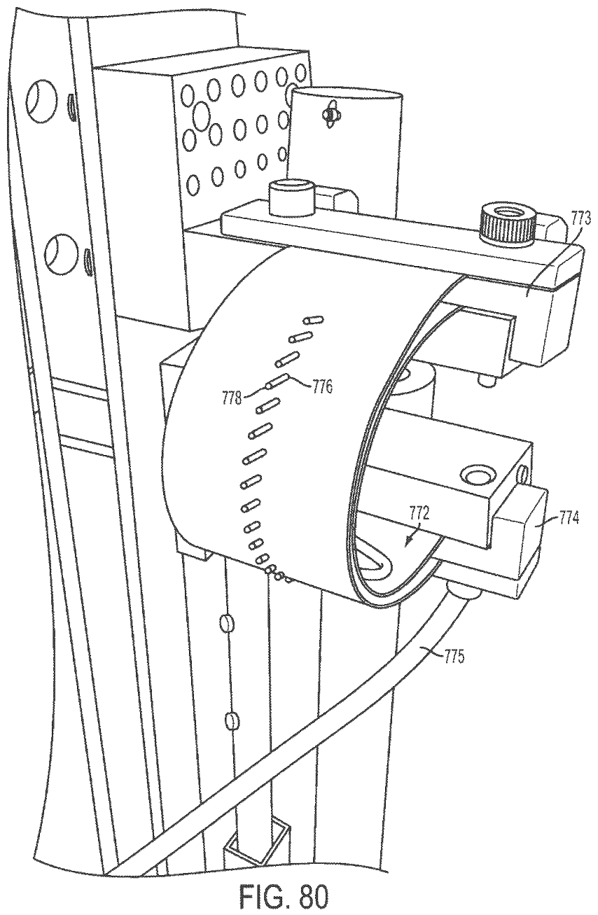

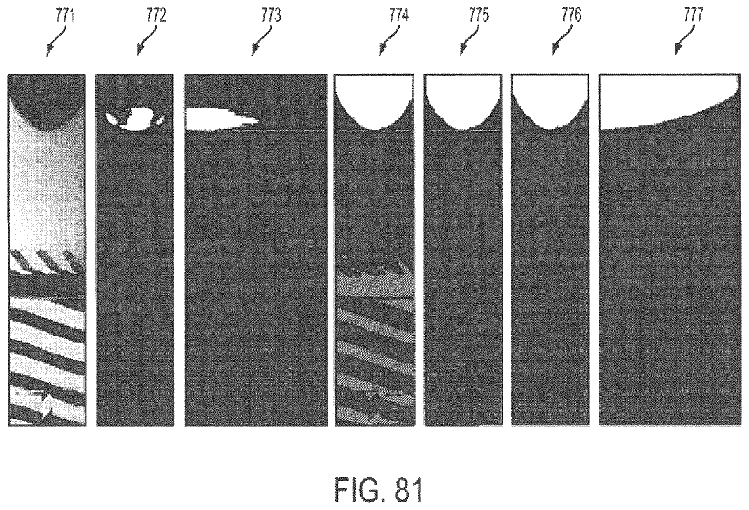

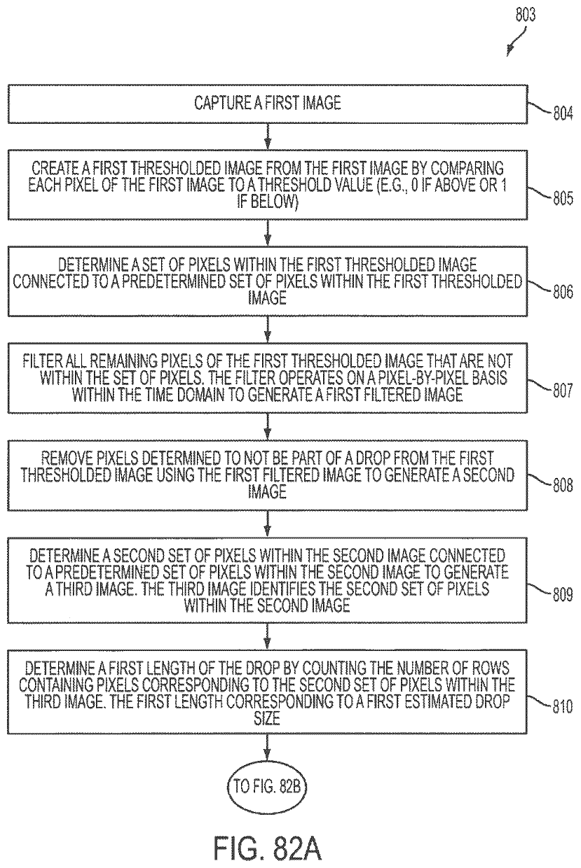

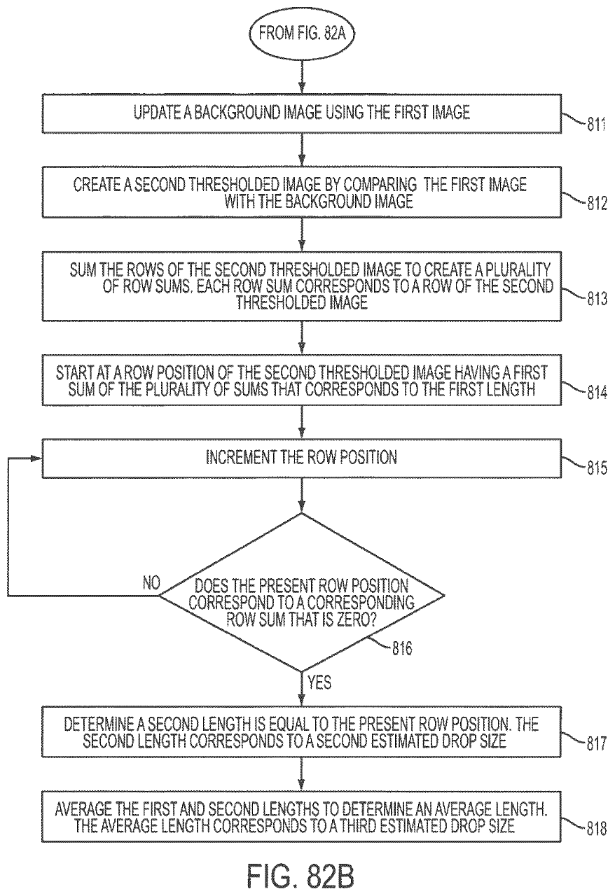

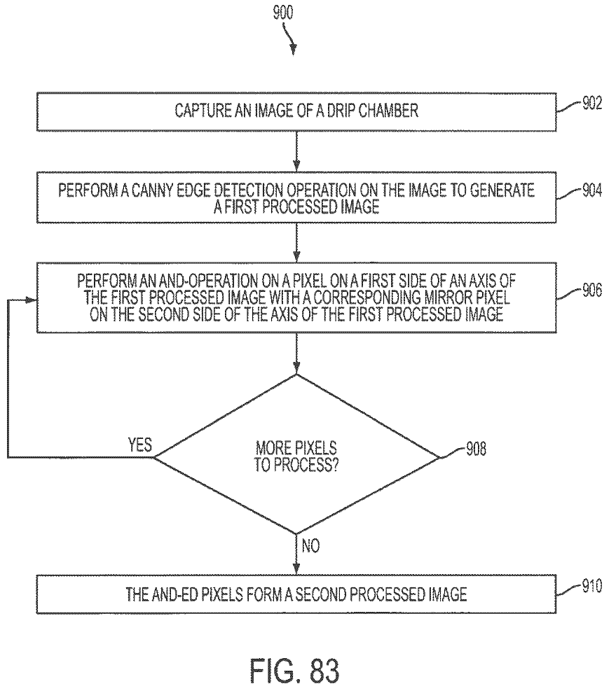

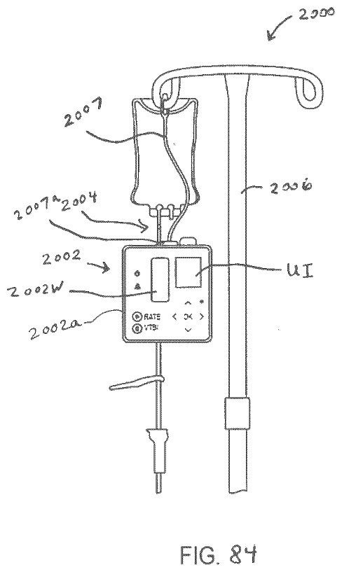

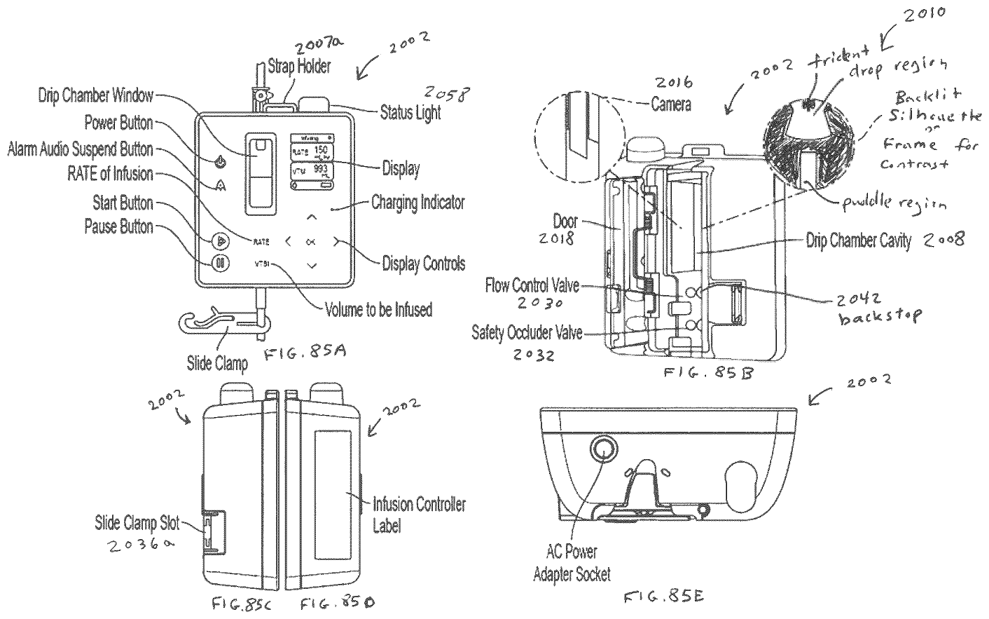

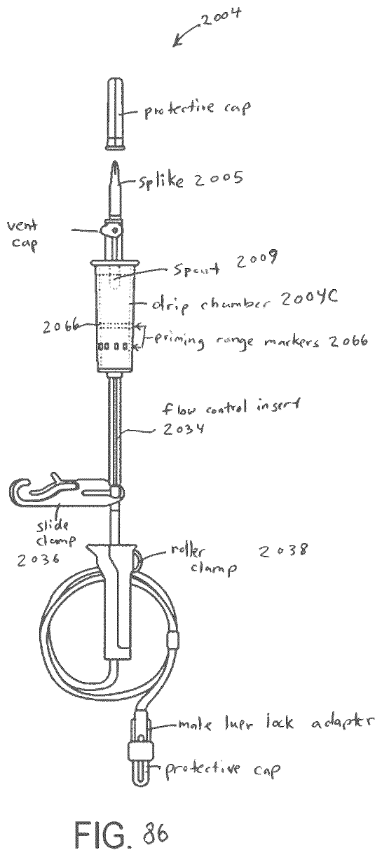

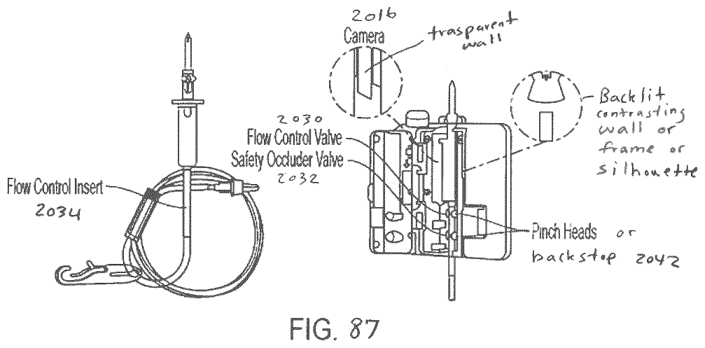

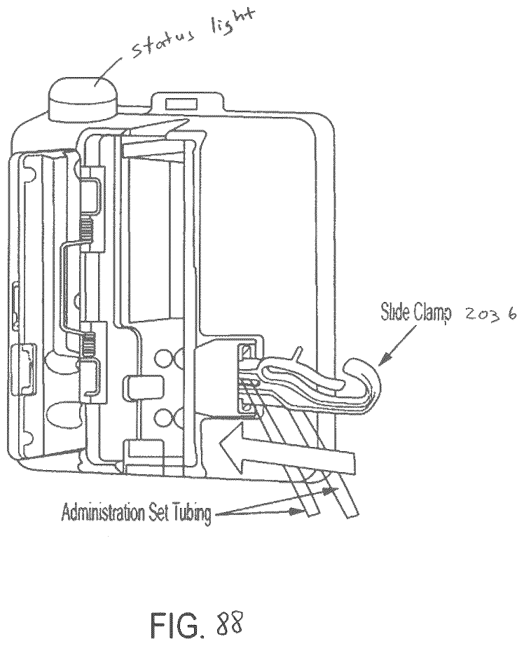

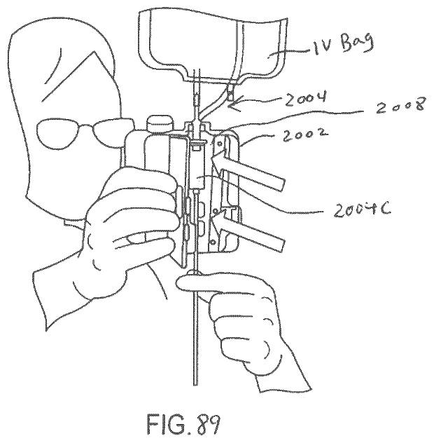

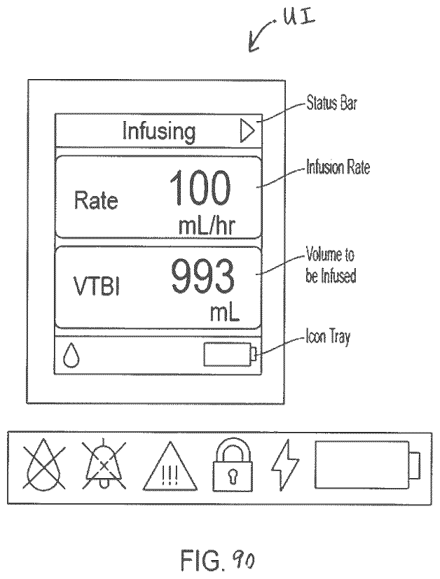

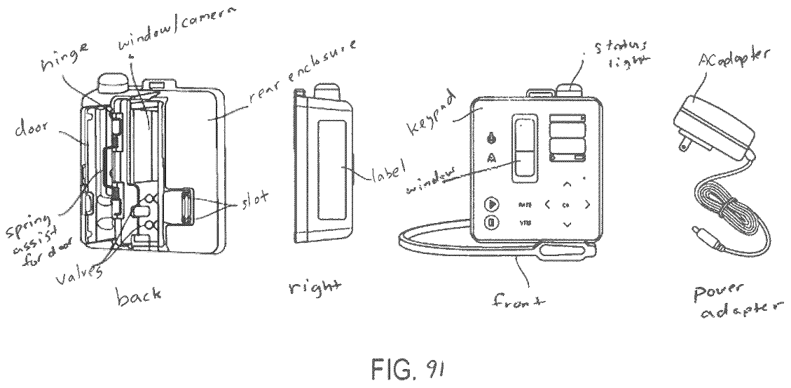

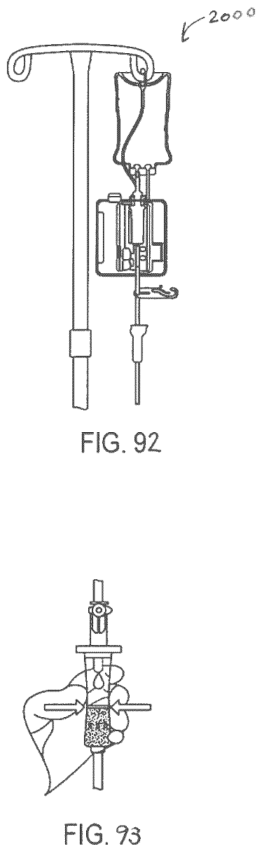

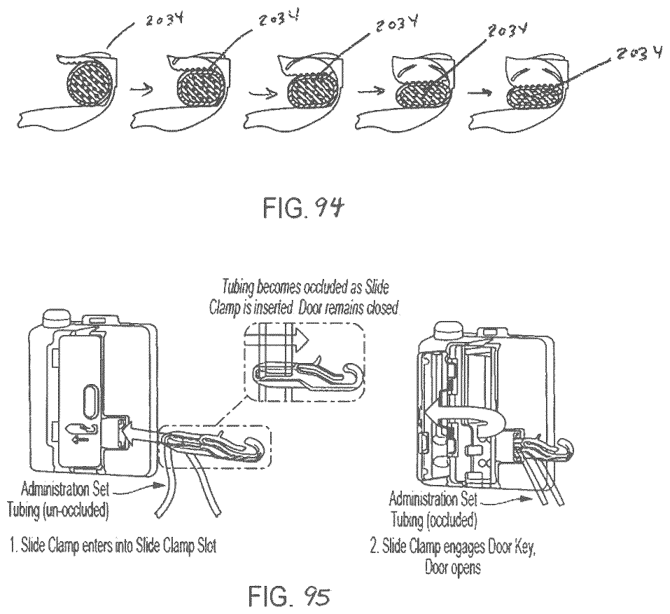

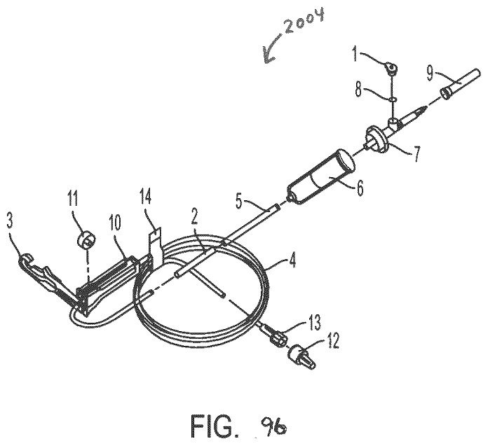

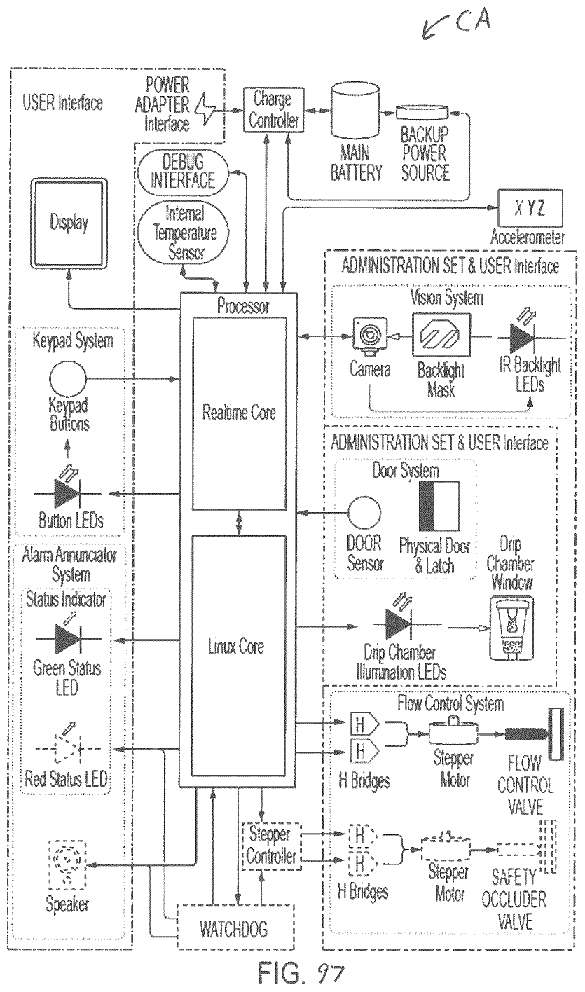

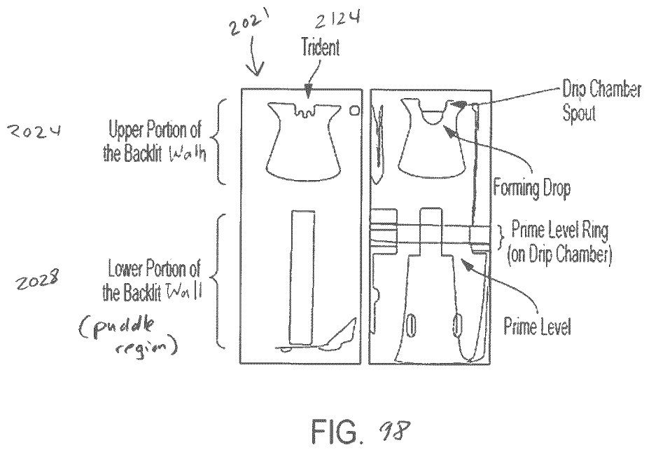

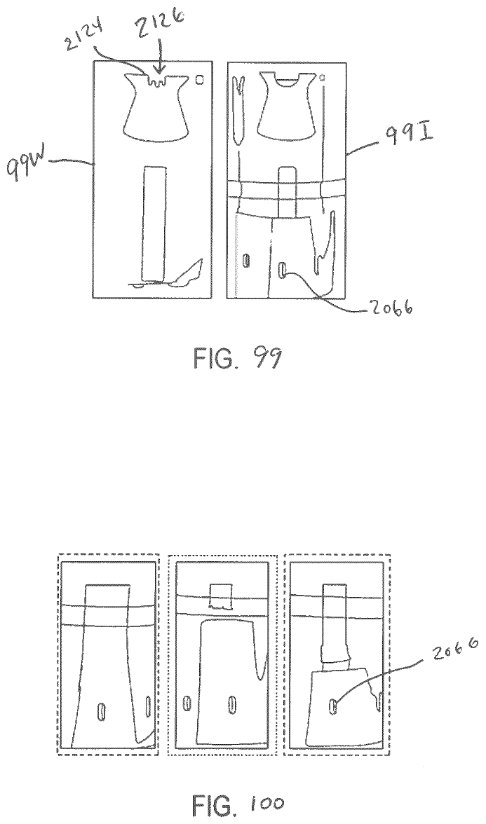

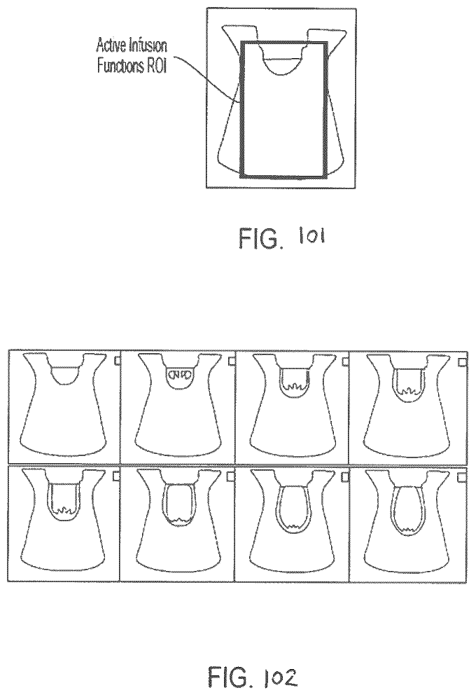

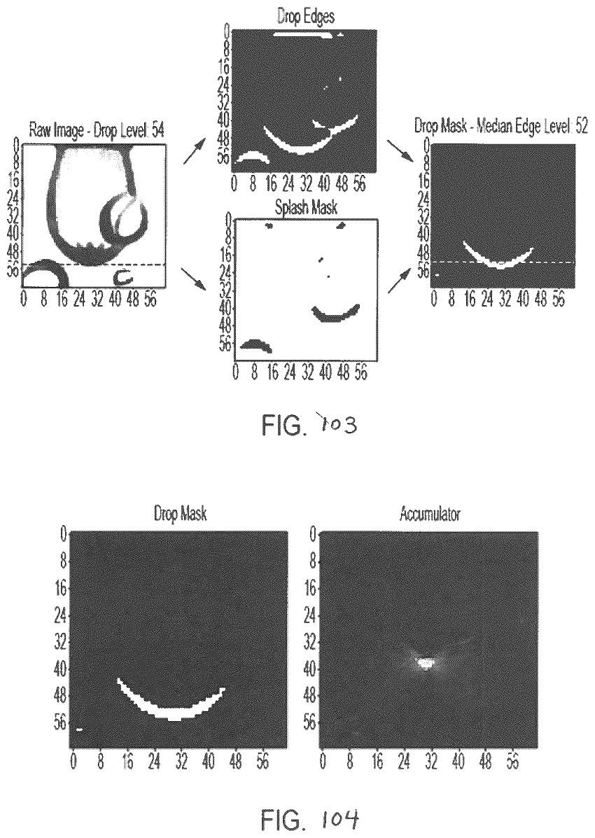

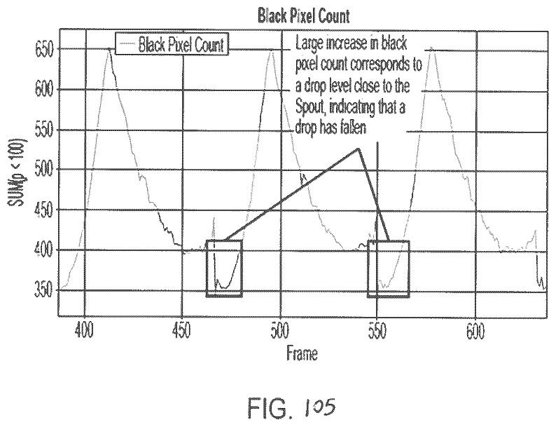

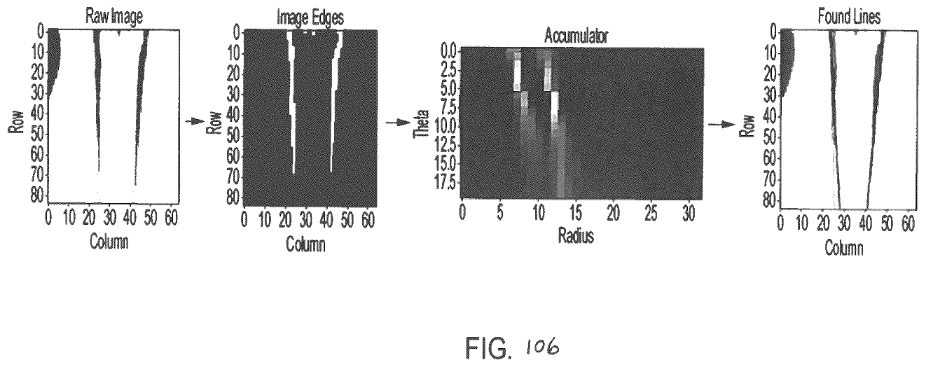

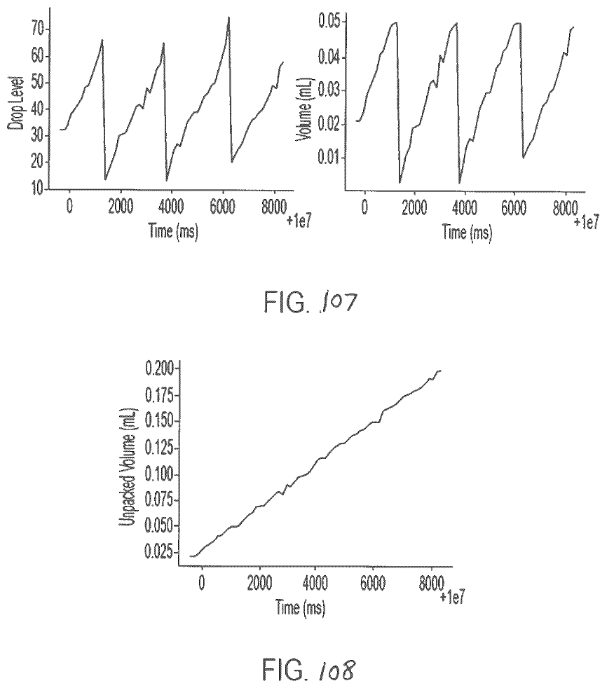

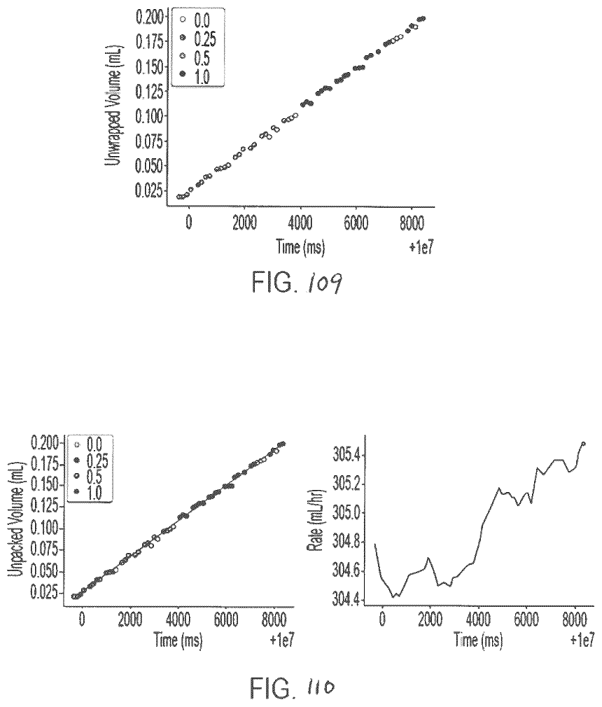

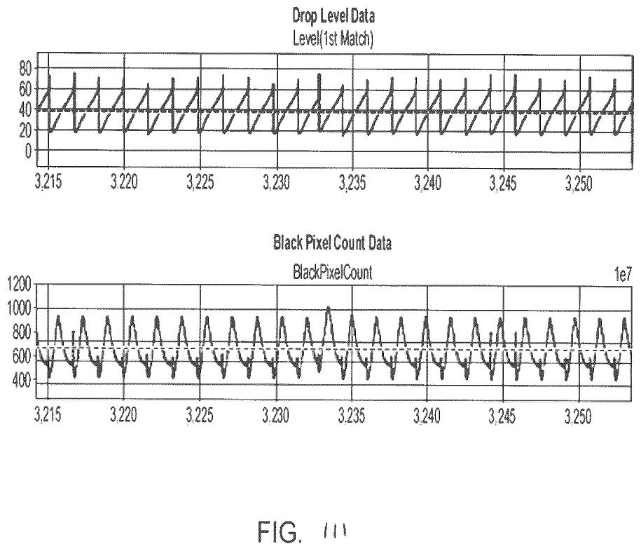

FIG. 64A shows a valve 542 having a slidable collar 545 and several compressing fingers 544 for regulating fluid flow through a fluid line 543 in accordance with an embodiment of the present disclosure. The base 546 is connected to all of the fingers 544. As the slidable collar 545 is moved over the compressing fingers 544, the compressing fingers 544 compress the tube 543 to impede fluid flow therewithin.

The fingers 544 are coupled to a base 546 such that the base 546 and fingers 544 surround the tube 543. The collar 545 is slidable away from the base 546 such that the fingers 544 compress the tube 543 which thereby reduces an internal volume of the tube 543. The reduction of the internal volume of the tube 543 reduces the fluid flow through the tube. An actuator (not shown) may be coupled to the collar 545 to control the position of the collar 545 (e.g., a linear actuator may be coupled to the collar 545 and to the base 546). FIG. 64B shows a cross-sectional view of the valve 542 of FIG. 64A. Note that the fingers 544 may be shaped away from the tube near an opposite end of the base

FIG. 65 shows a valve 547 having two curved surfaces 549 and 550 for positioning a fluid tube 548 therebetween to regulate fluid flow through the fluid tube 548 in accordance with an embodiment of the present disclosure. As the surfaces 549, 550 are compressed together, the tube 548 is compressed therebetween. The two curved surfaces 549 and 550 may be compressed together using an actuator. The tube 548 may be wrapped several times around the surface 549.

FIGS. 66A-66G show several views of a valve 551 having a knob 552 to move a connecting member 553, which is locked into position after movement of the knob 552, in accordance with an embodiment of the present disclosure.

The valve 551 includes an inner curved, elongated support member 554 and an outer curved, elongated support member 556. A knob 552 is pivotally coupled to the outer support member 556 via a pin 578. A connecting member 553 engages teeth 576 of the knob 552.

The connecting member 553 may be inserted into a hole of an end 555 of the support member 556 such that rotation of the knob 552 frictionally locks an engaging finger 700 (see FIG. 66G) into the gear rack 558 of the connecting member 553. The engaging finger 700 may engage the teeth 576 to lock the knob 552 to thereby prevent rotation of the knob 552 unless sufficient torque overcomes the locking action of the engaging finger 700. A retaining finger 577 is positioned on the other side of the hole 571 to press the connecting member 552 against the teeth 576 of the knob 552.

The inner support member 554 can pivot out away from the outer support member 556 such that a tube can be loaded via raised portions 559 and 560 (see FIG. 66C). The inner support member 554 pivots away from the outer support member 556 via dog bone linkers 561, 562, 701, and 702 as shown in FIG. 66C. Thereafter, the inner support member 554 pivots back towards the support member 556 as shown in FIG. 66D. The connecting member 553 is then inserted into an end 555 of the outer support member 556 (a close up of the insertion is shown in FIG. 66E) that includes the engaging finger 700 that locks onto the teeth 576 of the knob 552 which temporarily immobilizes the connecting member 553 (see FIG. 660). The other end 581 of the connecting member 553 is locked into a hole 582 of an end 557 of the support member 556. The connecting member 553 may be pivotally connected to the end 557. The knob 552 includes teeth 576 to move the connecting member 553 in or out of the end 555. However, when the knob 552 is not moved, the engaging finger 700 locks the movement of the knob 552 unless a predetermined amount of torque clicks the finger 700 to the next tooth of the teeth 576 of the inner portion of the knob 552.

As previously mentioned, the support member 554 can swing away from the outer support member 556 as is shown in FIG. 66C, which is facilitated by the dog bone linkers 561, 562, 701, and 702. The dog bone linker 561 includes a pivot hole 572 that couples to a pivot 563 and a pivot hole 573 that couples to a pivot 565. The dog bone linker 562 includes a pivot hole 575 that couples to a pivot 566 and a pivot hole 574 that coupled to a pivot 566. The dog bone linker 701 couples to pivots 567 and 570, and the dog bone linker 702 couples to pivots 568 and 569 so that the end of the support member 556 also swings away from the inner support member 554.

FIG. 67 shows a graphic 408 that illustrates actuation vs. flow rates for a valve in accordance with an embodiment of the present disclosure. The graphic 408 shows the operation of a valve having elongated support members, such as, for example, the valve 340 of FIGS. 49 and 50A-50B, the valve 352 of FIGS. 51A-54, the valve 369 of FIG. 55, the valve 380 of FIGS. 56A-56C, the valve 380 of FIGS. 57A-57E, the valve 401 of FIGS. 58A-58D, the valve 500 of FIG. 59H, the valve 520 of FIGS. 60-60, the valve 542 of FIGS. 64A-64B, the valve 547 of FIG. 65, and/or the valve 551 of FIGS. 66A-66G. The x-axis of the graphic 408 shows the displacement between the ends of the support members of the valve, and the y-axis shows the flow rate (e.g., caused by gravity and/or a pressure source). The response of the valve is a nonlinear function, such as an S-curve, a sigmoid curve, a Gompertz curve, or a generalized logistic function. These functions may be adjusted to match the valve and/or the valve may be adjusted to match one of the curves or functions.

FIG. 68A shows a flow meter 703 that uses binary optics 705 in accordance with an embodiment of the present disclosure. The flow meter 703 includes a camera 355 that captures one or more images to estimate a flow rate of fluid through a drip chamber 357 using any sufficient method, e.g., the methods disclosed herein. The flow meter 703 includes a laser 704 that directs a laser beam onto a binary-optics assembly 705. The binary-optics assembly 705 thereafter redirects and reforms the laser beam through the drip chamber 357 and onto the image sensor 355 such that the image sensor 355 sees a pattern, e.g., the array of lines 85 shown in FIG. 8 which may form stripes as shown in the background pattern 89 of FIG. 10. The binary-optics assembly 705 may form the stripes by using a plurality of ovals.

The image sensor 355 may include a filter to filter out all frequencies except for the frequency of the laser 704. For example, the image sensor 355 may include an optical, band-pass filter that has a center frequency equal to (or about equal to) the optical frequency (or center frequency of the optical frequency) of the laser 704.

The monitoring client 358 may be electrically coupled to the laser 704 to modulate the laser 704. For example, the monitoring client 358 may turn on the laser 704 only when predetermined pixels are being exposed and may turn off the laser 704 when other pixels besides the predetermined pixels are being exposed.

The flow meter 703 optionally includes a first electrode 800 and a second electrode 801. The monitoring client 358 may be electrically coupled to the first and second electrodes 800, 801 to measure a capacitance defined therebetween. In streaming conditions, the capacitance changes because the relative permittivity is different for air and water. The monitoring client 358 may monitor the changes that results from a streaming condition with the drip chamber 357 by monitoring the capacitance between the first and second electrodes 800, 801 and correlate increases and/or decreases of the capacitance beyond a threshold as corresponding to either a streaming condition and/or a non-streaming condition. For example, if the capacitance between the first and second electrodes 800, 801 is higher than a threshold, a processer within the monitoring client 358 may determine that the drip chamber 357 is undergoing a streaming condition.

In an alternative embodiment, the first and second electrodes 800, 801 are loop antennas. The monitoring client 358 uses a transceiver to monitor the magnetic coupling between the loop antennas 800, 801. For example, the transceiver may transmit a coded message from one loop antenna of the antennas 800, 801, to another one of the loop antennas 800, 801 and then determine if the coded message was successfully received. If so, then a received signal strength indication (“RSSI”) measurement may be made from the transceiver. See FIG. 68B for an exemplary circuit. The RSSI may be used to monitor the magnetic coupling between the antennas 800, 801. If the magnetic coupling is above a threshold, then the monitoring client 358 may determine that a streaming condition exists within the drip chamber 357. In some embodiments a change of magnetic coupling or a change of capacitive coupling may be determined to be an indication that a streaming condition has occurred.

The flow meter 703 may also include a safety valve 706. FIGS. 69A-69F show several views of the safety valve 706 that may be used with a flow meter, such as the flow meter 703 of FIG. 68, in accordance with an embodiment of the present disclosure.

FIGS. 69A-69B show exploded views of the safety valve 706. The safety valve 706 includes a solenoid 707, an interface structure 708, a tube housing 709, a spring 720, a faceplate 712, a first axle 713, a second axle 714, a first occluding arm 710, and a second occluding arm 711. The faceplate 712 includes a hole 715, and the tube housing 709 also includes a hole 819. The holes 715, 819 allow the axle 713 to slide within the holes 715, 819.

As shown in FIG. 69C, a tube 820 may be placed within the tube housing 709 which places the tube 820 next to the first and second occluding arms 710, 711, which are easily seen in FIG. 69D. A spring 720 keeps the first and second occluding arms 710, 711 retraced when in the retracted state (as shown in FIG. 69D), but stores energy such that a predetermined amount of movement of the first and second occluding arms 710, 711 towards the tube 810 cause the spring 720 to discharge its stored mechanical energy to cause the first and second occluding arms 710, 711 to extend out and occlude the tube 820. The spring 720, which may be a compression spring 720, may pull the first and second axles 713, 714 towards each other. The first and second occluding arms 710, 711 are pivotally connected together. As is easily seen in FIG. 69E, a shaft 718 of a solenoid 707 can actuate through a hole 719 to push on the spring 720 which causes the spring 720 to release its energy and occlude the tube 820 (see FIG. 69F for the case when the where the first and second occluding arms 710, 711 are in the occluding position).

FIG. 70 shows a flow chart diagram illustrating a method 728 of estimating drop growth and/or flow within a drip chamber in accordance with an embodiment of the present disclosure. The method 728 includes acts 729-735. FIGS. 71A-71E show images taken by a flow meter with a template overlaid therein to illustrate the method of FIG. 70.

Act 729 captures an image of a drip chamber. The image captured may be the image 721 of FIG. 71A. Act 730 positions a template within the captured image to a first position. For example, as shown in FIG. 71A, a template 727 may be positioned within a predetermined position. Act 731 averages all of the pixels within the template 727. Act 732 moves the template to a second position. For example, the template 727 in FIG. 71A may move the template in the Y direction (e.g., down as seen in FIG. 71A).

In act 733, the pixels within the template are used to determine a second average. In act 734, if a difference between the second average and the first average is greater than a predetermined threshold value, determine that the template is located at an edge of a drop. For example, referring to FIG. 71A, the template may be slowly lowered down in the Y direction, until the template 727 transitions from the edge of a drop to a portion of the image that doesn't contain the drop, in which case the average value of the pixels will transition abruptly to a dark average to a lighter average. When this transition occurs, the Y position of the template 727 is considered to be at the edge of the drop (e.g., Y1 of FIG. 71A). In act 735, the second position of the drop is correlated with a volume of the drop. For example, the Y1 value may be associated with a volume of a drop in a lookup table. In some embodiments of the present disclosure, multiple movements of the template 727 are needed to until the edge of the drop is detected. For example, the template 727 may be moved in the y-direction one pixel at a time (or several pixels at a time) and several template 727 movements may be needed such that the edge of the drop is detected. By monitoring the edge of the drop, the growth of the drop may be controlled by the flow meter to achieve a target flow rate (e.g., the rate of the transition between Y1 of FIG. 71A to Y2 of FIG. 71B may be controlled by a PID control loop within a flow meter). FIG. 71B shows a location, Y2, that corresponds to a growth in the drop relative to the location, Y1, of FIG. 71A.

FIG. 72 shows a modulatable backlight assembly 740 in accordance with an embodiment of the present disclosure. The assembly 740 may be the backlight 18 of FIG. 1 or may be used as a backlight for any sufficient flow meter disclosed herein. The assembly 740 includes a first circuit board 738, a second circuit board 739, a first backlight diffuser 736, and a second backlight diffuser 737.

The first circuit board 738 includes embedded light sources 822 that extend along the interface between the first backlight diffuser 736 and the first circuit board 738. The embedded light sources 822 shine light into the first backlight diffuser 736 which is directed outwards as indicated by 821. The light 821 may be directed towards an image sensor. The first backlight diffuser 736 only diffuses light with no “pattern” formed when viewed by an image sensor.

The second circuit board 739 includes embedded lights 823 which are shined into the second backlight diffuser 737. The second backlight diffuser 737 creates a pattern of stripes that shows up in the light 821 when viewed by an image sensor. Therefore, a monitoring client (e.g., the monitoring client 358 of FIG. 51A) and/or a flow meter (e.g., the flow meter 7 of FIG. 1) can select between a striped background pattern (by activating the embedded lights 823) and a non-striped background pattern (by activating the embedded lights 822).

For example, referring now to FIGS. 1 and 72, the flow meter 7 may use the backlight assembly 740 in some specific embodiments; The flow meter 7 may use a non-striped backlight pattern (by activating the embedded LEDs 822 without activating the embedded LEDs 823) to monitor the growth of drops and may switch to a striped background pattern (by activating the embedded LEDs 823 without activating the embedded LEDs 822) to detect streaming conditions.

FIGS. 73A-73C show several views of a tube-restoring apparatus 741 in accordance with an embodiment of the present disclosure. The apparatus 741 includes a drive gear 744 that is coupled to a first restoring gear 742. The first restoring gear 742 is mechanically coupled to a second restoring gear 743. A tube may be placed between the first and second restoring gears 742, 743. Portions of the first and second restoring gears 742, 743 define a space 745 in which a tube may be positioned. Rotation of the first and second restoring gears 742, 743 closes the distance between the space 745 when the tube is positioned between the first and second restoring gears 742, 743. The transition from a non-restoring position to a restoring position is shown in FIG. 73B to FIG. 73C. For example, a tube may be positioned such that an occluder presses against the tube from the bottom up (as shown in FIG. 73B). If the tube becomes distorted over time, a motor connected to the driving gear 744 rotates the gears 743 and 744, to press against the walls of the tube (as shown in FIG. 73C) to restore the tube by compressing on the wall portions of the tube that are expanded beyond a center axis of the tube such that the tube is distorted into an oval shape, for example.

FIG. 74 shows a system for regulating fluid flow 746 using a valve 747 having two flexible strips 753 and 754 (see FIG. 75); And FIG. 75 shows the valve 746 of FIG. 74 in accordance with an embodiment of the present disclosure. Optionally, a motor may be attached to the valve 746 for control by a flow meter in one embodiment.

As shown in FIG. 75, the valve 747 includes two flexible strips 753, 754 in which a tube may be disposed therebetween, a guiding shaft 752, two guidable members 749, 750, a screw 791, and a knob 748.

When the knob 748 is turned, the screw 791 rotates. Rotation of the screw 791 pulls the distal guiding member 750 toward the proximal guiding member 749 (because the distal guiding member 750 includes internal threads and the screw 791 spins freely within the proximal guiding member 749). The guide 752 guides the movement of the distal guiding member 750. The guide 752 is coupled to the proximal guiding member 749.

FIG. 76A shows a valve 755 that utilizes a fluid-based bladder 758 in accordance with an embodiment of the present disclosure. The valve 755 includes two clamshells 756, 757, a bladder 758, and a piston 759. The piston 759 may be any fluid source. The bladder 758 may be placed within a cavity 764 and a tube may be placed across the bladder 758 and positioned within the throughways 760 and 761. Thereafter, the clamshell 757 may be placed over the bladder 758 such that the cavity 765 is placed over the bladder 758. The two clamshells 756, 757 may then be ultrasonically welded together, temporarily compressed together, and/or sufficiently held together. Thereafter, an actuator (e.g., an actuator controlled by a flow meter disclosed herein) may be actuated to move fluid in and out of the bladder 758 via the piston 759.

FIG. 76B shows a cross-sectional view of the assembled valve 755 of FIG. 76A with two elastomeric fillers 1002, 1004 in accordance with an embodiment of the present disclosure. The elastomeric fillers 1002, 1004 help hold the tube 1000 into position and help restore the tube 1000 when the bladder 758 is deflated.

FIG. 77 shows a system 766 for regulating fluid flow using a valve 769 having two flexible strips 771, 772 (see FIG. 79) actuatable by a linear actuator 822 in accordance with an embodiment of the present disclosure. FIG. 78 shows the linear actuator 822 actuating the valve 769 to impeded fluid flow through a tube 775. The valve 769 is coupled to two couplers 767 and 768. The proximal coupler 768 moves with the linear actuator 822 while the distal coupler 767 is fixed relative to a non-moving end of the linear actuator 822.

FIG. 79 shows a close-up of the valve 769 of FIGS. 77-78. The valve 769 includes two strips 771, 772 (which may be metallic strips) in which the tube 775 may be disposed. The two strips 771, 772 of the valve 769 may be coupled to a first end structure 773 and a second end structure 774. The first end structure 773 may be coupled to the distal coupler 767 and the second end structure 774 may be coupled to the proximal coupler proximal coupler 768 (see FIGS. 77-78). A string 770 or membrane may be wrapped around the tube 775 such that, when the strips 771, 772 are straightened out, the string 770 presses against the side walls of the tube 775 to help round the tube 775. The membrane may be a flexible, but not stretchable, material (or minimally stretchable material). FIG. 80 shows a close-up of the valve as actuated in FIG. 78. Note the holes 776 and 778 that the string 770 is threaded through. The string 770 (which may metallic) is spiraled around the tube 775 such that when the valve 769 opens, the string 770 restores the tube 775.

FIG. 81 shows several images for use to illustrate a method of estimating drop growth and/or fluid flow illustrated in FIGS. 82A-82B in accordance with an embodiment of the present disclosure. FIG. 81 shows images 771-777 which are referred to below regarding FIGS. 82A-82B.

FIGS. 82A-82B show a flow chart diagram illustrating a method 803 of estimating drop growth and/or fluid flow. The method 803 includes acts 804-818.

Act 804 captures a first image (e.g., image 771 of FIG. 81). The first image may be a grey scale image of the drip chamber. The drip chamber may be uniformly lit with a striped pattern on the bottom of the chamber (i.e., there is no back pattern on the top portion of the drip chamber).

Act 805 creates a first thresholded image using the first image. The first thresholded image may be the image 774 of FIG. 81. The first thresholded image may be made by comparing each pixel from the first image to a threshold value (e.g., setting a respective pixel of the threshold image to 0 if the respective pixel of the first image is above the threshold or setting a respective pixel of the thresholded image to 1 if the respective pixel of the first image is below the threshold). This act is to highlight areas where there is water in front of the background.

In some specific embodiments, the threshold level is updated every time a new image is taken to ensure a predetermined ratio of 1 to 0 pixels is maintained to highlight the drop. The ratio may be updated for use by act 805 when used again or the update may adjust the threshold until a predetermined ratio of 1 to 0 pixels is made and then use the first thresholded image for the rest of the method 803.

Act 806 determines a set of pixels within the first thresholded image connected to a predetermined set of pixels within the first thresholded image. The predetermined set of pixels may be determined by fiducials marked on the drip chamber or an opening in which drops are formed. The predetermined set of pixels may be a predetermined set of x, y values that correspond to pixels. Act 806 may use a connected component image analysis algorithm.

Act 807 filters all remaining pixels of the first thresholded image that are not within the set of pixels. The filter operates on a pixel-by-pixel basis within the time domain to generate a first filtered image. The first filtered image is an estimate of a non-active (e.g., a result from features not of interest in the image) portion of the first thresholded image (image 774 of FIG. 81). The filter may be any filter, e.g., any filter described herein.

Act 808 removes pixels determined to not be part of a drop from the first thresholded image using the first filtered image to generate a second image (e.g., image 775 of FIG. 81). A pixel within the second image will be set to 1 if a respective pixel in the first thresholded image is 1 and a respective pixel in the first filtered image is less than 0.5; otherwise, the pixel will be set to 0.

Act 809 determines a second set of pixels within the second image connected to a predetermined set of pixels within the second image to generate a third image (e.g., the image 776 of FIG. 81). The third image identifies the second set of pixels within the second image. Act 809 finds the set of “lit” pixels in the second image connected to the predetermined set of pixels (e.g., pixels representing the opening in which drops are formed).

Act 810 determines a first length of the drop by counting the number of rows containing pixels corresponding to the second set of pixels within the third image. That is, the drop length is determined to be equal to the last “lit” row in the set of pixels found in Act 809. The first length corresponds to a first estimated drop size.

Act 811 updates a background image using the first image. A low-pass filter may be used to update each pixel's value in the background image. An infinite impulse response filter may be used to update the background image using the first image. A pixel is only updated in the background image for rows below the first length plus a predetermined safety zone. A pixel in the background image is updated by low pass filtering the value from the corresponding pixel in the first image.

Act 812 creates a second thresholded image (e.g., image 772 of FIG. 81) by comparing the first image with the background image. That is, the first image has the background image subtracted from it, and on a pixel-by-pixel basis, the absolute value of each pixel is set to 1 if it is above a second threshold value and is set to a 0 if it is below the second threshold value to generate the second thresholded image.

Act 813 sums the rows of the second thresholded image to create a plurality of row sums (see image 773 of FIG. 81). Each row sum corresponds to a row of the second thresholded image.

Act 814 starts at a row position of the second thresholded image having a first sum of the plurality of sums that corresponds to the first length. The row position is incremented in act 815. Act 816 determines whether the present row position correspond to a corresponding row sum that is below a threshold, e.g., zero. If no, then act 815 is preformed again until the present row position corresponds to a corresponding row sum that is zero and then the method 803 proceeds to act 817.

Act 817 determines a second length is equal to the present row position. The second length corresponding to a second estimated drop size. Act 818 averages the first and second lengths to determine a average length. The average length corresponding to a third estimated drop size. By using the first and second lengths to determine an average length, the effects of condensation on the inner walls of the drip chamber are mitigated. That is, the purpose of creating two estimates of drop length is to compensate for how each length is affected by the presence of condensation. The first length tends to underestimate drop length if a drop of condensation intersects the growing drop from the spigot. The second length tends to overestimates the drop length if the drop of condensation intersects the growing drop from the spigot. Their average provides a better estimate when condensation is present. In the absence of condensation, the estimates are almost equal. In other embodiments, only either the first or second length is used to estimate the drop size.

FIG. 83 shows a flow chart diagram of a method 900 for reducing noise from condensation in accordance with an embodiment of the present disclosure. Method 900 includes acts 902-910.

Act 902 captures an image of a drip chamber. Act 904 performs a canny, edge-detection operation on the image to generate a first processed image. Act 906 performs an AND-operation on a pixel on a first side of an axis of the first processed image with a corresponding mirror pixel on the second side of the axis of the first processed image. That is, Act 902 defines an axis in the first process image, and performs an AND on each pixel on one side with a pixel on the other side, such that the pixel on the other side is symmetrical with the pixel on first side. For example, a 40 (X-axis) by 40 (Y-axis) image may have an axis defined between pixel columns 19 and 20. The top, left pixel would be pixel (1, 1) A pixel at location (1, 5) would be AND-ed with a pixel at (40,5). The resulting pixel would be used for both locations (1, 5) and (40,5) to generate the second processed image.

After act 906 is performed, act 908 determines whether all of the pixels have been processed. Act 908 repeats act 906 until all pixels have been processed. Act 910 provides a second processed image that is the results of all of the AND operations.

FIGS. 84-111 collectively illustrate the physical configuration, integration, and operating principles of the infusion system 2000, its controller 2002, and the compatible administration set 2004. FIG. 84 provides a perspective view of the complete assembly, showing the viewing window, optical monitoring chamber, and primary reference components mounted on the supporting pole 2006. FIGS. 85A-85E present orthogonal external views of the controller 2002, identifying the clinician-facing display, keypad, status indicator, and rear door that provides access to the drip-chamber cavity. FIG. 86 schematically depicts the administration set 2004, which may include a vented spike, filter, transparent drip chamber 2004C with spout, multi-lumen flow-control insert 2034 (FCI), roller clamp 2038, slide clamp, tubing, and distal luer—and indicates how these components couple to the controller for gravity-driven delivery.

FIG. 87 shows the assembled integration of the set 2004 with the controller 2002, illustrating the seated chamber, optical monitoring cavity, and alignment of the FCI between the flow-control (FCV) and safety-occluder (SOV) pinch heads. FIGS. 88 and 95 detail the slide-clamp insertion sequence, demonstrating how the line is first occluded before the door latch is released to ensure safe loading and unloading. FIG. 89 illustrates the loading of the primed chamber and the closure of the door that establishes a defined optical pose and guides the tubing through the valve region. FIGS. 90 and 91 display representative user-interface U1 views showing the programmed rate, volume-to-be-infused (VTBI), time remaining, status icons, and the backlit chamber cavity. FIG. 92 depicts coupling of the chamber and routing of the FCI between the opposing pinch heads, while FIG. 93 shows the priming operation to the prime-level ring that ensures optical clarity and stable meniscus tracking. FIG. 94 provides successive cross-sections of the multi-lumen insert under compression, illustrating its substantially linear area-displacement response and cooperation with an optional anti-pinch member that prevents localized collapse and enhances linearity.

FIG. 96 is an exploded perspective of the administration set 2004, identifying material interfaces and packaging elements suitable for sterilization. FIG. 96 illustrates an exemplary embodiment of an intravenous (IV) administration set configured for gravity-based fluid delivery. The set comprises a proximal end featuring a universal spike (1), which is adapted to sterilely pierce a fluid container. The spike (1) is associated with an air vent cap (3) that can be opened to allow atmospheric pressure to enter rigid containers, and the spike itself is protected by a removable protective cap (2). The spike (1) is fluidly coupled to a transparent drip chamber (4), which is configured to accumulate a reservoir of fluid and allow for visual monitoring of the fluid's drop rate, with said drip chamber (4) further including priming range markers (5) to indicate a proper fluid level. A length of flexible tubing (6) extends from the distal end of the drip chamber (4) to provide the primary fluid path. Disposed along the length of the tubing (6) is a slide clamp (shown immediately distal to the drip chamber) for binary, on-off flow occlusion, and a roller clamp (7) which is adjustably configured to precisely regulate the fluid flow rate through the tubing (6). The distal end of the flexible tubing (6) terminates in a male Luer lock adapter (8), which is configured for secure, leak-proof connection to a patient's vascular access port and is covered by a second protective cap (2) to maintain sterility prior to use.

FIG. 97 diagrams the dual-processor controller architecture CA, which may comprise a vision core, real-time control core, watchdog circuit, and backup-power domain. FIGS. 98 through 100 show the fiducial and prime-level optical checks that verify chamber loading and detect the refraction-based width change associated with correct fill height. FIG. 101 outlines the active-infusion regions of interest (ROIs) used by the vision system for spout, meniscus, and lower-impact tracking. FIGS. 102 through 106 illustrate the image-processing pipeline from raw-frame acquisition through edge extraction, boundary fitting, and stream detection. Finally, FIGS. 107 through 111 present the control-side computations that map observed drop-level and black-pixel data to estimated drop volume, demonstrate regression weighting and residual gating, and show zero-cross timing used to generate the composite real-time flow estimate. Where similar valve, clamp, and optical principles appear in the present application and those fully incorporated by reference, those teachings are expressly incorporated by reference to provide full enablement of the fluid-control mechanisms and drop-analysis methods disclosed herein.

An infusion system 2000 includes a reusable controller 2002 and a compatible disposable administration set 2004 that together implement gravity-driven delivery with closed-loop regulation. The controller 2002 presents a clinician-facing interface with a display 2054, keypad 2056, and status indicator 2058, and provides a front viewing window 2002w into an optical monitoring chamber 2008. A rear door 2018 opens to a drip-chamber cavity that receives a transparent drip chamber in a repeatable pose relative to an optical path of at least one image sensor 2016. For bedside mounting, the controller can be suspended from an IV pole 2006 and additionally secured by an attachment strap, cable or tether 2007 that engages a keyed recess or strap holder 2007a formed atop the controller housing 2002a, providing a redundant retention path during loading, transport, or patient movement.

Shaped registration features 2126—for example, trident-shaped tines 2022—set chamber height and angle so that the spout and lower reservoir fall within predetermined regions of interest (ROIs): a spout ROI 2024, a meniscus ROI 2026, and a lower impact ROI 2028 where drops puddle in a portion of the drip chamber which may be referred to as a puddle. Closing the door locks the chamber in that pose and routes downstream tubing so that a multi-lumen flow-control insert (FCI) 2034 passes between a motorized flow-control valve (FCV) 2030 and an independent safety occluder valve (SOV) 2032. Each valve can fully occlude the line, e.g., at the flow control insert 2034 (which as discussed herein may include multiple lumens to facilitate a linearized flow restriction of the line and as is representatively illustrated best from left to right in FIG. 94 as it is compressed at a pinch location), as each valve is transitioned toward a backstop 2042 which may include stationary pinch heads at positions opposing those of each respective valve 2030, 2032; on a high-priority condition both close, and a watchdog 2048 powered by a backup source 2052 autonomously commands SOV closure if the heartbeat from a real-time control processor 2046 is missed or a main power rail 2050 drops. These architectural and safety domains correspond to the controller organization described in the device record and figures herein.

FIGS. 86-87, 94, 96 depict the administration set 2004 and pinch-site mechanics. The set 2004 may include a vented spike and filter 2005 feeding the transparent drip chamber 2004C with an upper spout 2009, which may extend at least partially into the transparent drip chamber 2004C, a silicone multi-lumen FCI 2034 at a pinch location, a roller clamp 2038, a slide clamp 2036, standard tubing, and a distal luer 2040. A prime-level ring 2066 on the chamber sets a fill height high enough for optical access yet low enough to mitigate entrained air. The line routes so that the FCI lies between the FCV 2030 and SOV 2032 when the door 2018 is closed. The slide clamp 2036 interfaces with the door: inserting the clamp into a slot 2036a first occludes the tubing and then releases the latch, ensuring the line is closed before access (FIGS. 88, 95). The FCV compresses the multi-lumen insert against a backstop so that it can be pinched, preferably in a laminar manner as facilitated by that portion of the tube having multiple lumens; successive cross-sections (FIG. 94) show a substantially linear area-displacement relation compared with single-lumen tubing, enabling simpler, more stable regulation.

Controller architecture and illumination (FIG. 97; FIGS. 85A-85E; FIGS. 98-101). A dual-domain architecture 2108 separates vision from real-time control. A vision core 2044 drives an infrared emitter 2012 behind a contrasting wall 2010 with diffuser 2014, controls the image sensor 2016, and logs to non-volatile memory 2080. A real-time core 2046 actuates FCV 2030 and SOV 2032, executes the regulator, and provides a heartbeat to a watchdog 2048 sustained by backup power 2052. The wall 2010 presents fiducials (e.g., trident 2124 which helps to properly seat the drip chamber by using the spout visible therein as a reference point which obstructs view of the trident when the drip chamber is properly seated99
that define a silhouette frame 2021; localized dimming blanks fiducials within the spout ROI 2024 during estimation frames so the background is substantially uniform there, while features outside remain visible for trident and pose checks. For example, the optical sensor or camera 2016 may make a determination that the drip chamber 2004C is properly disposed or seated within the monitoring chamber of the controller 2002 when the spout 2009 of the administration set 2004 blocks the trident or other fiducial markers from its view; the spout 2009 may also facilitate providing a baseline for tracking drop growth as the system may utilize the known static or stationary position of the spout 2009 of the properly seated drip chamber 2004C for any baseline or reference point for tracking and/or measurement.

The camera 2016 may include an IR band-pass filter and may be obliquely mounted 5-45°, for example, behind a transparent wall of the monitoring chamber, to reduce neck occlusion while maintaining meniscus visibility. Pre-infusion routines (FIGS. 98-100) include an exposure sweep, chamber-pose-spout-geometry verification, and prime-level detection via a refraction-induced width change at a known backlit bar 2122; a chamber state is assigned and infusion is inhibited until valid. Image 99W illustrates a view of the monitoring chamber and the contrasting wall as seen by the image sensor without the drip chamber loaded; Image 991 illustrates a view of the monitoring chamber as seen by the image sensor with the drip chamber properly loaded. In particular, shape registration features 2126 may include a trident 2124 that is obscured by the spout of the drip chamber when properly seated in the controller. FIG. 99 is an image from the Camera used for the Empty Fiducial Check (left), and Loaded Fiducial Check (right), and FIG. 98 shows the contrasting wall as seen by the camera or image sensor with no Drip chamber installed (left), and when a primed Drip Chamber is installed (right). FIG. 100 illustrates the camera image of the Prime Level ROI for Drip Chambers outside of the acceptable prime range (left and right) and properly primed (middle).

Active-infusion vision pipeline (FIGS. 101-106). During infusion (FIG. 101) the processor interleaves frames across the three ROIs. Spout-ROI frames are filtered to suppress glare/condensation, edges are extracted, splash ejecta are masked, and a drop mask is segmented (FIG. 103). Intersection voting localizes a pendant center and leading edge (FIG. 104). A neck-span tracker adaptively crops/scales to hold a target pixel span, bounding sub-pixel edge error, drop-level and black-pixel count signals characterize growth and detachment (FIG. 105). Continuous-stream detection fits substantially parallel lateral edges emerging from the spout, accumulates persistence evidence, and, on threshold, reduces commanded flow and escalates a high-priority alarm (FIG. 106).

As described herein, flow estimation may be determined in real-time and without an explicit volume integration or calculation. For example, om a embodiment, a drop remains attached, the controller fits a sparse perimeter spline to the pendant boundary and computes geometric functionals Ck(t) for example, neck span, truncated silhouette area above a chord, vertical centroid, neck-segment arc length, and curvature at a neck saddle—to form an instantaneous flow proxy:

$${q}_{inst} ( t ) = {a}_{0} + {\sum }_{k} {a}_{k} ( {C}_{k} ( t ) ) \frac{{dC}_{k}}{dt} .$$

A polygonal truncated area above a baseline chord is conveniently computed by the shoelace sum:

$$A = \frac{1}{2} {\sum }_{i} ( {x}_{i} {y}_{i + 1} - {x}_{i + 1} {y}_{i} ) .$$

As shown in FIG. 102, a frame-by-frame optical measurement is employed to capture the evolving morphology of a pendant fluid droplet dispensing from an orifice (e.g., a cannula or nozzle) and growing inside the drip chamber. A high-resolution imaging system, such as a digital camera, acquires a temporal sequence of images, where each image, or frame, captures the instantaneous profile of the forming drop. This creates a digitized, two-dimensional geometric contour corresponding to the fluid-air (or fluid-ambient medium) interface at discrete points in time.

For each of these sequentially acquired frames, the system introduces and utilizes a physics-consistent pendant reference. This reference is not merely a geometric fit but is derived directly from a fundamental principle of fluid mechanics governing static or quasi-static fluid interfaces: the Young-Laplace (Y-L) equation to provide a precise, dynamic method for characterizing a fluid droplet forming inside a drip chamber by integrating visual measurement with fundamental physical laws. Specifically, the system utilizes a sequence of camera images to capture the frame-by-frame geometric contour of the growing pendant droplet. For each of these optically acquired profiles, a physics-consistent reference-based on the Young-Laplace principle—is applied. This principle describes the precise, ideal shape a fluid interface must assume when governed solely by the balance of gravity and surface tension. By algorithmically fitting the experimentally measured droplet contour to this theoretical Young-Laplace shape, the system achieves high-fidelity validation, ensuring that the measurement is not corrupted by optical noise or transient artifacts, but is instead physically sound. This crucial step allows the apparatus to accurately and non-invasively determine key fluid properties, such as surface tension or precise volume, establishing an invariant metrological standard that significantly enhances the accuracy and repeatability of the fluid delivery system.

In particular, a physics track may computes a reference boundary using arc-length parameterization s with radial coordinate r(s), vertical coordinate z(s), and tangent angle φ(s):

$$\frac{dr}{ds} = cos φ , \frac{dz}{ds} = sin φ , \frac{d φ}{ds} = \frac{{p}_{L}}{γ} - \frac{Δ ρ g z}{γ} - \frac{sin φ}{r} ,$$

subject to apex conditions

$$r ( 0 ) = 0 , z ( 0 ) = 0 , φ ( 0 ) = 0.$$

Given measured height h and width w, a solver searches for apex pressure p_{L}* such that the predicted width at z=h matches w; the integrated boundary's residual against the imaged outline provides a physics-based confidence for fusion.

Confidence-weighted fusion and supervision (FIGS. 107-111). Per-track estimates may include spline non-volume, physics fit, background reference, reference-frame subtraction, zero-cross timing, and meniscus trend—are combined as

$$\hat{q} ( t ) = \frac{{\sum }_{m} {w}_{m} ( t ) {q}_{m} ( t )}{{\sum }_{m} {w}_{m} ( t )} .$$

Control-side plots (FIGS. 107-110) illustrate mapping from detected drop-level to per-drop volume, rolling totals for regression, per-point weights, and the weighted fit that yields a real-time rate estimate; FIG. 111 shows zero-cross timing on the drop-level and black-pixel signals as a supervisory channel.

Learning-enabled boundary emission. A neural model can emit spline control points using a composite loss

$$L = α {L}_{shape} + β {L}_{order} + γ {L}_{repulaion} .$$

Control-side plots (FIGS. 107-110) illustrate mapping from detected drop-level to per-drop volume, rolling totals for regression, per-point weights, and the weighted fit that yields a real-time rate estimate; FIG. 111 shows zero-cross timing on the drop-level and black-pixel signals as a supervisory channel.

Learning-enabled boundary emission. A neural model can emit spline control points using a composite loss

$$L = α {L}_{shape} + β {L}_{order} + γ {L}_{repulaion} ,$$

in which Lshape uses uses symmetric Chamfer distance, Lorder preserves traversal order and edge direction, and Lrepulsion penalizes control-point crowding; neck control points can be given higher weights. This preserves geometric fidelity and structural coherence while preventing degenerate configurations in attached-drop intervals.

The complete fluidic measurement system achieves unparalleled accuracy by seamlessly integrating advanced learning-enabled boundary emission with a physics-consistent pendant reference. Initially, the droplet's contour is captured in a frame-by-frame optical measurement within the drip chamber. A specialized neural model then processes this image data, directly outputting the droplet's edge as a smooth, continuous curve defined by spline control points. This model is robustly trained using a composite loss function-incorporating Lshape for geometric fidelity (via symmetric Chamfer distance), Lorder for structural coherence, and Lrepulsion to prevent mathematically unstable, degenerate configurations from control-point crowding—thereby ensuring the digitized boundary is highly precise, even during complex attached-drop intervals. Following this precise geometric definition, the resulting contour is subjected to a validation step using the Young-Laplace principle. This principle serves as an invariant physical standard, mathematically confirming that the neural model's output conforms to the fundamental balance between surface tension and gravity, which guarantees that the derived parameters, such as volume or surface tension, are not just accurate visual representations, but are also physically sound and metrologically consistent.

Baseline-referenced and connectivity-enabled variant. A geometric baseline (spout tangent or meniscus level) defines a local frame for functionals. A connectivity-enabled estimator derives instantaneous flow from temporal change of a baseline-connected characteristic C(t):

$${q}_{inst} ( t ) = K ( C ) \frac{dC}{dt} + b ( C ) ,$$

with k(C) and b(C) calibrated by fluid-aware parameters (e.g., nominal drop factor, viscosity/surface-tension proxies).

In other words, a baseline-referenced and connectivity-enabled method for dynamically estimating the instantaneous fluid flow rate. The core of this method involves establishing a geometric baseline, e.g., the spout tangent (the angle or point where the fluid departs the dispensing orifice) or the stable meniscus level within the fluid source-which defines a local frame of reference for subsequent measurements. This baseline provides a reliable, constant datum against which the dynamic changes in the droplet are measured, thereby minimizing errors caused by camera movement or long-term drift. The system then employs a connectivity-enabled estimator to derive the instantaneous flow rate from the temporal change of a specific, baseline-connected characteristic, C(t). This characteristic, which tracks features like the growing volume or the length of the attached droplet's neck, is intrinsically linked to the flow from the source, making the estimation highly sensitive to the fluid delivery. The estimated flow rate is determined by a linear function of the characteristic, utilizing calibrated coefficients, k(C) and b(C), which are not arbitrary but are calibrated by fluid-aware parameters. These parameters incorporate known fluid properties, such as the nominal drop factor, viscosity, and surface-tension proxies, ensuring the estimation model is thermodynamically and kinematically consistent with the specific fluid being dispensed.

Advantageously, the baseline-referenced and connectivity-enabled method is its significant improvement in the accuracy and stability of instantaneous flow rate calculation over traditional visual methods. By using a geometric baseline, such as the spout tangent or meniscus level, the system establishes a fixed, reliable local frame of reference. This eliminates measurement drift that could otherwise be introduced by minuscule shifts in the camera or dispensing apparatus over time, ensuring that all subsequent measurements of the droplet are consistently anchored. Furthermore, the use of a connectivity-enabled estimator focuses the flow calculation on a characteristic, $\text{C}(\text{t})$, that is directly linked to the fluid's connection point to the source. This characteristic is highly sensitive to the temporal change in dispensed volume. The estimated flow rate is not derived from a simple, uncorrected visual curve fit, but through calibrated coefficients, $\text(k)(\text(C))$ and $\text{b}(\text(C))$, which are tuned using fluid-aware parameters like nominal drop factor, viscosity, and surface tension proxies. This calibration step injects the known physical properties of the fluid into the estimation model, ensuring the calculation is not merely a geometric observation but a physically consistent, robust prediction of the actual fluid dynamics, thereby providing a highly reliable measure of fluid delivery.

Multi-source orchestration. When multiple controllers feed a junction upstream of the patient line, an orchestration processor maintains a commanded patient-level rate by applying virtual head-height equalization and executing verified-closure handoffs. Hydrostatic differences are compensated by

$${H}_{virtual} = {H}_{physical} + \frac{Δ P}{ρ g} .$$

Ripple and stream signatures in non-active lines may veto a handoff; authenticated updates are logged with timestamps and operator credentials.

The Multi-Source Orchestration process may facilitate precisely controlling and transitioning the delivery of fluids when multiple independent controllers (such as separate infusion pumps) are connected upstream of a common junction or manifold leading to the patient line. A central orchestration processor manages this complex arrangement, operating with the primary goal of maintaining a specific, commanded patient-level flow rate regardless of the source being used. A critical function of this processor is applying virtual head-height equalization, a technique used to compensate for hydrostatic differences that naturally arise when fluid sources are positioned at different vertical heights. By dynamically adjusting the control parameters of each pump based on its source's effective head pressure, the system ensures that the fluid delivery pressure remains consistent across all connected lines. Furthermore, the system manages seamless transitions between sources by executing verified-closure handoffs. During a handoff, the processor ensures that the flow from the current, deactivating line is completely and successfully shut off before the new line is fully activated. To ensure patient safety, the system constantly monitors ripple and stream signatures—subtle pressure fluctuations or flow characteristics—in the non-active lines. If any signature suggests fluid leakage or an incomplete closure in a non-active line, the system will veto the handoff, preventing the accidental mixing of fluids or inaccurate dosing. For security and accountability, all authenticated updates and operational changes are meticulously logged with timestamps and operator credentials.

Tube linearity, anti-pinch, and restoration (FIGS. 73A-73C, 74-80, 94). To linearize restriction versus force and prevent localized pinch-off, the downstream tube can include an anti-pinch member [2114]—e.g., a sleeve/shell/bonded insert of higher hoop stiffness with tapered ends—at the pinch site. The FCI [2034] may be disposed adjacent the anti-pinch member so that actuator displacement maps to a substantially linear effective area change over the operating range. To restore a round cross-section after compression, mechanisms include opposed flexible strips with a wrapped string or membrane, a fluid-based bladder with elastomeric fillers, or a geared re-rounding assembly; restoration can be verified by meniscus-drift at zero command or by a calibrated low-rate displacement-versus-flow test. These set mechanics may integrate with the linear area-displacement characteristic portrayed in FIG. 94.

The complete fluidic management system integrates advanced measurement and control into a cohesive architecture for high-precision delivery. The front-end employs a learning-enabled boundary emission technique where a neural model defines the droplet's contour using spline control points optimized by a composite loss function (Lshape, Lorder, Lrepulsion) to achieve robust geometric fidelity even during challenging attached-drop intervals. This boundary is then subjected to a physics-consistent reference check using the Young-Laplace principle, ensuring the measured volume and fluid parameters are not only visually accurate but also physically sound and metrologically consistent. All this precise flow data feeds into the Multi-Source Orchestration processor, which manages concurrent fluid paths from multiple independent controllers feeding a single junction. The processor maintains a stable commanded patient-level flow rate by dynamically applying virtual head-height equalization to compensate for any hydrostatic differences between sources. Furthermore, the system performs verified-closure handoffs, where the transfer of control is conditional, and can be vetoed if ripple and stream signatures indicate leakage in a non-active line, with all authenticated updates securely logged with timestamps and credentials, ensuring both high accuracy and patient safety.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in the drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g., “a,” “an,” or “the,” this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B. This expression signifies that, with respect to the present disclosure, the only relevant components of the device are A and B.

Furthermore, the terms “first,” “second,” “third,” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

1.-110. (canceled)

111. A system for monitoring fluid flow, comprising:
a drip chamber of an administration set configured to dispense a fluid as drops along a generally vertical axis;
a fluid-source interface including an outlet spout positioned to dispense the fluid into an interior of the drip chamber;
a monitoring housing that locates the drip chamber at a defined pose relative to an optical path, the housing including a substantially uniform backlight positioned opposite a camera window;
an image sensor arranged to capture images of drops forming within the drip chamber through the camera window as silhouettes against the substantially uniform backlight; and a processing unit operatively coupled to the image sensor and configured to determine an estimated flow rate during drop formation from time-varying image features of a forming drop without generating an image of the drop using structured pattern data projected into a drop-formation region.

112. The system of claim 111, wherein the substantially uniform backlight lacks spatially structured fiducial markings across a drop-formation region used for flow-rate or volume estimation, and any fiducials present are located outside the drop-formation region and are not used to generate images of the drop using pattern data.

113. The system of claim 111, wherein the processing unit is further configured to:
detect completion of formation of a drop;
derive a geometric representation of the completed drop;
compute a volume of the completed drop from the geometric representation; and validate the estimated flow rate based at least in part on the computed drop volume and a time of drop detachment.

114. The system of claim 113, wherein detecting completion of formation of the drop comprises detecting at least one of:
(i) a neck-thinning rate exceeding a threshold;
(ii) a temporal plateau in pendant-drop growth; and
(iii) a separation event.

115. The system of claim 113, wherein the processing unit derives the geometric representation by segmenting the completed drop into axial slices along the generally vertical axis, determining a cross-sectional area for each axial slice, and summing partial volumes obtained by multiplying the cross-sectional areas by respective slice heights.

116. The system of claim 113, wherein the processing unit derives the geometric representation by fitting a parametric spline boundary to a silhouette of the completed drop and computing drop volume as a solid of revolution of the spline boundary about the generally vertical axis.

117. The system of claim 111, wherein the processing unit is further configured to determine drop volume from empirical data by selecting or interpolating a value from a library indexed by one or more of: drop morphology descriptors, fluid identity, temperature, or optical configuration, the library being calibrated against gravimetric references.

118. The system of claim 111, further comprising a spout-locator module configured to:
initialize a spout position from a first reliably segmented drop;
maintain the spout position over successive frames using a recursive filter; and
relocalize the spout position responsive to deviation from a fiducial-referenced coordinate frame exceeding a threshold.

119. The system of claim 111, further comprising a second image sensor positioned to view the drops at a viewing angle different from that of the image sensor, wherein the processing unit is configured to compute respective per-view volume or flow-rate estimates and to combine the estimates in proportions reflecting per-view confidence measures.

120. The system of claim 119, wherein at least one of the image sensors is mounted at an oblique angle relative to a plane normal to the generally vertical axis to reduce occlusion of a neck region of the drop.

121. The system of claim 111, wherein the processing unit is configured to calibrate camera intrinsics and extrinsics and to compensate volume or flow-rate estimation for camera pose relative to the drip chamber.

122. The system of claim 111, wherein the processing unit is configured to enter a low-flow mode when the estimated flow rate is below a threshold, the low-flow mode comprising increasing temporal averaging, applying integer frame skipping to maintain effective sampling of drop formation without bias, and applying hang-on compensation during extended pendant residence.

123. The system of claim 112, wherein the processing unit, in the low-flow mode, estimates instantaneous volume of a pendant drop during formation from silhouette-derived geometric features without waiting for detachment of the drop.

124. The system of claim 111, further comprising a flow-control insert positioned downstream of the drip chamber, the flow-control insert comprising a multilumen tube including a plurality of conduits extending along its length, each conduit being compressible such that overall flow restriction varies in a comparatively linearized manner under compression relative to a single-lumen tube.

125. The system of claim 114, further comprising an actuator configured to compress the flow-control insert, wherein the processing unit is configured to control the actuator to maintain a target flow rate based at least in part on the estimated flow rate determined from the silhouettes.

126. The system of claim 115, further comprising an independent safety occluder configured to close a flow path upon detection of a fault condition including at least one of: upstream air, downstream occlusion, excessive flow, or power loss, wherein the safety occluder is configured to fail-safe to a closed state upon loss of power.

127. The system of claim 115, wherein the processing unit is configured to regulate flow based on volumetric or geometric estimation from the silhouettes while suppressing drop-count-only signals that would otherwise override volumetric estimation.

128. The system of claim 115, further comprising a medication library storing infusion constraints and fluid parameters, wherein the processing unit is configured to obtain, based on a fluid identity, one or more fluid parameters and one or more infusion constraints from the medication library and to regulate the actuator according to the estimated flow rate subject to the infusion constraints.

129. The system of claim 111, further comprising:
an audit-logging module configured to, for each flow-rate or volume estimate used for therapy, record at least a model-version identifier, calibration-version identifiers, camera parameters, a timestamp, and one or more frame-integrity indicators, and to exclude image frames from estimation responsive to detecting at least one of: repeated frames, missing timestamps, or structural similarity above a threshold over a temporal window.

130. A method for monitoring fluid flow through a drip chamber, comprising:
locating a drip chamber of an administration set in a monitoring housing that provides a substantially uniform backlight opposite a camera window;
capturing, with an image sensor viewing the drip chamber through the camera window, images of drops forming within the drip chamber as silhouettes against the substantially uniform backlight;
extracting time-varying geometric features of a forming drop from the images while the drop remains attached to an outlet spout; and
determining an estimated flow rate during drop formation from the time-varying geometric features without generating an image of the drop using structured pattern data projected into a drop-formation region.

131. The method of claim 130, further comprising:
detecting completion of formation of a drop;
constructing a geometric representation of the completed drop;
computing a volume of the completed drop from the geometric representation; and
reconciling the estimated flow rate during formation with a flow rate derived from the computed drop volume and a detachment time.
